SEQUENCE LISTING (37 sequences)

SEQ ID NO: 1 - DNA, 1680 bp, synthetic construct
  source 1..1680: mol_type = other DNA
gacactctgtgcatcggataccacgctaacaactccaccgacactgtggacaccgtcctggagaagaacgtcaccgtgacccactctgtgaacctgctggaggacaagcacaacggcaagctgtgcaagctgagaggcgtggccccactgcacctgggcaagtgcaacatcgctggctggatactgggtaaccctgagtgcgaaagcctgtccactgctagatcatggtcctacatcgtggaaacttccaacagcgacaacggtacttgctacccaggagacttcatcaactacgaggagctgagggagcagctgtcatctgtgtccagcttcgaaaggttcgaaatcttcccaaagacttcatcttggcctaaccacgacagcgacaagggtgtgactgctgcctgccctcacgctggtgccaagtcattctacaagaacctgatctggctggtgaagaagggcaactcataccctaagctgaaccagacttacatcaacgacaagggcaaggaggtcctggtgctgtggggtatccaccaccctcccaccatcgctgcccaggagtctctgtaccagaacgctgacgcttacgtgttcgtcggtacttcacgctactccaagaagttcaagcccgaaatcgctactcgcccaaaggtgcgcgaccaggaaggcaggatgaactactactggactctggtggaacctggagacaagatcaccttcgaggctactggaaacctggtcgtgccaagatacgctttcactatggagcgtgacgctggttctggcatcatcatctccgacactcctgtccacgactgcaacaccacttgccagaccccagagggtgctatcaacacctctctgccattccagaacgtgcacccaatcaccatcggaaagtgccccaagtacgtgaagtctaccaagctgcgtctggctactggactgaggaacgtgccatctatccagtcacgcggtctgttcggtgctatcgctggcttcatcgagggcggttggactggcatggtggacggttggtacggataccaccaccagaacgagcagggctcaggttacgctgctgacctgaagtcaacccagaacgctatcgacaagatcactaacaaggtgaactctgtgatcgaaaagatgaacacccagttcaccgctgtgggaaaggagttcaaccacctggagaagcgtatcgagaacctgaacaagaaggtggacgacggattcctggacatctggacctacaacgctgagctgctggtcctgctggaaaacgagcgcaccctggactaccacgacagcaacgtcaagaacctgtacgagaaggtgcgcaaccagctgaagaacaacgctaaggagatcggcaacggctgcttcgagttctaccacaagtgcgacaacacttgcatggaatccgtgaagaacggtacttacgactacccaaagtactctgaggaggccaagctgaacagggagaagatcgacggcgtgaagctggagtccactcgcatctaccagatcctggctatctactcaactgtggcctcttcactggtgctggtcgtgtctctgggtgctatcagcttctggatgggttcagctggtctcgaagttttgttccagggaccccatcaccaccatcatcaccaccaccatcactaa

SEQ ID NO: 2 - protein, 559 aa, synthetic construct
DTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMGSAGLEVLFQGPHHHHHHHHHH

SEQ ID NO: 3 - DNA, 1677 bp, synthetic construct
  source 1..1677: mol_type = other DNA
cagaagatccccggcaacgacaactcaaccgctactctgtgcctgggacaccacgccgtgccaaacggcaccatcgtcaagaccatcactaacgaccgtatcgaggtgaccaacgctactgaactggtccagaactccagcatcggagaaatctgcgactccccacaccagatcctggacggtggcaactgcactctgatcgacgctctgctgggcgaccctcagtgcgacggattccagaacaagaagtgggacctgttcgtggaacgctctcgtgcctactcaaactgctacccttacgacgtgcccgactacgcttctctgaggtcactggtcgcctcttcaggaaccctggagttcaagaacgaatctttcaactgggctggtgtcactcagaacggcaagtccttcagctgcatccgtggttccagctcttcattcttctcaaggctgaactggctgacccacctgaactacacttaccccgccctgaacgtgaccatgccaaacaaggagcagttcgacaagctgtacatctggggagtccaccaccccggtactgacaaggaccagatcagcctgtacgctcagtccagcggtagaatcaccgtgtccactaagcgcagccagcaggccgtcatccccaacatcggcagcaggccaagaatccgcgacatcccttccagaatcagcatctactggaccatcgtgaagccaggcgacatcctgctgatcaactccactggcaacctgatcgctcctcgtggatacttcaagatcaggtccggcaagtcctccatcatgcgcagcgacgcccctatcggaaagtgcaagtctgagtgcatcacccccaacggttcaatcccaaacgacaagcctttccagaacgtgaaccgtatcacttacggcgcttgccctcgttacgtcaagcagaacaccctgaagctggccactggaatgagaaacgtgcccgaaaagcagacccgcggtatcttcggcgctatcgccggcttcatcgagaacggatgggagggtatggtggacggctggtacggattccgtcaccagaactccgagggaaggggtcaggctgccgacctgaagagcacccaggctgccatcgaccagatcaacggcaagctgaacagactgatcggaaagactaacgaaaagttccaccagatcgagaaggagttcagcgaggtggaaggtcgcgtccaggacctggagaagtacgtggaggacaccaagatcgacctgtggtcctacaacgctgagctgctggtcgccctggaaaaccagcacaccatcgacctgactgacagcgagatgaacaagctgttcgaaaagaccaagaagcagctgcgtgagaacgctgaggacatgggcaacggatgcttcaagatttaccacaagtgcgacaacgcctgcatcggttctatcaggaacgagacttacgaccacaacgtgtacagagacgaagctctgaacaaccgcttccagatcaagggtgtcgagctgaagtcaggctacaaggactggatcttgtggatctctttcgccatctcatgcttcctgctgtgcgtggctctgctgggtttcatcatgggttcagctggtctcgaagttctgttccaaggaccgcaccatcatcaccatcaccatcatcatcactaa

SEQ ID NO: 4 - protein, 558 aa, synthetic construct
QKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMGSAGLEVLFQGPHHHHHHHHHH

SEQ ID NO: 5 - DNA, 1728 bp, synthetic construct
  source 1..1728: mol_type = other DNA
gacaggatctgcaccggtatcacttccagcaacagcccccacgtggtcaagactgctacccagggagaagtgaacgtcactggtgtgatccccctgaccactaccccaaccaagtctcacttcgccaacctgaagggcactgaaacccgcggcaagctgtgcccaaagtgcctgaactgcactgacctggacgtggctctgggcagacccaagtgcaccggaaagatcccatccgcccgcgtcagcatcctgcacgaagtgcgtcctgtcacttctggctgcttccccatcatgcacgaccgcaccaagatccgtcagctgcctaacctgctgaggggttacgagcacgtgagactgtcaactcacaacgtcatcaacgctgaggacgccccaggccgcccttacgagatcggcacctccggcagctgccccaacatcactaacggaaacggtttcttcgctactatggcttgggccgtcccaaagaacaagactgccaccaaccctctgaccatcgaagtgccctacatctgcaccgagggcgaggaccagatcactgtctggggattccactccgacaacgagactcagatggctaagctgtacggcgacagcaagcctcagaagttcacctcttcagccaacggagtgactacccactacgtctcccagatcggtggcttcccaaaccagaccgaggacggaggtctgcctcagtctggtcgtatcgtggtggactacatggtgcagaagtcaggaaagactggcaccatcacttaccagcgcggaatcctgctgcctcagaaagtgtggtgcgcttctggtcgttcaaaggtcatcaagggttccctgcccctgatcggtgaagctgactgcctgcacgagaagtacggcggactgaacaagagcaagccctactacactggagaacacgctaaggccatcggtaactgcccaatctgggtcaagactcctctgaagctggctaacggcaccaagtacaggcctcccgccaagctgctgaaggagagaggcttcttcggagctatcgccggtttcctggaaggtggctgggagggcatgatcgctggttggcacggctacacctcccacggtgctcacggtgtggctgtggctgccgacctgaagtcaactcaggaagccatcaacaagatcaccaagaacctgaactctctgtcagagctggaagtgaagaacctgcaacgcctgtccggagctatggacgagctgcacaacgagatcctggaactggacgagaaggtggacgacctgcgtgctgacaccatctccagccagatcgaactggccgtcctgctgtccaacgagggaatcatcaacagcgaggacgaacacctgctggctctggaaaggaagctgaagaagatgctgggtccaagcgccgtggaaatcggcaacggatgcttcgaaaccaagcacaagtgcaaccagacttgcctggacagaatcgctgccggtactttcgacgctggcgagttctctctgcctaccttcgactcactgaacatcactgctgcctctctgaacgacgacggcctggacaaccacaccatcctgctgtactactcaactgctgcctcttcactggctgtcaccctgatgatcgccatcttcgtggtcggttcagctggtctcgaagttctgttccagggtccgcaccaccatcaccatcaccaccaccatcactaa

SEQ ID NO: 6 - protein, 575 aa, unidentified
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVGSAGLEVLFQGPHHHHHHHHHH

SEQ ID NO: 7 - DNA, 1734 bp, synthetic construct
  source 1..1734: mol_type = other DNA
gacaggatctgcactggtatcacctccagcaactcacctcacgtggtcaagactgctacccagggtgaagtgaacgtcaccggcgtgatccccctgaccactaccccaactaagtcctacttcgccaacctgaagggaactaggacccgcggcaagctgtgccctgactgcctgaactgcaccgacctggacgtggctctgggaaggcccatgtgcgtcggtactaccccatccgctaaggccagcatcctgcacgaggtgcgccctgtcacctctggctgcttccccatcatgcacgacaggactaagatcagacagctgccaaacctgctgcgcggatacgaaaagatccgtctgtcaactcagaacgtcatcgacgctgagaaggccccaggtggcccttaccgcctgggcacttccggaagctgccctaacgctacctccaagatcggcttcttcgccactatggcttgggccgtgcccaaggacaactacaagaacgccactaaccctctgaccgtggaagtcccctacatctgcactgagggagaggaccagatcaccgtctggggtttccacagcgacaacaagacccagatgaagtctctgtacggagactcaaaccctcagaagttcacttcttcagctaacggtgtgactacccactacgtctctcagatcggcgacttcccagaccagaccgaggacggaggtctgcctcagtcaggtcgtatcgtggtggactacatgatgcagaagcctggcaagaccggcaccatcgtgtaccagcgcggagtcctgctgccacagaaagtgtggtgcgcttctggtcgttcaaaggtcatcaagggctccctgccactgatcggagaagccgactgcctgcacgaggaatacggcggactgaacaagagcaagccttactacaccggcaagcacgctaaggccatcggcaactgcccaatctgggtcaagacccctctgaagctggctaacggcactaagtacaggcctcccgccaagctgctgaaggagagaggattcttcggtgctatcgctggcttcctggaaggtggctgggagggaatgatcgctggctggcacggatacacctcccacggtgctcacggcgtggctgtggctgccgacctgaagagcacccaggaagccatcaacaagatcactaagaacctgaactctctgtcagagctggaagtgaagaacctgcaacgcctgagcggagctatggacgagctgcacaacgagatcctggaactggacgagaaggtggacgacctgcgtgctgacaccatctccagccagatcgaactggccgtcctgctgtctaacgagggtatcatcaactcagaggacgaacacctgctggctctggaaaggaagctgaagaagatgctgggtccttccgctgtggacatcggaaacggttgcttcgagactaagcacaagtgcaaccagacctgcctggacagaatcgctgccggcaccttcaacgctggagagttctccctgccaactttcgacagcctgaacatcaccgctgcctccctgaacgacgacggtctggacaaccacactatcctgctgtactacagcaccgctgcctcttcactggctgtgactctgatgctggccatcttcatcgtgggttcagctggtctcgaagttctgttccagggtccccatcaccaccatcatcaccatcaccaccactaa

SEQ ID NO: 8 - protein, 577 aa, synthetic construct
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVGSAGLEVLFQGPHHHHHHHHHH

SEQ ID NO: 9 - DNA, 1626 bp, synthetic construct
  source 1..1626: mol_type = other DNA
gacactctgtgcatcggataccacgctaacaactccaccgacactgtggacaccgtcctggagaagaacgtcaccgtgacccactctgtgaacctgctggaggacaagcacaacggcaagctgtgcaagctgagaggcgtggccccactgcacctgggcaagtgcaacatcgctggctggatactgggtaaccctgagtgcgaaagcctgtccactgctagatcatggtcctacatcgtggaaacttccaacagcgacaacggtacttgctacccaggagacttcatcaactacgaggagctgagggagcagctgtcatctgtgtccagcttcgaaaggttcgaaatcttcccaaagacttcatcttggcctaaccacgacagcgacaagggtgtgactgctgcctgccctcacgctggtgccaagtcattctacaagaacctgatctggctggtgaagaagggcaactcataccctaagctgaaccagacttacatcaacgacaagggcaaggaggtcctggtgctgtggggtatccaccaccctcccaccatcgctgcccaggagtctctgtaccagaacgctgacgcttacgtgttcgtcggtacttcacgctactccaagaagttcaagcccgaaatcgctactcgcccaaaggtgcgcgaccaggaaggcaggatgaactactactggactctggtggaacctggagacaagatcaccttcgaggctactggaaacctggtcgtgccaagatacgctttcactatggagcgtgacgctggttctggcatcatcatctccgacactcctgtccacgactgcaacaccacttgccagaccccagagggtgctatcaacacctctctgccattccagaacgtgcacccaatcaccatcggaaagtgccccaagtacgtgaagtctaccaagctgcgtctggctactggactgaggaacgtgccatctatccagtcacgcggtctgttcggtgctatcgctggcttcatcgagggcggttggactggcatggtggacggttggtacggataccaccaccagaacgagcagggctcaggttacgctgctgacctgaagtcaacccagaacgctatcgacaagatcactaacaaggtgaactctgtgatcgaaaagatgaacacccagttcaccgctgtgggaaaggagttcaaccacctggagaagcgtatcgagaacctgaacaagaaggtggacgacggattcctggacatctggacctacaacgctgagctgctggtcctgctggaaaacgagcgcaccctggactaccacgacagcaacgtcaagaacctgtacgagaaggtgcgcaaccagctgaagaacaacgctaaggagatcggcaacggctgcttcgagttctaccacaagtgcgacaacacttgcatggaatccgtgaagaacggtacttacgactacccaaagtactctgaggaggccaagctgaacagggagaagatcgacggcgtgaagctggagtccactcgcatctaccagatcctggctatctactcaactgtggcctcttcactggtgctggtcgtgtctctgggtgctatcagcttctggatgggttcagctggttaa

SEQ ID NO: 10 - protein, 541 aa, synthetic construct
DTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMGSAG

SEQ ID NO: 11 - DNA, 1623 bp, synthetic construct
  source 1..1623: mol_type = other DNA
cagaagatccccggcaacgacaactcaaccgctactctgtgcctgggacaccacgccgtgccaaacggcaccatcgtcaagaccatcactaacgaccgtatcgaggtgaccaacgctactgaactggtccagaactccagcatcggagaaatctgcgactccccacaccagatcctggacggtggcaactgcactctgatcgacgctctgctgggcgaccctcagtgcgacggattccagaacaagaagtgggacctgttcgtggaacgctctcgtgcctactcaaactgctacccttacgacgtgcccgactacgcttctctgaggtcactggtcgcctcttcaggaaccctggagttcaagaacgaatctttcaactgggctggtgtcactcagaacggcaagtccttcagctgcatccgtggttccagctcttcattcttctcaaggctgaactggctgacccacctgaactacacttaccccgccctgaacgtgaccatgccaaacaaggagcagttcgacaagctgtacatctggggagtccaccaccccggtactgacaaggaccagatcagcctgtacgctcagtccagcggtagaatcaccgtgtccactaagcgcagccagcaggccgtcatccccaacatcggcagcaggccaagaatccgcgacatcccttccagaatcagcatctactggaccatcgtgaagccaggcgacatcctgctgatcaactccactggcaacctgatcgctcctcgtggatacttcaagatcaggtccggcaagtcctccatcatgcgcagcgacgcccctatcggaaagtgcaagtctgagtgcatcacccccaacggttcaatcccaaacgacaagcctttccagaacgtgaaccgtatcacttacggcgcttgccctcgttacgtcaagcagaacaccctgaagctggccactggaatgagaaacgtgcccgaaaagcagacccgcggtatcttcggcgctatcgccggcttcatcgagaacggatgggagggtatggtggacggctggtacggattccgtcaccagaactccgagggaaggggtcaggctgccgacctgaagagcacccaggctgccatcgaccagatcaacggcaagctgaacagactgatcggaaagactaacgaaaagttccaccagatcgagaaggagttcagcgaggtggaaggtcgcgtccaggacctggagaagtacgtggaggacaccaagatcgacctgtggtcctacaacgctgagctgctggtcgccctggaaaaccagcacaccatcgacctgactgacagcgagatgaacaagctgttcgaaaagaccaagaagcagctgcgtgagaacgctgaggacatgggcaacggatgcttcaagatttaccacaagtgcgacaacgcctgcatcggttctatcaggaacgagacttacgaccacaacgtgtacagagacgaagctctgaacaaccgcttccagatcaagggtgtcgagctgaagtcaggctacaaggactggatcttgtggatctctttcgccatctcatgcttcctgctgtgcgtggctctgctgggtttcatcatgggttcagctggttaa

SEQ ID NO: 12 - protein, 540 aa, synthetic construct
QKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMGSAG

SEQ ID NO: 13 - DNA, 1674 bp, synthetic construct
  source 1..1674: mol_type = other DNA
gacaggatctgcaccggtatcacttccagcaacagcccccacgtggtcaagactgctacccagggagaagtgaacgtcactggtgtgatccccctgaccactaccccaaccaagtctcacttcgccaacctgaagggcactgaaacccgcggcaagctgtgcccaaagtgcctgaactgcactgacctggacgtggctctgggcagacccaagtgcaccggaaagatcccatccgcccgcgtcagcatcctgcacgaagtgcgtcctgtcacttctggctgcttccccatcatgcacgaccgcaccaagatccgtcagctgcctaacctgctgaggggttacgagcacgtgagactgtcaactcacaacgtcatcaacgctgaggacgccccaggccgcccttacgagatcggcacctccggcagctgccccaacatcactaacggaaacggtttcttcgctactatggcttgggccgtcccaaagaacaagactgccaccaaccctctgaccatcgaagtgccctacatctgcaccgagggcgaggaccagatcactgtctggggattccactccgacaacgagactcagatggctaagctgtacggcgacagcaagcctcagaagttcacctcttcagccaacggagtgactacccactacgtctcccagatcggtggcttcccaaaccagaccgaggacggaggtctgcctcagtctggtcgtatcgtggtggactacatggtgcagaagtcaggaaagactggcaccatcacttaccagcgcggaatcctgctgcctcagaaagtgtggtgcgcttctggtcgttcaaaggtcatcaagggttccctgcccctgatcggtgaagctgactgcctgcacgagaagtacggcggactgaacaagagcaagccctactacactggagaacacgctaaggccatcggtaactgcccaatctgggtcaagactcctctgaagctggctaacggcaccaagtacaggcctcccgccaagctgctgaaggagagaggcttcttcggagctatcgccggtttcctggaaggtggctgggagggcatgatcgctggttggcacggctacacctcccacggtgctcacggtgtggctgtggctgccgacctgaagtcaactcaggaagccatcaacaagatcaccaagaacctgaactctctgtcagagctggaagtgaagaacctgcaacgcctgtccggagctatggacgagctgcacaacgagatcctggaactggacgagaaggtggacgacctgcgtgctgacaccatctccagccagatcgaactggccgtcctgctgtccaacgagggaatcatcaacagcgaggacgaacacctgctggctctggaaaggaagctgaagaagatgctgggtccaagcgccgtggaaatcggcaacggatgcttcgaaaccaagcacaagtgcaaccagacttgcctggacagaatcgctgccggtactttcgacgctggcgagttctctctgcctaccttcgactcactgaacatcactgctgcctctctgaacgacgacggcctggacaaccacaccatcctgctgtactactcaactgctgcctcttcactggctgtcaccctgatgatcgccatcttcgtggtcggttcagctggttaa

SEQ ID NO: 14 - protein, 557 aa, synthetic construct
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVGSAG

SEQ ID NO: 15 - DNA, 1680 bp, synthetic construct
  source 1..1680: mol_type = other DNA
gacaggatctgcactggtatcacctccagcaactcacctcacgtggtcaagactgctacccagggtgaagtgaacgtcaccggcgtgatccccctgaccactaccccaactaagtcctacttcgccaacctgaagggaactaggacccgcggcaagctgtgccctgactgcctgaactgcaccgacctggacgtggctctgggaaggcccatgtgcgtcggtactaccccatccgctaaggccagcatcctgcacgaggtgcgccctgtcacctctggctgcttccccatcatgcacgacaggactaagatcagacagctgccaaacctgctgcgcggatacgaaaagatccgtctgtcaactcagaacgtcatcgacgctgagaaggccccaggtggcccttaccgcctgggcacttccggaagctgccctaacgctacctccaagatcggcttcttcgccactatggcttgggccgtgcccaaggacaactacaagaacgccactaaccctctgaccgtggaagtcccctacatctgcactgagggagaggaccagatcaccgtctggggtttccacagcgacaacaagacccagatgaagtctctgtacggagactcaaaccctcagaagttcacttcttcagctaacggtgtgactacccactacgtctctcagatcggcgacttcccagaccagaccgaggacggaggtctgcctcagtcaggtcgtatcgtggtggactacatgatgcagaagcctggcaagaccggcaccatcgtgtaccagcgcggagtcctgctgccacagaaagtgtggtgcgcttctggtcgttcaaaggtcatcaagggctccctgccactgatcggagaagccgactgcctgcacgaggaatacggcggactgaacaagagcaagccttactacaccggcaagcacgctaaggccatcggcaactgcccaatctgggtcaagacccctctgaagctggctaacggcactaagtacaggcctcccgccaagctgctgaaggagagaggattcttcggtgctatcgctggcttcctggaaggtggctgggagggaatgatcgctggctggcacggatacacctcccacggtgctcacggcgtggctgtggctgccgacctgaagagcacccaggaagccatcaacaagatcactaagaacctgaactctctgtcagagctggaagtgaagaacctgcaacgcctgagcggagctatggacgagctgcacaacgagatcctggaactggacgagaaggtggacgacctgcgtgctgacaccatctccagccagatcgaactggccgtcctgctgtctaacgagggtatcatcaactcagaggacgaacacctgctggctctggaaaggaagctgaagaagatgctgggtccttccgctgtggacatcggaaacggttgcttcgagactaagcacaagtgcaaccagacctgcctggacagaatcgctgccggcaccttcaacgctggagagttctccctgccaactttcgacagcctgaacatcaccgctgcctccctgaacgacgacggtctggacaaccacactatcctgctgtactacagcaccgctgcctcttcactggctgtgactctgatgctggccatcttcatcgtgggttcagctggttaa

SEQ ID NO: 16 - protein, 559 aa, synthetic construct
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVGSAG

SEQ ID NO: 17 - DNA, 1626 bp, synthetic construct
  source 1..1626: mol_type = other DNA
gacaccctgtgcatcggataccacgccaacaactctaccgacactgtggacactgtcctggagaagaacgtgaccgtcactcactccgtgaacctgctggaagacaagcacaacggcaagctgtgcaagctgaggggagtcgctcctctgcacctgggcaagtgcaacatcgccggttggatcttgggcaaccccgagtgcgaatccctgagcaccgctcgctcctggtcatacatcgtggagacctccaacagcgacaacggaacttgctaccctggtgacttcatcaactacgaggaactgagggaacagctgtccagcgtgtcttcattcgagagattcgaaatcttcccaaagacctccagctggcctaaccacgactctgacaacggtgtgactgctgcttgcccacacgctggagccaagtcattctacaagaacctgatctggctggtcaagaagggcaagtcctacccaaagatcaaccagacctacatcaacgacaagggcaaggaggtgctggtcctgtggggcatccaccaccctcccactatcgctgaccagcagtccctgtaccagaacgctgacgcctacgtgttcgtcggaacctctcgctactcaaagaagttcaagcccgagatcgccactcgcccaaaggtgcgtgaccaggaaggtcgtatgaactactactggaccctggtcgagcccggtgacaagatcaccttcgaagctactggcaacctggtggctccacgctacgccttcactatggagcgtgacgccggttccggtatcatcatcagcgacactccagtccacgactgcaacaccacttgccagacccctgaaggtgctatcaacacttcactgcctttccagaacgtgcaccccatcaccatcggaaagtgcccaaagtacgtcaagtccaccaagctgaggctggccactggtctgagaaacgtgccttccatccagagccgtggactgttcggtgctatcgccggattcatcgagggtggctggactggtatggtggacggctggtacggataccaccaccagaacgaacagggcagcggatacgctgccgacctgaagtctacccagaacgctatcgacaagatcactaacaaggtgaacagcgtcatcgagaagatgaacacccagttcactgccgtgggcaaggagttcaaccacctggagaagcgcatcgaaaacctgaacaagaaggtggacgacggattcctggacatctggacctacaacgctgagctgctggtcctgctggagaacgaaaggactctggactaccacgactctaacgtgaagaacctgtacgaaaaggtcagaaaccagctgaagaacaacgccaaggagatcggtaacggctgcttcgagttctaccacaagtgcgacaacacctgcatggagagcgtgaagaacggtacttacgactaccccaagtactctgaggaagctaagctgaaccgcgaaaagatcgacggcgtcaagctggactcaacccgtatctaccagatcctggctatctactccactgtggcctcttcactggtcctggtggtctctctgggcgctatctcattctggatgggttccgctagctag

SEQ ID NO: 18 - protein, 541 aa, synthetic construct
DTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMGSAS

SEQ ID NO: 19 - DNA, 1623 bp, synthetic construct
  source 1..1623: mol_type = other DNA
cagaagatccctggtaacgacaacagcaccgctactctgtgcctgggacaccacgccgtgcccaacggcaccatcgtcaagaccatcactaacgaccgtatcgaggtgaccaacgccactgaactggtccagaactccagcatcggcgaaatctgcgactcccctcaccagatcctggacggtggcaactgcactctgatcgacgctctgctgggcgaccctcagtgcgacggattccagaacaaggagtgggacctgttcgtggaacgctctcgtgccaactcaaactgctacccttacgacgtgcccgactacgcttccctgaggagcctggtcgcctcttcaggcaccctggagttcaagaacgaatccttcaactggaccggagtcaagcagaacggtacttccagcgcttgcatccgtggctcttcatccagcttcttcagcaggctgaactggctgacccacctgaactacacttaccccgccctgaacgtgaccatgccaaacaacgagcagttcgacaagctgtacatctggggcgtccaccacccatctactgacaaggaccagatcagcctgttcgctcagcctagcggcagaatcaccgtgtccactaagcgcagccagcaggccgtcatccccaacatcggatctaggccaagaatccgcgacatcccttcccgcatctccatctactggaccatcgtgaagcccggagacatcctgctgatcaactcaactggcaacctgatcgctccacgtggatacttcaagatcaggtctggcaagtcctccatcatgcgctcagacgcccctatcggcaagtgcaagtccgagtgcatcacccccaacggcagcatcccaaacgacaagcctttccagaacgtgaaccgtatcacttacggagcttgcccacgttacgtcaagcagagcaccctgaagctggccactggtatgagaaacgtgcctgaaaagcagacccgcggtatcttcggcgctatcgccggcttcatcgagaacggatgggagggtatggtggacggctggtacggattccgtcaccagaactctgagggaaggggtcaggctgccgacctgaagtcaacccaggctgccatcgaccagatcaacggcaagctgaacagactgatcggaaagactaacgaaaagttccaccagatcgagaaggagttctccgaggtggaaggtcgcgtccaggacctggagaagtacgtggaagacactaagatcgacctgtggtcttacaacgctgagctgctggtcgccctggaaaaccagcacaccatcgacctgactgactcagagatgaacaagctgttcgaaaagaccaagaagcagctgcgtgagaacgctgaagacatgggcaacggatgcttcaagatttaccacaagtgcgacaacgcctgcatcggttccatcaggaacgagacttacgaccacaacgtgtacagagacgaagctctgaacaaccgcttccagatcaagggtgtcgagctgaagagcggctacaaggactggatcttgtggatctctttcgctatgtcatgcttcctgctgtgcatcgccctgctgggtttcatcatgggctctgctagctag

SEQ ID NO: 20 - protein, 540 aa, synthetic construct
QKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYTYPALNVTMPNNEQFDKLYIWGVHHPSTDKDQISLFAQPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMGSAS

SEQ ID NO: 21 - DNA, 1686 bp, synthetic construct
  source 1..1686: mol_type = other DNA
gacaccctgtgcatcggctaccacgctaacaacagcaccgacactgtggacactgtcctggagaagaacgtgaccgtcactcacagcgtgaacctgctggaagacaagcacaacggcaagctgtgcaagctgaggggagtcgctcctctgcacctgggcaagtgcaacatcgccggttggatcttgggcaaccccgagtgcgaatccctgagcaccgctcgctcctggtcatacatcgtggaaacctccaacagcgacaacggaacttgctaccctggtgacttcatcaactacgaggaactgagggaacagctgtccagcgtgtcttcattcgagagattcgaaatcttcccaaagacctccagctggcctaaccacgactctgacaacggtgtgactgctgcttgcccacacgctggagccaagtctttctacaagaacctgatctggctggtcaagaagggcaagtcatacccaaagatcaaccagacctacatcaacgacaagggcaaggaggtgctggtcctgtggggcatccaccaccctcccactatcgctgaccagcagtccctgtaccagaacgctgacgcctacgtgttcgtcggaacctcccgctacagcaagaagttcaagcccgagatcgccactcgcccaaaggtgcgtgaccaggaaggtcgtatgaactactactggaccctggtcgagcccggtgacaagatcaccttcgaagctactggcaacctggtggctccacgctacgccttcactatggagcgtgacgccggttccggtatcatcatcagcgacactccagtccacgactgcaacaccacttgccagacccctgaaggagctatcaacacttctctgcctttccagaacgtgcaccccatcaccatcggaaagtgcccaaagtacgtcaagtcaaccaagctgaggctggccactggtctgagaaacgtgccttctatccagtcacgtggactgttcggtgctatcgccggattcatcgagggtggctggactggtatggtggacggctggtacggataccaccaccagaacgaacagggctccggatacgctgccgacctgaagagcacccagaacgctatcgacaagatcactaacaaggtgaactccgtcatcgagaagatgaacacccagttcactgccgtgggcaaggagttcaaccacctggagaagcgcatcgaaaacctgaacaagaaggtggacgacggattcctggacatctggacctacaacgctgagctgctggtcctgctggagaacgaaaggactctggactaccacgacagcaacgtgaagaacctgtacgaaaaggtcagaaaccagctgaagaacaacgccaaggagatcggtaacggctgcttcgagttctaccacaagtgcgacaacacctgcatggagtccgtgaagaacggtacttacgactaccccaagtacagcgaggaagctaagctgaaccgcgaaaagatcgacggcgtcaagctggactctacccgtatctaccagatcctggctatctactcaactgtggcctcttcactggtcctggtggtgtccctgggcgctatctcattctggatgggatctgctagcctggaggttctcttccagggtccaggatcccatcaccaccatcatcaccatcaccaccactaa

SEQ ID NO: 22 - protein, 561 aa, synthetic construct
DTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMGSASLEVLFQGPGSHHHHHHHHHH

SEQ ID NO: 23 - DNA, 1683 bp, synthetic construct
  source 1..1683: mol_type = other DNA
cagaagatccccggcaacgataattcgaccgccaccctgtgcctgggacaccacgccgtgccaaacggcaccatcgtgaagaccatcaccaacgaccgcatcgaggtgaccaatgccaccgagctggtgcagaacagctccatcggcgagatttgcggcagcccacaccagatcctggatggcggaaattgcaccctgattgatgccctgctgggcgatccccagtgcgacggcttccagaacaaggagtgggatctgttcgtggagcgctcgcgcgccaacagcaattgctacccctacgatgtgcccgactacgcctccctgcgctcgctggtggccagcagcggcaccctggagttcaagaacgagtccttcaattggaccggcgtgaagcagaatggcacctcctcggcctgcatccgcggcagcagcagcagcttcttcagccgcctgaactggctgaccagcctgaacaatatctacccggcccagaacgtgaccatgccaaataaggagcagttcgacaagctgtacatctggggcgtgcaccacccggataccgacaagaatcagatcagcctgttcgcccagtcctcgggccgcatcaccgtgtccaccaagcgctcgcagcaggccgtgatccccaacattggcagccgcccacgcatccgcgacatccccagccgcatctccatctactggaccatcgtgaagccaggcgacatcctgctgatcaacagcaccggcaatctgatcgccccacgcggctacttcaagatccgctccggcaagagcagcatcatgcgctcggatgcccccatcggcaagtgcaagagcgagtgcatcaccccgaacggctccatcccaaatgacaagcccttccagaacgtgaatcgcattacctacggcgcctgcccacgctacgtgaagcagtccaccctgaagctggccaccggaatgcgcaatgtgcccgagaagcagacccgcggcatcttcggagccattgccggcttcatcgagaatggctgggagggcatggtggatggctggtacggcttccgccaccagaactcggagggacgcggacaggccgccgatctgaagagcacccaggccgccatcgaccagatcaacggcaagctgaatcgcctgatcggcaagaccaacgagaagttccaccagatcgagaaggagttctcggaggtggagggacgcgtgcaggacctggagaagtacgtggaggataccaagatcgacctgtggagctacaatgccgagctgctggtggccctggagaaccagcacaccatcgatctgaccgactccgagatgaataagctgttcgagaagaccaagaagcagctgcgcgagaacgccgaggatatgggcaatggctgcttcaagatttaccacaagtgcgacaacgcctgcatcggctccatccgcaacgagacgtacgatcacaatgtgtaccgcgacgaggccctgaacaatcgcttccagatcaagggcgtggagctgaagtcgggctacaaggattggattctgtggatcagcttcgccatgtcctgcttcctgctgtgcatcgccctgctgggcttcatcatgggctcggctagcctggaggtgctgtttcagggaccaggatcccatcatcatcatcatcatcatcatcatcactaa

SEQ ID NO: 24 - protein, 560 aa, synthetic construct
QKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMGSASLEVLFQGPGSHHHHHHHHHH

SEQ ID NO: 25 - DNA, 1623 bp, synthetic construct
  source 1..1623: mol_type = other DNA
cagaagatccccggcaacgataattcgaccgccaccctgtgcctgggacaccacgccgtgccaaacggcaccatcgtgaagaccatcaccaacgaccgcatcgaggtgaccaatgccaccgagctggtgcagaacagctccatcggcgagatttgcggcagcccacaccagatcctggatggcggaaattgcaccctgattgatgccctgctgggcgatccccagtgcgacggcttccagaacaaggagtgggatctgttcgtggagcgctcgcgcgccaacagcaattgctacccctacgatgtgcccgactacgcctccctgcgctcgctggtggccagcagcggcaccctggagttcaagaacgagtccttcaattggaccggcgtgaagcagaatggcacctcctcggcctgcatccgcggcagcagcagcagcttcttcagccgcctgaactggctgaccagcctgaacaatatctacccggcccagaacgtgaccatgccaaataaggagcagttcgacaagctgtacatctggggcgtgcaccacccggataccgacaagaatcagatcagcctgttcgcccagtcctcgggccgcatcaccgtgtccaccaagcgctcgcagcaggccgtgatccccaacattggcagccgcccacgcatccgcgacatccccagccgcatctccatctactggaccatcgtgaagccaggcgacatcctgctgatcaacagcaccggcaatctgatcgccccacgcggctacttcaagatccgctccggcaagagcagcatcatgcgctcggatgcccccatcggcaagtgcaagagcgagtgcatcaccccgaacggctccatcccaaatgacaagcccttccagaacgtgaatcgcattacctacggcgcctgcccacgctacgtgaagcagtccaccctgaagctggccaccggaatgcgcaatgtgcccgagaagcagacccgcggcatcttcggagccattgccggcttcatcgagaatggctgggagggcatggtggatggctggtacggcttccgccaccagaactcggagggacgcggacaggccgccgatctgaagagcacccaggccgccatcgaccagatcaacggcaagctgaatcgcctgatcggcaagaccaacgagaagttccaccagatcgagaaggagttctcggaggtggagggacgcgtgcaggacctggagaagtacgtggaggataccaagatcgacctgtggagctacaatgccgagctgctggtggccctggagaaccagcacaccatcgatctgaccgactccgagatgaataagctgttcgagaagaccaagaagcagctgcgcgagaacgccgaggatatgggcaatggctgcttcaagatttaccacaagtgcgacaacgcctgcatcggctccatccgcaacgagacgtacgatcacaatgtgtaccgcgacgaggccctgaacaatcgcttccagatcaagggcgtggagctgaagtcgggctacaaggattggattctgtggatcagcttcgccatgtcctgcttcctgctgtgcatcgccctgctgggcttcatcatgggctcggctagctaa

SEQ ID NO: 26 - protein, 540 aa, synthetic construct
QKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMGSAS

SEQ ID NO: 27 - DNA, 1734 bp, synthetic construct
  source 1..1734: mol_type = other DNA
gatcgcatctgcaccggcatcaccagctccaactcgccgcacgtggtgaagaccgccacccagggcgaagtgaatgtgaccggcgtgatccccctgaccaccaccccaaccaagagccacttcgccaacctgaagggcaccgagacgcgcggcaagctgtgcccaaagtgcctgaattgcaccgatctggacgtggccctgggccgcccaaagtgcaccggcaagatcccatcggcccgcgtgtcgattctgcacgaggtgcgccccgtgacctccggctgcttcccgatcatgcacgatcgcaccaagattcgccagctgccaaatctgctgcgcggatacgagcacgtgcgcctgagcacccacaacgtgatcaatgccgaggatgccccaggccgcccctacgagatcggcacctcgggcagctgcccgaatatcaccaacggcaatggcttcttcgccacgatggcctgggccgtgccaaagaacaagaccgccaccaatcccctgaccatcgaggtgccgtacatctgcaccgagggcgaggatcagatcaccgtgtggggcttccacagcgacaacgagacgcagatggccaagctgtacggcgattccaagccccagaagttcaccagcagcgccaacggcgtgaccacccactacgtgtcccagatcggcggattcccaaatcagaccgaggatggcggcctgccacagtccggccgcatcgtggtggactacatggtgcagaagtcgggcaagaccggcaccatcacctaccagcgcggcatcctgctgccgcagaaagtgtggtgcgcctcgggccgcagcaaggtcatcaagggctcgctgcccctgattggagaggccgactgcctgcacgagaagtacggcggcctgaacaagagcaagccgtactacaccggcgagcacgccaaggccatcggcaactgcccaatctgggtgaagaccccgctgaagctggccaatggcaccaagtaccgcccacccgccaagctgctgaaggagcgcggcttcttcggagccattgccggcttcctggagggcggctgggagggaatgattgccggctggcacggctacacctcgcacggcgcccacggcgtggccgtggccgccgatctgaagagcacccaggaggccatcaataagatcaccaagaacctgaacagcctgtcggagctggaggtgaagaacctgcagcgcctgagcggcgctatggatgagctgcacaatgagatcctggagctggatgagaaggtggatgacctgcgcgccgacaccatctcctcgcagatcgagctggccgtgctgctgtcgaacgagggcatcatcaattcggaggatgagcacctgctggccctggagcgcaagctgaagaagatgctgggaccatcggccgtggagattggaaacggctgcttcgagacgaagcacaagtgcaatcagacctgcctggatcgcattgccgccggcaccttcgatgccggagagttcagcctgcccaccttcgattccctgaacatcaccgccgccagcctgaatgatgatggcctggacaatcacaccatcctgctgtactactcgacagccgccagctccctggccgtgaccctgatgatcgccatcttcgtggtgggcagcgctagcctggaggtgctgttccagggaccaggatcccatcatcatcatcatcatcatcatcatcactaa

SEQ ID NO: 28 - protein, 577 aa, synthetic construct
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVGSASLEVLFQGPGSHHHHHHHHHH

SEQ ID NO: 29 - DNA, 1674 bp, synthetic construct
  source 1..1674: mol_type = other DNA
gatcgcatctgcaccggcatcaccagctccaactcgccgcacgtggtgaagaccgccacccagggcgaagtgaatgtgaccggcgtgatccccctgaccaccaccccaaccaagagccacttcgccaacctgaagggcaccgagacgcgcggcaagctgtgcccaaagtgcctgaattgcaccgatctggacgtggccctgggccgcccaaagtgcaccggcaagatcccatcggcccgcgtgtcgattctgcacgaggtgcgccccgtgacctccggctgcttcccgatcatgcacgatcgcaccaagattcgccagctgccaaatctgctgcgcggatacgagcacgtgcgcctgagcacccacaacgtgatcaatgccgaggatgccccaggccgcccctacgagatcggcacctcgggcagctgcccgaatatcaccaacggcaatggcttcttcgccacgatggcctgggccgtgccaaagaacaagaccgccaccaatcccctgaccatcgaggtgccgtacatctgcaccgagggcgaggatcagatcaccgtgtggggcttccacagcgacaacgagacgcagatggccaagctgtacggcgattccaagccccagaagttcaccagcagcgccaacggcgtgaccacccactacgtgtcccagatcggcggattcccaaatcagaccgaggatggcggcctgccacagtccggccgcatcgtggtggactacatggtgcagaagtcgggcaagaccggcaccatcacctaccagcgcggcatcctgctgccgcagaaagtgtggtgcgcctcgggccgcagcaaggtcatcaagggctcgctgcccctgattggagaggccgactgcctgcacgagaagtacggcggcctgaacaagagcaagccgtactacaccggcgagcacgccaaggccatcggcaactgcccaatctgggtgaagaccccgctgaagctggccaatggcaccaagtaccgcccacccgccaagctgctgaaggagcgcggcttcttcggagccattgccggcttcctggagggcggctgggagggaatgattgccggctggcacggctacacctcgcacggcgcccacggcgtggccgtggccgccgatctgaagagcacccaggaggccatcaataagatcaccaagaacctgaacagcctgtcggagctggaggtgaagaacctgcagcgcctgagcggcgctatggatgagctgcacaatgagatcctggagctggatgagaaggtggatgacctgcgcgccgacaccatctcctcgcagatcgagctggccgtgctgctgtcgaacgagggcatcatcaattcggaggatgagcacctgctggccctggagcgcaagctgaagaagatgctgggaccatcggccgtggagattggaaacggctgcttcgagacgaagcacaagtgcaatcagacctgcctggatcgcattgccgccggcaccttcgatgccggagagttcagcctgcccaccttcgattccctgaacatcaccgccgccagcctgaatgatgatggcctggacaatcacaccatcctgctgtactactcgacagccgccagctccctggccgtgaccctgatgatcgccatcttcgtggtgggcagcgctagctaa

SEQ ID NO: 30 - protein, 557 aa, synthetic construct
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVGSAS

SEQ ID NO: 31 - protein, 566 aa, Alphainfluenzavirus influenzae
  source 1..566: note = Influenza A virus
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 32 - DNA, 114 bp, synthetic construct
  source 1..114: mol_type = other DNA
atgctcctcgtcaaccagtcacatcaaggcttcaacaaagaacatacctcaaaaatggtttccgctatcgtcctctacgtgctgctggctgctgctgcccactctgctttcgct

SEQ ID NO: 33 - DNA, 1815 bp, synthetic construct
  source 1..1815: mol_type = other DNA
gaattcgccgccaccatgctcctcgtcaaccagtcacatcaaggcttcaacaaagaacatacctcaaaaatggtttccgctatcgtcctctacgtgctgctggctgctgctgcccactctgctttcgctgacactctgtgcatcggataccacgctaacaactccaccgacactgtggacaccgtcctggagaagaacgtcaccgtgacccactctgtgaacctgctggaggacaagcacaacggcaagctgtgcaagctgagaggcgtggccccactgcacctgggcaagtgcaacatcgctggctggatactgggtaaccctgagtgcgaaagcctgtccactgctagatcatggtcctacatcgtggaaacttccaacagcgacaacggtacttgctacccaggagacttcatcaactacgaggagctgagggagcagctgtcatctgtgtccagcttcgaaaggttcgaaatcttcccaaagacttcatcttggcctaaccacgacagcgacaagggtgtgactgctgcctgccctcacgctggtgccaagtcattctacaagaacctgatctggctggtgaagaagggcaactcataccctaagctgaaccagacttacatcaacgacaagggcaaggaggtcctggtgctgtggggtatccaccaccctcccaccatcgctgcccaggagtctctgtaccagaacgctgacgcttacgtgttcgtcggtacttcacgctactccaagaagttcaagcccgaaatcgctactcgcccaaaggtgcgcgaccaggaaggcaggatgaactactactggactctggtggaacctggagacaagatcaccttcgaggctactggaaacctggtcgtgccaagatacgctttcactatggagcgtgacgctggttctggcatcatcatctccgacactcctgtccacgactgcaacaccacttgccagaccccagagggtgctatcaacacctctctgccattccagaacgtgcacccaatcaccatcggaaagtgccccaagtacgtgaagtctaccaagctgcgtctggctactggactgaggaacgtgccatctatccagtcacgcggtctgttcggtgctatcgctggcttcatcgagggcggttggactggcatggtggacggttggtacggataccaccaccagaacgagcagggctcaggttacgctgctgacctgaagtcaacccagaacgctatcgacaagatcactaacaaggtgaactctgtgatcgaaaagatgaacacccagttcaccgctgtgggaaaggagttcaaccacctggagaagcgtatcgagaacctgaacaagaaggtggacgacggattcctggacatctggacctacaacgctgagctgctggtcctgctggaaaacgagcgcaccctggactaccacgacagcaacgtcaagaacctgtacgagaaggtgcgcaaccagctgaagaacaacgctaaggagatcggcaacggctgcttcgagttctaccacaagtgcgacaacacttgcatggaatccgtgaagaacggtacttacgactacccaaagtactctgaggaggccaagctgaacagggagaagatcgacggcgtgaagctggagtccactcgcatctaccagatcctggctatctactcaactgtggcctcttcactggtgctggtcgtgtctctgggtgctatcagcttctggatgggttcagctggtctcgaagttttgttccagggaccccatcaccaccatcatcaccaccaccatcactaaaagctt

SEQ ID NO: 34 - DNA, 24 bp, synthetic construct
  source 1..24: mol_type = other DNA
ctcgaagttttgttccagggaccc

SEQ ID NO: 35 - protein, 8 aa, synthetic construct
LEVLFQGP

SEQ ID NO: 36 - DNA, 30 bp, synthetic construct
  source 1..30: mol_type = other DNA
catcaccaccatcatcaccaccaccatcac

SEQ ID NO: 37 - protein, 10 aa, synthetic construct
HHHHHHHHHH

(19) United States
(12) Patent Application Publication
(10) Pub. No.: US 2025/0325649 A1
(43) Pub. Date: Oct. 23, 2025

(54) POLYPEPTIDE FRAGMENTS, IMMUNOGENIC COMPOSITION AGAINST INFLUENZA VIRUS, AND IMPLEMENTATIONS THEREOF

(71) Applicant: MYNVAX PRIVATE LIMITED, Bangalore (IN)

(72) Inventors: Raghavan VARADARAJAN, Bangalore (IN); Suman PANDEY, Bangalore (IN); Nupur AGARWAL, Bangalore (IN); Aditya UPADHYAYA, Bangalore (IN)

(21) Appl. No.: 18/725,107

(22) PCT Filed: Dec. 30, 2022
(86) PCT No.: PCT/IN2022/051149

§ 371 Date: Jun. 27, 2024

(30) Foreign Application Priority Data
Dec. 31, 2021 (IN) 202141062288

(51) Int. Cl. A61K 39/145; A61K 39/00; A61P 37/04; C07K 14/005

(52) U.S. Cl. CPC A61K 39/145; A61P 37/04; C07K 14/005; A61K 2039/55566; A61K 2039/70; C07K 2319/02; C12N 2760/16122; C12N 2760/16134; C12N 2760/16171; C12N 2760/16222; C12N 2760/16234; C12N 2760/16271

(57) ABSTRACT

The present disclosure provides a polypeptide fragment comprising a polypeptide of a modified hemagglutinin protein, wherein said modified hemagglutinin protein comprises a linker peptide replacing the polypeptide in the cytoplasmic domain of hemagglutinin protein, wherein the hemagglutinin protein is obtained from at least one seasonal strain of influenza virus, wherein said polypeptide of the modified hemagglutinin protein is having at least 80%, or at least 90%, or at least 95%, or at least 97% sequence identity to at least one sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

### PRIORITY AND CROSS REFERENCE TO RELATED APPLICATIONS

This application is the U.S. National Stage Application under 35 U.S.C. § 371 of International Application No. PCT/1N2022/051149, filed Dec. 30, 2022, designating U.S., and published in English as WO 2023/126982 A1 on Jul. 6, 2023, which claims the benefit of Indian Patent Application No. IN 202141062288, filed Dec. 31, 2021. Any and all applications for which a foreign or a domestic priority is claimed is/are identified in the Application Data Sheet filed herewith and is/are hereby incorporated by reference in their entirety under 37 C.F.R. § 1.57.

### SEQUENCE LISTING IN ELECTRONIC FORMAT

The present application is being filed along with an Electronic Sequence Listing as an XML file in ST.26 format via Patent Center. The Electronic Sequence Listing is provided as a file entitled PD045263IN-SC-sequence listing.xml, created on Dec. 26, 2022, which is 68,831 bytes in size, which is replaced by a Replacement Electronic Sequence Listing provided as a file entitled PD045263IN-SC-replacement sequence listing.xml, created on Feb. 23, 2025, which is 69,102 bytes in size. The information in the Electronic Sequence Listing is incorporated herein by reference in its entirety.

### FIELD OF INVENTION

The present disclosure broadly relates to the field of immunobiology, and particularly discloses polypeptides, and immunogenic composition for eliciting immune response against influenza.

### BACKGROUND OF INVENTION

Influenza viruses are members of the Orthomyxoviridae family and considered to be one of the most ubiquitous viruses present in the world, affecting both humans and livestock. Influenza results in an economic burden, morbidity and even mortality, which are significant. The influenza viruses are negative-sense, single-stranded segmented RNA viruses. It consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material. Influenza virus comprises two surface antigens, glycoproteins neuraminidase (NA) and haemagluttinin (HA), which appear as spikes, 10 to 12 nm long, at the surface of the particles. It is these surface proteins, particularly the haemagluttinin that determine the antigenic specificity of the influenza subtypes. Virus strains are classified according to host species of origin, geographic site and year of isolation, serial number. There are three genera of influenza viruses (A, B, and C) that are divided based on antigenic differences in the viral nucleoprotein (NP) and matrix protein (M). Influenza A viruses are further divided based on the antigenic properties of their surface glycoproteins into 16 HA subtypes (H1-H16) and nine NA subtypes (N1-N9) (Yoon S W, et cal. Evolution and ecology of influenza A viruses. Influenza Pathogenesis and Control-Volume I. 2014:359-75.) Viruses of all HA and NA subtypes have been recovered from aquatic birds, but only three HA subtypes (H1, H2, and H3) and two NA subtypes (N1 and N2) have established stable lineages in the human population since 1918. Only one subtype of HA and one of NA are recognised for influenza B viruses.

Infection with an influenza virus has results in a high morbidity, disability and mortality burdens worldwide. Seasonal influenza viruses (A H1N1, A H3N2 and two B lineages) cause recurring annual epidemics, with an estimated 3 to 5 million cases of severe illness and up to 650,000 deaths every year (Organization, W. H. Factsheet on seasonal influenza. (2018)). Individuals at risk of developing severe disease and complications, such as viral and (secondary) bacterial pneumonia and cardio-vascular disease, include the very young (below one year of age), older adults above 65, pregnant women, and individuals with underlying (chronic) illnesses, such as metabolic, respiratory and cardiac conditions. Influenza pandemics can yield devastating morbidity and mortality burdens, including in otherwise healthy children and young adults (which are typically spared from severe disease during seasonal epidemics) (Taubenberger, J. K. & Morens, D. M. 1918 Influenza: The mother of all pandemics. Emerging Infectious Diseases (2006). doi:10.3201/eid1209.05-0979). The most preferred and cost-effective intervention tool currently available to prevent influenza virus infection and disease is vaccination.

The currently available licensed influenza vaccines include inactivated whole or split viruses, (recombinant) viral subunit, and live attenuated vaccines, and contain viral strains or HAs closely related to the putative upcoming seasonal strains of H1N1 and H3N2 influenza A viruses, as well as (from 2009-2010 season onwards) both antigenic lineages of the influenza B viruses. Although the currently available licensed influenza vaccine effectively reduces influenza incidence, and associated disease severity and mortality among the young adults, however, their effectiveness remains incomplete, especially in the major at-risk groups. For example, in older adults, seasonal influenza vaccines reduce confirmed influenza virus infection by 50%-60%, typical influenza-like illness by 40%-50% and influenza-related complications by 30%-50% (Treanor, J. & Falsey, A. Respiratory viral infections in the elderly. Antiviral Research (1999). doi:10.1016/S0166-3542(99)00062-5; Beyer, W. E. P. et al. Cochrane re-arranged: Support for policies to vaccinate elderly people against influenza. Vaccine (2013). doi:10.1016/j.vaccine.2013.09.063). Further, the current vaccines require annual evaluation and reformulation because of the unremitting antigenic drift of seasonal influenza viruses, and the escape of drift variants from pre-existing immunity elicited by previous infections or vaccination. Hence, this process requires vaccine strains to be updated every year. Furthermore, mismatch between the vaccine and circulating strains can result in dramatic reduction of vaccine protective efficacy. Additionally, most of the licensed vaccines are produced using embryonated hen's eggs, which further results in antigenic changes associated with viral adaptation to replication in the chorioallantoic membrane. These changes may also reduce vaccine match with circulating strains, and consequently reduce vaccine protective effectiveness (Skowronski, D. M. et al. Low 2012-13 influenza vaccine effectiveness associated with mutation in the egg-adapted H3N2 vaccine strain not antigenic drift in circulating viruses. PLoS One (2014). doi:10.1371/journal.pone.0092153).

Thus, relatively long production times of influenza vaccines using embryonated eggs and limited scalability in situations of increased need, remain major obstacles to pandemic preparedness against influenza viruses.

Accordingly, there is a dire need in the art to develop an effective, cost-effective, influenza vaccine composition with better immunogenicity that can elicit immune responses against multiple influenza strains with pandemic potential.

### SUMMARY OF THE INVENTION

In an aspect of the present disclosure, there is provided a polypeptide fragment comprising a polypeptide derived from different strains of influenza virus that is recommended by WHO or any future or past seasonal influenza vaccine strains recommended by WHO, wherein the polypeptide is modified by deleting and replacing the cytoplasmic domain of Hemagglutinin (HA) with a linker.

In an aspect of the present disclosure, there is provided a polypeptide fragment comprising a polypeptide of a modified Hemagglutinin (HA) protein, wherein said modified protein comprises a linker peptide replacing the polypeptide fragment in the cytoplasmic domain of HA protein, wherein the hemagglutinin protein is obtained from at least one or a plurality of strains of influenza virus.

A polypeptide fragment comprising a polypeptide of a modified hemagglutinin protein, wherein said modified hemagglutinin protein comprises a linker peptide replacing the polypeptide in the cytoplasmic domain of hemagglutinin protein, wherein the hemagglutinin protein is obtained from at least one strain of influenza virus, wherein said polypeptide of the modified hemagglutinin protein is having at least 80%, or at least 90%, or at least 95%, or at least 97% sequence identity to at least one sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an aspect of the present disclosure, there is provided a polypeptide fragment comprising a polypeptide consisting of at least one polypeptide having an amino acid selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In another aspect of the present disclosure, there is provided a nucleic acid fragment encoding the polypeptide fragment as described herein.

In another aspect of the present disclosure, there is provided a recombinant construct comprising a nucleic acid fragment encoding a polypeptide fragment as described herein, operably linked to a promoter, and a nucleic acid sequence encoding a signal peptide.

In another aspect of the present disclosure, there is provided a recombinant vector comprising the recombinant construct as described herein.

In another aspect of the present disclosure, there is provided a recombinant host cell comprising the recombinant vector as described herein or the recombinant construct as described herein.

In another aspect of the present disclosure, there is provided an immunogenic composition comprising a combination of four polypeptide fragments having at least 80%, or at least 90%, or at least 95%, or at least 97% sequence identity to a polypeptide having an amino acid sequence as set forth in SEQ ID NO: 10, SEQ ID NO: 12, SEQ ID NO: 14, SEQ ID NO: 16, a pharmaceutically acceptable carrier.

In another aspect of the present disclosure, there is provided an immunogenic composition comprising a combination of four polypeptide fragment having at least 80%, or at least 90%, or at least 95%, or at least 97% sequence identity to a polypeptide having an amino acid sequence as set forth in SEQ ID NO: 14, SEQ ID NO: 16, SEQ ID NO: 18, and SEQ ID NO: 20, a pharmaceutically acceptable carrier.

In another aspect of the present disclosure, there is provided a method for obtaining the immunogenic composition as described herein, wherein the method comprises: (a) culturing the recombinant host cell as described herein under suitable conditions to obtain the polypeptide fragment as described herein; (b) subjecting the polypeptide to purification; and (c) contacting the polypeptide of step (b) with a pharmaceutically acceptable carrier for obtaining the immunogenic composition.

In another aspect of the present disclosure, there is provided a method for eliciting an immune response to an influenza disease in a subject, the method comprising administering the subject a pharmaceutically effective amount of the immunogenic composition as described herein.

In another aspect of the present disclosure, there is provided a method for preventing an influenza disease in a subject, the method comprising administering the subject a pharmaceutically effective amount of the immunogenic composition as described herein.

In another aspect of the present disclosure, there is provided a kit comprising the polypeptide as described herein, or the immunogenic composition as described herein, and an instruction leaflet.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the domain organization of hemagglutinin (HA) protein, in accordance with an embodiment of the present disclosure.

FIG. 2 depicts the ELISA titer values in mice immunized with the test immunogenic composition (NQ20) (SEQ ID NO: 10+SEQ ID NO: 12+SEQ ID NO: 14+SEQ ID NO: 16) adjuvanted with: (A) AddaVax™; and (B) SWE adjuvant, in accordance with an embodiment of the present disclosure.

FIG. 3A depicts the comparison of ELISA titer values in guinea pigs immunized with the test immunogenic composition (NQ20) (adjuvanted with AddaVax™) and Influvac (an egg-grown, inactivated viral vaccine containing full-length HA protein having cytoplasmic domain), respectively, in accordance with an embodiment of the present disclosure.

FIG. 3B depicts the ELISA titer values in guinea pigs immunized with the test immunogenic composition (NQ20) adjuvanted with SWE adjuvant, in accordance with an embodiment of the present disclosure. Doses of NQ21 and Influvac are indicated.

FIG. 4 depicts the comparison of ELISA titer values in guinea pigs immunized with the test immunogenic composition (NQ21) (SEQ ID NO: 14, SEQ ID NO: 16, SEQ ID NO: 18, SEQ ID NO: 20) (adjuvanted with SWE adjuvant) and Influvac, respectively, wherein FIG. 4A represents H1N1, FIG. 4B represents H3N2, FIG. 4C represents Flu B Victoria, and FIG. 4D represents Flu B Yamagata, in accordance with an embodiment of the present disclosure.

FIG. 5 depicts the results of immunogenic studies (HI titers) of the test composition (NQ21), according to embodiments herein, in ferrets. The immune response was studied against four influenza strains, wherein FIG. 5A represents H1N1, FIG. 5B represents H3N2, FIG. 5C represents Flu B Victoria, and FIG. 5D represents Flu B Yamagata. Doses of NQ21 and Influvac are indicated, in accordance with an embodiment of the present disclosure.

FIG. 6 depicts the results of immunogenic studies (HI titers) of the test composition (NQ21), according to embodiments herein, in hamster. The immune response was studied against four influenza strains, wherein FIG. 6A represents H1N1, FIG. 6B represents H3N2, FIG. 6C represents Flu B Victoria, and FIG. 6D represents Flu B Yamagata. Doses of NQ21 and Influvac are indicated, in accordance with an embodiment of the present disclosure.

FIG. 7 depicts the results of immunogenic studies (HI titers) in ferrets, on day 2 (pre), day 0 (prime) and day 21 (boost), with quadrivalent NQ21 antigens at 15 μg+SWE and Influvac, 15 μg each of the inactivated virus (quadrivalent). The immune response was studied against four influenza strains, wherein FIG. 7A represents H1N1, FIG. 7B represents H3N2, FIG. 7C represents Flu B Victoria, and FIG. 7D represents Flu B Yamagata. Dose of NQ21 and Influvac are indicated, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### SEQUENCES USED IN THE PRESENT DISCLOSURE

SEQ ID NO: 1 depicts the nucleic acid sequence encoding a polypeptide expressed by a host cell transfected with sMH1_02HA0d

GACACTCTGTGCATCGGATACCACGCTAACAACTCCACCGACACTGTGG
ACACCGTCCTGGAGAAGAACGTCACCGTGACCCACTCTGTGAACCTGCT
GGAGGACAAGCACAACGGCAAGCTGTGCAAGCTGAGAGGCGTGGCCCC
ACTGCACCTGGGCAAGTGCAACATCGCTGGCTGGATACTGGGTAACCCT
GAGTGCGAAAGCCTGTCCACTGCTAGATCATGGTCCTACATCGTGGAAA
CTTCCAACAGCGACAACGGTACTTGCTACCCAGGAGACTTCATCAACTA
CGAGGAGCTGAGGGAGCAGCTGTCATCTGTGTCCAGCTTCGAAAGGTT
CGAAATCTTCCCAAAGACTTCATCTTGGCCTAACCACGACAGCGACAAG
GGTGTGACTGCTGCCTGCCCTCACGCTGGTGCCAAGTCATTCTACAAGA
ACCTGATCTGGCTGGTGAAGAAGGGCAACTCATACCCTAAGCTGAACC
AGACTTACATCAACGACAAGGGCAAGGAGGTCCTGGTGCTGTGGGGTA
TCCACCACCCTCCCACCATCGCTGCCCAGGAGTCTCTGTACCAGAACGC
TGACGCTTACGTGTTCGTCGGTACTTCACGCTACTCCAAGAAGTTCAAG
CCCGAAATCGCTACTCGCCCAAAGGTGCGCGACCAGGAAGGCAGGATG
AACTACTACTGGACTCTGGTGGAACCTGGAGACAAGATCACCTTCGAG
GCTACTGGAAACCTGGTCGTGCCAAGATACGCTTTCACTATGGAGCGTG
ACGCTGGTTCTGGCATCATCATCTCCGACACTCCTGTCCACGACTGCAA
CACCACTTGCCAGACCCCAGAGGGTGCTATCAACACCTCTCTGCCATTC
CAGAACGTGCACCCAATCACCATCGGAAAGTGCCCCAAGTACGTGAAG
TCTACCAAGCTGCGTCTGGCTACTGGACTGAGGAACGTGCCATCTATCC
AGTCACGCGGTCTGTTCGGTGCTATCGCTGGCTTCATCGAGGGCGGTTG
GACTGGCATGGTGGACGGTTGGTACGGATACCACCACCAGAACGAGCA
GGGCTCAGGTTACGCTGCTGACCTGAAGTCAACCCAGAACGCTATCGA
CAAGATCACTAACAAGGTGAACTCTGTGATCGAAAAGATGAACACCCA
GTTCACCGCTGTGGGAAAGGAGTTCAACCACCTGGAGAAGCGTATCGA
GAACCTGAACAAGAAGGTGGACGACGGATTCCTGGACATCTGGACCTA
CAACGCTGAGCTGCTGGTCCTGCTGGAAAACGAGCGCACCCTGGACTA
CCACGACAGCAACGTCAAGAACCTGTACGAGAAGGTGCGCAACCAGCT
GAAGAACAACGCTAAGGAGATCGGCAACGGCTGCTTCGAGTTCTACCA
CAAGTGCGACAACACTTGCATGGAATCCGTGAAGAACGGTACTTACGA
CTACCCAAAGTACTCTGAGGAGGCCAAGCTGAACAGGGAGAAGATCGA
CGGCGTGAAGCTGGAGTCCACTCGCATCTACCAGATCCTGGCTATCTAC
TCAACTGTGGCCTCTTCACTGGTGCTGGTCGTGTCTCTGGGTGCTATCA
GCTTCTGGATGGGTTCAGCTGGTCTCGAAGTTTTGTTCCAGGGACCCCA
TCACCACCATCATCACCACCACCATCACTAA

SEQ ID NO: 2 depicts the amino acid sequence of a polypeptide expressed by the host cell transfected with sMH1_02HA0d

DTLCIGYHANNSTDTVDTVLEKNVTVTHSVNLLEDKHNGKLCKLRGVAPLH
LGKCNIAGWILGNPECESLSTARSWSYIVETSNSDNGTCYPGDFINYEELR
EQLSSVSSFERFEIFPKTSSWPNHDSDKGVTAACPHAGAKSFYKNLIWLVK
KGNSYPKLNQTYINDKGKEVLVLWGIHHPPTIAAQESLYQNADAYVFVGT
SRYSKKFKPEIATRPKVRDQEGRMNYYWTLVEPGDKITFEATGNLVVPRYA
FTMERDAGSGIIISDTPVHDCNTTCQTPEGAINTSLPFQNVHPITIGKCPK
YVKSTKLRLATGLRNVPSIQSRGLFGAIAGFIEGGWTGMVDGWYGYHHQNE
QGSGYAADLKSTQNAIDKITNKVNSVIEKMNTQFTAVGKEFNHLEKRIENL
NKKVDDGFLDIWTYNAELLVLLENERTLDYHDSNVKNLYEKVRNQLKNN
AKEIGNGCFEFYHKCDNTCMESVKNGTYDYPKYSEEAKLNREKIDGVKLE
STRIYQILAIYSTVASSLVLVVSLGAISFWMGSAGLEVLFQGPHHHHHHHH
HH*

SEQ ID NO: 3 depicts the nucleic acid sequence encoding a polypeptide expressed by a host cell transfected with sMH3_02HA0d

CAGAAGATCCCCGGCAACGACAACTCAACCGCTACTCTGTGCCTGGGA
CACCACGCCGTGCCAAACGGCACCATCGTCAAGACCATCACTAACGAC
CGTATCGAGGTGACCAACGCTACTGAACTGGTCCAGAACTCCAGCATC
GGAGAAATCTGCGACTCCCCACACCAGATCCTGGACGGTGGCAACTGC
ACTCTGATCGACGCTCTGCTGGGCGACCCTCAGTGCGACGGATTCCAGA
ACAAGAAGTGGGACCTGTTCGTGGAACGCTCTCGTGCCTACTCAAACTG
CTACCCTTACGACGTGCCCGACTACGCTTCTCTGAGGTCACTGGTCGCC
TCTTCAGGAACCCTGGAGTTCAAGAACGAATCTTTCAACTGGGCTGGTG
TCACTCAGAACGGCAAGTCCTTCAGCTGCATCCGTGGTTCCAGCTCTTC
ATTCTTCTCAAGGCTGAACTGGCTGACCCACCTGAACTACACTTACCCC
GCCCTGAACGTGACCATGCCAAACAAGGAGCAGTTCGACAAGCTGTAC
ATCTGGGGAGTCCACCACCCCGGTACTGACAAGGACCAGATCAGCCTG
TACGCTCAGTCCAGCGGTAGAATCACCGTGTCCACTAAGCGCAGCCAG
CAGGCCGTCATCCCCAACATCGGCAGCAGGCCAAGAATCCGCGACATC
CCTTCCAGAATCAGCATCTACTGGACCATCGTGAAGCCAGGCGACATCC
TGCTGATCAACTCCACTGGCAACCTGATCGCTCCTCGTGGATACTTCAA
GATCAGGTCCGGCAAGTCCTCCATCATGCGCAGCGACGCCCCTATCGGA
AAGTGCAAGTCTGAGTGCATCACCCCCAACGGTTCAATCCCAAACGAC
AAGCCTTTCCAGAACGTGAACCGTATCACTTACGGCGCTTGCCCTCGTT
ACGTCAAGCAGAACACCCTGAAGCTGGCCACTGGAATGAGAAACGTGC
CCGAAAAGCAGACCCGCGGTATCTTCGGCGCTATCGCCGGCTTCATCGA
GAACGGATGGGAGGGTATGGTGGACGGCTGGTACGGATTCCGTCACCA
GAACTCCGAGGGAAGGGGTCAGGCTGCCGACCTGAAGAGCACCCAGGC
TGCCATCGACCAGATCAACGGCAAGCTGAACAGACTGATCGGAAAGAC
TAACGAAAAGTTCCACCAGATCGAGAAGGAGTTCAGCGAGGTGGAAGG
TCGCGTCCAGGACCTGGAGAAGTACGTGGAGGACACCAAGATCGACCT
GTGGTCCTACAACGCTGAGCTGCTGGTCGCCCTGGAAAACCAGCACAC
CATCGACCTGACTGACAGCGAGATGAACAAGCTGTTCGAAAAGACCAA
GAAGCAGCTGCGTGAGAACGCTGAGGACATGGGCAACGGATGCTTCAA
GATTTACCACAAGTGCGACAACGCCTGCATCGGTTCTATCAGGAACGA
GACTTACGACCACAACGTGTACAGAGACGAAGCTCTGAACAACCGCTT
CCAGATCAAGGGTGTCGAGCTGAAGTCAGGCTACAAGGACTGGATCTT
GTGGATCTCTTTCGCCATCTCATGCTTCCTGCTGTGCGTGGCTCTGCTGG
GTTTCATCATGGGTTCAGCTGGTCTCGAAGTTCTGTTCCAAGGACCGCA
CCATCATCACCATCACCATCATCATCACTAA

SEQ ID NO: 4 depicts the amino acid sequence of a polypeptide expressed by the host cell transfected with sMH3_02HA0d

QKIPGNDNSTATLCLGHHAVPNGTIVKTITNDRIEVTNATELVQNSSIGE
ICDSPHQILDGGNCTLIDALLGDPQCDGFQNKKWDLFVERSRAYSNCYPY
DVPDYASLRSLVASSGTLEFKNESFNWAGVTQNGKSFSCIRGSSSSFFSR
LNWLTHLNYTYPALNVTMPNKEQFDKLYIWGVHHPGTDKDQISLYAQSSG
RITVSTKRSQQAVIPNIGSRPRIRDIPSRISIYWTIVKPGDILLINSTGN
LIAPRGYFKIRSGKSSIMRSDAPIGKCKSECITPNGSIPNDKPFQNVNRI
TYGACPRYVKQNTLKLATGMRNVPEKQTRGIFGAIAGFIENGWEGMVDGW
YGFRHQNSEGRGQAADLKSTQAAIDQINGKLNRLIGKTNEKFHQIEKEFS
EVEGRVQDLEKYVEDTKIDLWSYNAELLVALENQHTIDLTDSEMNKLFEK
TKKQLRENAEDMGNGCFKIYHKCDNACIGSIRNETYDHNVYRDEALNNRF
QIKGVELKSGYKDWILWISFAISCFLLCVALLGFIMGSAGLEVLFQGPHH
HI*

SEQ ID NO: 5 depicts the nucleic acid sequence encoding a polypeptide expressed by a host cell transfected with sMHV_02HA0d

GACAGGATCTGCACCGGTATCACTTCCAGCAACAGCCCCCACGTGGTCA
AGACTGCTACCCAGGGAGAAGTGAACGTCACTGGTGTGATCCCCCTGA
CCACTACCCCAACCAAGTCTCACTTCGCCAACCTGAAGGGCACTGAAAC
CCGCGGCAAGCTGTGCCCAAAGTGCCTGAACTGCACTGACCTGGACGT
GGCTCTGGGCAGACCCAAGTGCACCGGAAAGATCCCATCCGCCCGCGT
CAGCATCCTGCACGAAGTGCGTCCTGTCACTTCTGGCTGCTTCCCCATC
ATGCACGACCGCACCAAGATCCGTCAGCTGCCTAACCTGCTGAGGGGTT
ACGAGCACGTGAGACTGTCAACTCACAACGTCATCAACGCTGAGGACG
CCCCAGGCCGCCCTTACGAGATCGGCACCTCCGGCAGCTGCCCCAACAT
CACTAACGGAAACGGTTTCTTCGCTACTATGGCTTGGGCCGTCCCAAAG
AACAAGACTGCCACCAACCCTCTGACCATCGAAGTGCCCTACATCTGCA
CCGAGGGCGAGGACCAGATCACTGTCTGGGGATTCCACTCCGACAACG
AGACTCAGATGGCTAAGCTGTACGGCGACAGCAAGCCTCAGAAGTTCA
CCTCTTCAGCCAACGGAGTGACTACCCACTACGTCTCCCAGATCGGTGG
CTTCCCAAACCAGACCGAGGACGGAGGTCTGCCTCAGTCTGGTCGTATC
GTGGTGGACTACATGGTGCAGAAGTCAGGAAAGACTGGCACCATCACT
TACCAGCGCGGAATCCTGCTGCCTCAGAAAGTGTGGTGCGCTTCTGGTC
GTTCAAAGGTCATCAAGGGTTCCCTGCCCCTGATCGGTGAAGCTGACTG
CCTGCACGAGAAGTACGGCGGACTGAACAAGAGCAAGCCCTACTACAC
TGGAGAACACGCTAAGGCCATCGGTAACTGCCCAATCTGGGTCAAGAC
TCCTCTGAAGCTGGCTAACGGCACCAAGTACAGGCCTCCCGCCAAGCTG
CTGAAGGAGAGAGGCTTCTTCGGAGCTATCGCCGGTTTCCTGGAAGGTG
GCTGGGAGGGCATGATCGCTGGTTGGCACGGCTACACCTCCCACGGTG
CTCACGGTGTGGCTGTGGCTGCCGACCTGAAGTCAACTCAGGAAGCCAT
CAACAAGATCACCAAGAACCTGAACTCTCTGTCAGAGCTGGAAGTGAA
GAACCTGCAACGCCTGTCCGGAGCTATGGACGAGCTGCACAACGAGAT
CCTGGAACTGGACGAGAAGGTGGACGACCTGCGTGCTGACACCATCTC
CAGCCAGATCGAACTGGCCGTCCTGCTGTCCAACGAGGGAATCATCAA
CAGCGAGGACGAACACCTGCTGGCTCTGGAAAGGAAGCTGAAGAAGAT
GCTGGGTCCAAGCGCCGTGGAAATCGGCAACGGATGCTTCGAAACCAA
GCACAAGTGCAACCAGACTTGCCTGGACAGAATCGCTGCCGGTACTTTC
GACGCTGGCGAGTTCTCTCTGCCTACCTTCGACTCACTGAACATCACTG
CTGCCTCTCTGAACGACGACGGCCTGGACAACCACACCATCCTGCTGTA
CTACTCAACTGCTGCCTCTTCACTGGCTGTCACCCTGATGATCGCCATCT
TCGTGGTCGGTTCAGCTGGTCTCGAAGTTCTGTTCCAGGGTCCGCACCA
CCATCACCATCACCACCACCATCACTAA

SEQ ID NO: 6 depicts the amino acid sequence of a polypeptide expressed by the host cell transfected with sMHV_02HA0d

DRICTGITSSNSPHVVKTATQGEVNVTGVIPLTTTPTKSHFANLKGTETR
GKLCPKCLNCTDLDVALGRPKCTGKIPSARVSILHEVRPVTSGCFPIMHD
RTKIRQLPNLLRGYEHVRLSTHNVINAEDAPGRPYEIGTSGSCPNITNGN
GFFATMAWAVPKNKTATNPLTIEVPYICTEGEDQITVWGFHSDNETQMAK
LYGDSKPQKFTSSANGVTTHYVSQIGGFPNQTEDGGLPQSGRIVVDYMVQ
KSGKTGTITYQRGILLPQKVWCASGRSKVIKGSLPLIGEADCLHEKYGGL
NKSKPYYTGEHAKAIGNCPIWVKTPLKLANGTKYRPPAKLLKERGFFGAI
AGFLEGGWEGMIAGWHGYTSHGAHGVAVAADLKSTQEAINKITKNLNSLS
ELEVKNLQRLSGAMDELHNEILELDEKVDDLRADTISSQIELAVLLSNEG
IINSEDEHLLALERKLKKMLGPSAVEIGNGCFETKHKCNQTCLDRIAAGT
FDAGEFSLPTFDSLNITAASLNDDGLDNHTILLYYSTAASSLAVTLMIAI
FVVGSAGLEVLFQGPHHHHHHHHHH*

SEQ ID NO: 7 depicts the nucleic acid sequence encoding a polypeptide expressed by a host cell transfected with sMHY_01HA0d

GACAGGATCTGCACTGGTATCACCTCCAGCAACTCACCTCACGTGGTCA
AGACTGCTACCCAGGGTGAAGTGAACGTCACCGGCGTGATCCCCCTGA
CCACTACCCCAACTAAGTCCTACTTCGCCAACCTGAAGGGAACTAGGAC
CCGCGGCAAGCTGTGCCCTGACTGCCTGAACTGCACCGACCTGGACGTG
GCTCTGGGAAGGCCCATGTGCGTCGGTACTACCCCATCCGCTAAGGCCA
GCATCCTGCACGAGGTGCGCCCTGTCACCTCTGGCTGCTTCCCCATCAT
GCACGACAGGACTAAGATCAGACAGCTGCCAAACCTGCTGCGCGGATA
CGAAAAGATCCGTCTGTCAACTCAGAACGTCATCGACGCTGAGAAGGC
CCCAGGTGGCCCTTACCGCCTGGGCACTTCCGGAAGCTGCCCTAACGCT
ACCTCCAAGATCGGCTTCTTCGCCACTATGGCTTGGGCCGTGCCCAAGG
ACAACTACAAGAACGCCACTAACCCTCTGACCGTGGAAGTCCCCTACAT
CTGCACTGAGGGAGAGGACCAGATCACCGTCTGGGGTTTCCACAGCGA
CAACAAGACCCAGATGAAGTCTCTGTACGGAGACTCAAACCCTCAGAA
GTTCACTTCTTCAGCTAACGGTGTGACTACCCACTACGTCTCTCAGATC
GGCGACTTCCCAGACCAGACCGAGGACGGAGGTCTGCCTCAGTCAGGT
CGTATCGTGGTGGACTACATGATGCAGAAGCCTGGCAAGACCGGCACC
ATCGTGTACCAGCGCGGAGTCCTGCTGCCACAGAAAGTGTGGTGCGCTT
CTGGTCGTTCAAAGGTCATCAAGGGCTCCCTGCCACTGATCGGAGAAGC
CGACTGCCTGCACGAGGAATACGGCGGACTGAACAAGAGCAAGCCTTA
CTACACCGGCAAGCACGCTAAGGCCATCGGCAACTGCCCAATCTGGGT
CAAGACCCCTCTGAAGCTGGCTAACGGCACTAAGTACAGGCCTCCCGC
CAAGCTGCTGAAGGAGAGAGGATTCTTCGGTGCTATCGCTGGCTTCCTG
GAAGGTGGCTGGGAGGGAATGATCGCTGGCTGGCACGGATACACCTCC
CACGGTGCTCACGGCGTGGCTGTGGCTGCCGACCTGAAGAGCACCCAG
GAAGCCATCAACAAGATCACTAAGAACCTGAACTCTCTGTCAGAGCTG
GAAGTGAAGAACCTGCAACGCCTGAGCGGAGCTATGGACGAGCTGCAC
AACGAGATCCTGGAACTGGACGAGAAGGTGGACGACCTGCGTGCTGAC
ACCATCTCCAGCCAGATCGAACTGGCCGTCCTGCTGTCTAACGAGGGTA
TCATCAACTCAGAGGACGAACACCTGCTGGCTCTGGAAAGGAAGCTGA
AGAAGATGCTGGGTCCTTCCGCTGTGGACATCGGAAACGGTTGCTTCGA
GACTAAGCACAAGTGCAACCAGACCTGCCTGGACAGAATCGCTGCCGG
CACCTTCAACGCTGGAGAGTTCTCCCTGCCAACTTTCGACAGCCTGAAC
ATCACCGCTGCCTCCCTGAACGACGACGGTCTGGACAACCACACTATCC
TGCTGTACTACAGCACCGCTGCCTCTTCACTGGCTGTGACTCTGATGCT
GGCCATCTTCATCGTGGGTTCAGCTGGTCTCGAAGTTCTGTTCCAGGGT
CCCCATCACCACCATCATCACCATCACCACCACTAA

SEQ ID NO: 8 depicts the amino acid sequence of a polypeptide expressed by the host cell transfected with sMHY_01HA0d

DRICTGITSSNSPHVVKTATQGEVNVTGVIPLTTTPTKSYFANLKGTRT
RGKLCPDCLNCTDLDVALGRPMCVGTTPSAKASILHEVRPVTSGCFPIM
HDRTKIRQLPNLLRGYEKIRLSTQNVIDAEKAPGGPYRLGTSGSCPNAT
SKIGFFATMAWAVPKDNYKNATNPLTVEVPYICTEGEDQITVWGFHSDN
KTQMKSLYGDSNPQKFTSSANGVTTHYVSQIGDFPDQTEDGGLPQSGRI
VVDYMMQKPGKTGTIVYQRGVLLPQKVWCASGRSKVIKGSLPLIGEADC
LHEEYGGLNKSKPYYTGKHAKAIGNCPIWVKTPLKLANGTKYRPPAKLL
KERGFFGAIAGFLEGGWEGMIAGWHGYTSHGAHGVAVAADLKSTQEAIN
KITKNLNSLSELEVKNLQRLSGAMDELHNEILELDEKVDDLRADTISSQ
IELAVLLSNEGIINSEDEHLLALERKLKKMLGPSAVDIGNGCFETKHKC
NQTCLDRIAAGTFNAGEFSLPTFDSLNITAASLNDDGLDNHTILLYYST
AASSLAVTLMLAIFIVGSAGLEVIFQGPHHHHHHHHHH*

The * denotes a stop codon in the sequences as provided in this disclosure.

SEQ ID NO: 9 depicts the nucleic acid sequence encoding a polypeptide expressed by a host cell transfected with sMH1_02HA0d-delHis

GACACTCTGTGCATCGGATACCACGCTAACAACTCCACCGACACTGTGG
ACACCGTCCTGGAGAAGAACGTCACCGTGACCCACTCTGTGAACCTGCT
GGAGGACAAGCACAACGGCAAGCTGTGCAAGCTGAGAGGCGTGGCCCC
ACTGCACCTGGGCAAGTGCAACATCGCTGGCTGGATACTGGGTAACCCT
GAGTGCGAAAGCCTGTCCACTGCTAGATCATGGTCCTACATCGTGGAAA
CTTCCAACAGCGACAACGGTACTTGCTACCCAGGAGACTTCATCAACTA
CGAGGAGCTGAGGGAGCAGCTGTCATCTGTGTCCAGCTTCGAAAGGTT
CGAAATCTTCCCAAAGACTTCATCTTGGCCTAACCACGACAGCGACAAG
GGTGTGACTGCTGCCTGCCCTCACGCTGGTGCCAAGTCATTCTACAAGA
ACCTGATCTGGCTGGTGAAGAAGGGCAACTCATACCCTAAGCTGAACC
AGACTTACATCAACGACAAGGGCAAGGAGGTCCTGGTGCTGTGGGGTA
TCCACCACCCTCCCACCATCGCTGCCCAGGAGTCTCTGTACCAGAACGC
TGACGCTTACGTGTTCGTCGGTACTTCACGCTACTCCAAGAAGTTCAAG
CCCGAAATCGCTACTCGCCCAAAGGTGCGCGACCAGGAAGGCAGGATG
AACTACTACTGGACTCTGGTGGAACCTGGAGACAAGATCACCTTCGAG
GCTACTGGAAACCTGGTCGTGCCAAGATACGCTTTCACTATGGAGCGTG
ACGCTGGTTCTGGCATCATCATCTCCGACACTCCTGTCCACGACTGCAA
CACCACTTGCCAGACCCCAGAGGGTGCTATCAACACCTCTCTGCCATTC
CAGAACGTGCACCCAATCACCATCGGAAAGTGCCCCAAGTACGTGAAG
TCTACCAAGCTGCGTCTGGCTACTGGACTGAGGAACGTGCCATCTATCC
AGTCACGCGGTCTGTTCGGTGCTATCGCTGGCTTCATCGAGGGCGGTTG
GACTGGCATGGTGGACGGTTGGTACGGATACCACCACCAGAACGAGCA
GGGCTCAGGTTACGCTGCTGACCTGAAGTCAACCCAGAACGCTATCGA
CAAGATCACTAACAAGGTGAACTCTGTGATCGAAAAGATGAACACCCA
GTTCACCGCTGTGGGAAAGGAGTTCAACCACCTGGAGAAGCGTATCGA
GAACCTGAACAAGAAGGTGGACGACGGATTCCTGGACATCTGGACCTA
CAACGCTGAGCTGCTGGTCCTGCTGGAAAACGAGCGCACCCTGGACTA
CCACGACAGCAACGTCAAGAACCTGTACGAGAAGGTGCGCAACCAGCT
GAAGAACAACGCTAAGGAGATCGGCAACGGCTGCTTCGAGTTCTACCA
CAAGTGCGACAACACTTGCATGGAATCCGTGAAGAACGGTACTTACGA
CTACCCAAAGTACTCTGAGGAGGCCAAGCTGAACAGGGAGAAGATCGA
CGGCGTGAAGCTGGAGTCCACTCGCATCTACCAGATCCTGGCTATCTAC
TCAACTGTGGCCTCTTCACTGGTGCTGGTCGTGTCTCTGGGTGCTATCA
GCTTCTGGATGGGTTCAGCTGGTTAA

SEQ ID NO: 10 depicts the amino acid sequence of a polypeptide expressed b the host cell transfected with sMH1_02HA0d-delHis

DTLCIGYHANNSTDTVDTVLEKNVTVTHSVNLLEDKHNGKLCKLRGVAP
LHLGKCNIAGWILGNPECESLSTARSWSYIVETSNSDNGTCYPGDFINY
EELREQLSSVSSFERFEIFPKTSSWPNHDSDKGVTAACPHAGAKSFYKN
LIWLVKKGNSYPKLNQTYINDKGKEVLVLWGIHHPPTIAAQESLYQNAD
AYVFVGTSRYSKKFKPEIATRPKVRDQEGRMNYYWTLVEPGDKITFEAT
GNLVVPRYAFTMERDAGSGIIISDTPVHDCNTTCQTPEGAINTSLPFQN
VHPITIGKCPKYVKSTKLRLATGLRNVPSIQSRGLFGAIAGFIEGGWTG
MVDGWYGYHHQNEQGSGYAADLKSTQNAIDKITNKVNSVIEKMNTQFTA
VGKEFNHLEKRIENLNKKVDDGFLDIWTYNAELLVLLENERTLDYHDSN
VKNLYEKVRNQLKNNAKEIGNGCFEFYHKCDNTCMESVKNGTYDYPKYS
EEAKLNREKIDGVKLESTRIYQILAIYSTVASSLVLVVSLGAISFWMGS
AG*

The * denotes a stop codon in the sequences as provided in this disclosure. SEQ ID NO: 11 depicts the nucleic acid sequence encoding a polypeptide expressed by a host cell transfected with sMH3_02HA0d-delHis

CAGAAGATCCCCGGCAACGACAACTCAACCGCTACTCTGTGCCTGGGA
CACCACGCCGTGCCAAACGGCACCATCGTCAAGACCATCACTAACGAC
CGTATCGAGGTGACCAACGCTACTGAACTGGTCCAGAACTCCAGCATC
GGAGAAATCTGCGACTCCCCACACCAGATCCTGGACGGTGGCAACTGC
ACTCTGATCGACGCTCTGCTGGGCGACCCTCAGTGCGACGGATTCCAGA
ACAAGAAGTGGGACCTGTTCGTGGAACGCTCTCGTGCCTACTCAAACTG
CTACCCTTACGACGTGCCCGACTACGCTTCTCTGAGGTCACTGGTCGCC
TCTTCAGGAACCCTGGAGTTCAAGAACGAATCTTTCAACTGGGCTGGTG
TCACTCAGAACGGCAAGTCCTTCAGCTGCATCCGTGGTTCCAGCTCTTC
ATTCTTCTCAAGGCTGAACTGGCTGACCCACCTGAACTACACTTACCCC
GCCCTGAACGTGACCATGCCAAACAAGGAGCAGTTCGACAAGCTGTAC
ATCTGGGGAGTCCACCACCCCGGTACTGACAAGGACCAGATCAGCCTG
TACGCTCAGTCCAGCGGTAGAATCACCGTGTCCACTAAGCGCAGCCAG
CAGGCCGTCATCCCCAACATCGGCAGCAGGCCAAGAATCCGCGACATC
CCTTCCAGAATCAGCATCTACTGGACCATCGTGAAGCCAGGCGACATCC
TGCTGATCAACTCCACTGGCAACCTGATCGCTCCTCGTGGATACTTCAA
GATCAGGTCCGGCAAGTCCTCCATCATGCGCAGCGACGCCCCTATCGGA
AAGTGCAAGTCTGAGTGCATCACCCCCAACGGTTCAATCCCAAACGAC
AAGCCTTTCCAGAACGTGAACCGTATCACTTACGGCGCTTGCCCTCGTT
ACGTCAAGCAGAACACCCTGAAGCTGGCCACTGGAATGAGAAACGTGC
CCGAAAAGCAGACCCGCGGTATCTTCGGCGCTATCGCCGGCTTCATCGA
GAACGGATGGGAGGGTATGGTGGACGGCTGGTACGGATTCCGTCACCA
GAACTCCGAGGGAAGGGGTCAGGCTGCCGACCTGAAGAGCACCCAGGC
TGCCATCGACCAGATCAACGGCAAGCTGAACAGACTGATCGGAAAGAC
TAACGAAAAGTTCCACCAGATCGAGAAGGAGTTCAGCGAGGTGGAAGG
TCGCGTCCAGGACCTGGAGAAGTACGTGGAGGACACCAAGATCGACCT
GTGGTCCTACAACGCTGAGCTGCTGGTCGCCCTGGAAAACCAGCACAC
CATCGACCTGACTGACAGCGAGATGAACAAGCTGTTCGAAAAGACCAA
GAAGCAGCTGCGTGAGAACGCTGAGGACATGGGCAACGGATGCTTCAA
GATTTACCACAAGTGCGACAACGCCTGCATCGGTTCTATCAGGAACGA
GACTTACGACCACAACGTGTACAGAGACGAAGCTCTGAACAACCGCTT
CCAGATCAAGGGTGTCGAGCTGAAGTCAGGCTACAAGGACTGGATCTT
GTGGATCTCTTTCGCCATCTCATGCTTCCTGCTGTGCGTGGCTCTGCTG
GGTTTCATCATGGGTTCAGCTGGTTAA

SEQ ID NO: 12 depicts the amino acid sequence of a polypeptide expressed by a host cell transfected with sMH3_02HA0d-delHis

QKIPGNDNSTATLCLGHHAVPNGTIVKTITNDRIEVTNATELVQNSSIG
EICDSPHQILDGGNCTLIDALLGDPQCDGFQNKKWDLFVERSRAYSNCY
PYDVPDYASLRSLVASSGTLEFKNESFNWAGVTQNGKSFSCIRGSSSSF
FSRLNWLTHLNYTYPALNVTMPNKEQFDKLYIWGVHHPGTDKDQISLYA
QSSGRITVSTKRSQQAVIPNIGSRPRIRDIPSRISIYWTIVKPGDILLI
NSTGNLIAPRGYFKIRSGKSSIMRSDAPIGKCKSECITPNGSIPNDKPF
QNVNRITYGACPRYVKQNTLKLATGMRNVPEKQTRGIFGAIAGFIENGW
EGMVDGWYGFRHQNSEGRGQAADLKSTQAAIDQINGKLNRLIGKTNEKF
HQIEKEFSEVEGRVQDLEKYVEDTKIDLWSYNAELLVALENQHTIDLTD
SEMNKLFEKTKKQLRENAEDMGNGCFKIYHKCDNACIGSIRNETYDHNV
YRDEALNNRFQIKGVELKSGYKDWILWISFAISCFLLCVALLGFIMGSA
G*

SEQ ID NO: 13 depicts the nucleic acid sequence encoding a polypeptide expressed by a host cell transfected with sMHV_02HA0d-delHis

GACAGGATCTGCACCGGTATCACTTCCAGCAACAGCCCCCACGTGGTCA
AGACTGCTACCCAGGGAGAAGTGAACGTCACTGGTGTGATCCCCCTGA
CCACTACCCCAACCAAGTCTCACTTCGCCAACCTGAAGGGCACTGAAAC
CCGCGGCAAGCTGTGCCCAAAGTGCCTGAACTGCACTGACCTGGACGT
GGCTCTGGGCAGACCCAAGTGCACCGGAAAGATCCCATCCGCCCGCGT
CAGCATCCTGCACGAAGTGCGTCCTGTCACTTCTGGCTGCTTCCCCATC
ATGCACGACCGCACCAAGATCCGTCAGCTGCCTAACCTGCTGAGGGGTT
ACGAGCACGTGAGACTGTCAACTCACAACGTCATCAACGCTGAGGACG
CCCCAGGCCGCCCTTACGAGATCGGCACCTCCGGCAGCTGCCCCAACAT
CACTAACGGAAACGGTTTCTTCGCTACTATGGCTTGGGCCGTCCCAAAG
AACAAGACTGCCACCAACCCTCTGACCATCGAAGTGCCCTACATCTGCA
CCGAGGGCGAGGACCAGATCACTGTCTGGGGATTCCACTCCGACAACG
AGACTCAGATGGCTAAGCTGTACGGCGACAGCAAGCCTCAGAAGTTCA
CCTCTTCAGCCAACGGAGTGACTACCCACTACGTCTCCCAGATCGGTGG
CTTCCCAAACCAGACCGAGGACGGAGGTCTGCCTCAGTCTGGTCGTATC
GTGGTGGACTACATGGTGCAGAAGTCAGGAAAGACTGGCACCATCACT
TACCAGCGCGGAATCCTGCTGCCTCAGAAAGTGTGGTGCGCTTCTGGTC
GTTCAAAGGTCATCAAGGGTTCCCTGCCCCTGATCGGTGAAGCTGACTG
CCTGCACGAGAAGTACGGCGGACTGAACAAGAGCAAGCCCTACTACAC
TGGAGAACACGCTAAGGCCATCGGTAACTGCCCAATCTGGGTCAAGAC
TCCTCTGAAGCTGGCTAACGGCACCAAGTACAGGCCTCCCGCCAAGCTG
CTGAAGGAGAGAGGCTTCTTCGGAGCTATCGCCGGTTTCCTGGAAGGTG
GCTGGGAGGGCATGATCGCTGGTTGGCACGGCTACACCTCCCACGGTG
CTCACGGTGTGGCTGTGGCTGCCGACCTGAAGTCAACTCAGGAAGCCAT
CAACAAGATCACCAAGAACCTGAACTCTCTGTCAGAGCTGGAAGTGAA
GAACCTGCAACGCCTGTCCGGAGCTATGGACGAGCTGCACAACGAGAT
CCTGGAACTGGACGAGAAGGTGGACGACCTGCGTGCTGACACCATCTC
CAGCCAGATCGAACTGGCCGTCCTGCTGTCCAACGAGGGAATCATCAA
CAGCGAGGACGAACACCTGCTGGCTCTGGAAAGGAAGCTGAAGAAGAT
GCTGGGTCCAAGCGCCGTGGAAATCGGCAACGGATGCTTCGAAACCAA
GCACAAGTGCAACCAGACTTGCCTGGACAGAATCGCTGCCGGTACTTTC
GACGCTGGCGAGTTCTCTCTGCCTACCTTCGACTCACTGAACATCACTG
CTGCCTCTCTGAACGACGACGGCCTGGACAACCACACCATCCTGCTGTA
CTACTCAACTGCTGCCTCTTCACTGGCTGTCACCCTGATGATCGCCATC
TTCGTGGTCGGTTCAGCTGGTTAA

SEQ ID NO: 14 depicts the amino acid sequence of a polypeptide expressed by the host cell transfected with sMHV_02HA0d-delHis

DRICTGITSSNSPHVVKTATQGEVNVTGVIPLTTTPTKSHFANLKGTET
RGKLCPKCLNCTDLDVALGRPKCTGKIPSARVSILHEVRPVTSGCFPIM
HDRTKIRQLPNLLRGYEHVRLSTHNVINAEDAPGRPYEIGTSGSCPNIT
NGNGFFATMAWAVPKNKTATNPLTIEVPYICTEGEDQITVWGFHSDNET
QMAKLYGDSKPQKFTSSANGVTTHYVSQIGGFPNQTEDGGLPQSGRIVV
DYMVQKSGKTGTITYQRGILLPQKVWCASGRSKVIKGSLPLIGEADCLH
EKYGGLNKSKPYYTGEHAKAIGNCPIWVKTPLKLANGTKYRPPAKLLKE
RGFFGAIAGFLEGGWEGMIAGWHGYTSHGAHGVAVAADLKSTQEAINKI
TKNLNSLSELEVKNLQRLSGAMDELHNEILELDEKVDDLRADTISSQIE
LAVLLSNEGIINSEDEHLLALERKLKKMLGPSAVEIGNGCFETKHKCNQ
TCLDRIAAGTFDAGEFSLPTFDSLNITAASLNDDGLDNHTILLYYSTAA
SSLAVTLMIAIFVVGSAG*

SEQ ID NO: 15 depicts the nucleic acid sequence of a polypeptide expressed b a host cell transfected with sMHY 01HA0d-delHis

GACAGGATCTGCACTGGTATCACCTCCAGCAACTCACCTCACGTGGTCA
AGACTGCTACCCAGGGTGAAGTGAACGTCACCGGCGTGATCCCCCTGA
CCACTACCCCAACTAAGTCCTACTTCGCCAACCTGAAGGGAACTAGGAC
CCGCGGCAAGCTGTGCCCTGACTGCCTGAACTGCACCGACCTGGACGTG
GCTCTGGGAAGGCCCATGTGCGTCGGTACTACCCCATCCGCTAAGGCCA
GCATCCTGCACGAGGTGCGCCCTGTCACCTCTGGCTGCTTCCCCATCAT
GCACGACAGGACTAAGATCAGACAGCTGCCAAACCTGCTGCGCGGATA
CGAAAAGATCCGTCTGTCAACTCAGAACGTCATCGACGCTGAGAAGGC
CCCAGGTGGCCCTTACCGCCTGGGCACTTCCGGAAGCTGCCCTAACGCT
ACCTCCAAGATCGGCTTCTTCGCCACTATGGCTTGGGCCGTGCCCAAGG
ACAACTACAAGAACGCCACTAACCCTCTGACCGTGGAAGTCCCCTACAT
CTGCACTGAGGGAGAGGACCAGATCACCGTCTGGGGTTTCCACAGCGA
CAACAAGACCCAGATGAAGTCTCTGTACGGAGACTCAAACCCTCAGAA
GTTCACTTCTTCAGCTAACGGTGTGACTACCCACTACGTCTCTCAGATC
GGCGACTTCCCAGACCAGACCGAGGACGGAGGTCTGCCTCAGTCAGGT
CGTATCGTGGTGGACTACATGATGCAGAAGCCTGGCAAGACCGGCACC
ATCGTGTACCAGCGCGGAGTCCTGCTGCCACAGAAAGTGTGGTGCGCTT
CTGGTCGTTCAAAGGTCATCAAGGGCTCCCTGCCACTGATCGGAGAAGC
CGACTGCCTGCACGAGGAATACGGCGGACTGAACAAGAGCAAGCCTTA
CTACACCGGCAAGCACGCTAAGGCCATCGGCAACTGCCCAATCTGGGT
CAAGACCCCTCTGAAGCTGGCTAACGGCACTAAGTACAGGCCTCCCGC
CAAGCTGCTGAAGGAGAGAGGATTCTTCGGTGCTATCGCTGGCTTCCTG
GAAGGTGGCTGGGAGGGAATGATCGCTGGCTGGCACGGATACACCTCC
CACGGTGCTCACGGCGTGGCTGTGGCTGCCGACCTGAAGAGCACCCAG
GAAGCCATCAACAAGATCACTAAGAACCTGAACTCTCTGTCAGAGCTG
GAAGTGAAGAACCTGCAACGCCTGAGCGGAGCTATGGACGAGCTGCAC
AACGAGATCCTGGAACTGGACGAGAAGGTGGACGACCTGCGTGCTGAC
ACCATCTCCAGCCAGATCGAACTGGCCGTCCTGCTGTCTAACGAGGGTA
TCATCAACTCAGAGGACGAACACCTGCTGGCTCTGGAAAGGAAGCTGA
AGAAGATGCTGGGTCCTTCCGCTGTGGACATCGGAAACGGTTGCTTCGA
GACTAAGCACAAGTGCAACCAGACCTGCCTGGACAGAATCGCTGCCGG
CACCTTCAACGCTGGAGAGTTCTCCCTGCCAACTTTCGACAGCCTGAAC
ATCACCGCTGCCTCCCTGAACGACGACGGTCTGGACAACCACACTATCC
TGCTGTACTACAGCACCGCTGCCTCTTCACTGGCTGTGACTCTGATGCT
GGCCATCTTCATCGTGGGTTCAGCTGGTTAA

SEQ ID NO: 16 depicts the amino acid sequence of a polypeptide expressed the host cell transfected with sMHY_04HA0d-delHis

DRICTGITSSNSPHVVKTATQGEVNVTGVIPLTTTPTKSYFANLKGTRT
RGKLCPDCLNCTDLDVALGRPMCVGTTPSAKASILHEVRPVTSGCFPIM
HDRTKIRQLPNLLRGYEKIRLSTQNVIDAEKAPGGPYRLGTSGSCPNAT
SKIGFFATMAWAVPKDNYKNATNPLTVEVPYICTEGEDQITVWGFHSDN
KTQMKSLYGDSNPQKFTSSANGVTTHYVSQIGDFPDQTEDGGLPQSGRI
VVDYMMQKPGKTGTIVYQRGVLLPQKVWCASGRSKVIKGSLPLIGEADC
LHEEYGGLNKSKPYYTGKHAKAIGNCPIWVKTPLKLANGTKYRPPAKLL
KERGFFGAIAGFLEGGWEGMIAGWHGYTSHGAHGVAVAADLKSTQEAIN
KITKNLNSLSELEVKNLQRLSGAMDELHNEILELDEKVDDLRADTISSQ
IELAVLLSNEGIINSEDEHLLALERKLKKMLGPSAVDIGNGCFETKHKC
NQTCLDRIAAGTFNAGEFSLPTFDSLNITAASLNDDGLDNHTILLYYST
AASSLAVTLMLAIFIVGSAG*

SEQ ID NO: 17 depicts the nucleic acid sequence encoding a polypeptide expressed by a host cell transfected with sMH1_04HA0d-Tg

GACACCCTGTGCATCGGATACCACGCCAACAACTCTACCGACACTGTGG
ACACTGTCCTGGAGAAGAACGTGACCGTCACTCACTCCGTGAACCTGCT
GGAAGACAAGCACAACGGCAAGCTGTGCAAGCTGAGGGGAGTCGCTCC
TCTGCACCTGGGCAAGTGCAACATCGCCGGTTGGATCTTGGGCAACCCC
GAGTGCGAATCCCTGAGCACCGCTCGCTCCTGGTCATACATCGTGGAGA
CCTCCAACAGCGACAACGGAACTTGCTACCCTGGTGACTTCATCAACTA
CGAGGAACTGAGGGAACAGCTGTCCAGCGTGTCTTCATTCGAGAGATT
CGAAATCTTCCCAAAGACCTCCAGCTGGCCTAACCACGACTCTGACAAC
GGTGTGACTGCTGCTTGCCCACACGCTGGAGCCAAGTCATTCTACAAGA
ACCTGATCTGGCTGGTCAAGAAGGGCAAGTCCTACCCAAAGATCAACC
AGACCTACATCAACGACAAGGGCAAGGAGGTGCTGGTCCTGTGGGGCA
TCCACCACCCTCCCACTATCGCTGACCAGCAGTCCCTGTACCAGAACGC
TGACGCCTACGTGTTCGTCGGAACCTCTCGCTACTCAAAGAAGTTCAAG
CCCGAGATCGCCACTCGCCCAAAGGTGCGTGACCAGGAAGGTCGTATG
AACTACTACTGGACCCTGGTCGAGCCCGGTGACAAGATCACCTTCGAA
GCTACTGGCAACCTGGTGGCTCCACGCTACGCCTTCACTATGGAGCGTG
ACGCCGGTTCCGGTATCATCATCAGCGACACTCCAGTCCACGACTGCAA
CACCACTTGCCAGACCCCTGAAGGTGCTATCAACACTTCACTGCCTTTC
CAGAACGTGCACCCCATCACCATCGGAAAGTGCCCAAAGTACGTCAAG
TCCACCAAGCTGAGGCTGGCCACTGGTCTGAGAAACGTGCCTTCCATCC
AGAGCCGTGGACTGTTCGGTGCTATCGCCGGATTCATCGAGGGTGGCTG
GACTGGTATGGTGGACGGCTGGTACGGATACCACCACCAGAACGAACA
GGGCAGCGGATACGCTGCCGACCTGAAGTCTACCCAGAACGCTATCGA
CAAGATCACTAACAAGGTGAACAGCGTCATCGAGAAGATGAACACCCA
GTTCACTGCCGTGGGCAAGGAGTTCAACCACCTGGAGAAGCGCATCGA
AAACCTGAACAAGAAGGTGGACGACGGATTCCTGGACATCTGGACCTA
CAACGCTGAGCTGCTGGTCCTGCTGGAGAACGAAAGGACTCTGGACTA
CCACGACTCTAACGTGAAGAACCTGTACGAAAAGGTCAGAAACCAGCT
GAAGAACAACGCCAAGGAGATCGGTAACGGCTGCTTCGAGTTCTACCA
CAAGTGCGACAACACCTGCATGGAGAGCGTGAAGAACGGTACTTACGA
CTACCCCAAGTACTCTGAGGAAGCTAAGCTGAACCGCGAAAAGATCGA
CGGCGTCAAGCTGGACTCAACCCGTATCTACCAGATCCTGGCTATCTAC
TCCACTGTGGCCTCTTCACTGGTCCTGGTGGTCTCTCTGGGCGCTATCT
CATTCTGGATGGGTTCCGCTAGCTAG

SEQ ID NO: 18 depicts the amino acid sequence of a polypeptide expressed by the host cell transfected with sMH1_04HA0d-Tg

DTLCIGYHANNSTDTVDTVLEKNVTVTHSVNLLEDKHNGKLCKLRGVAP
LHLGKCNIAGWILGNPECESLSTARSWSYIVETSNSDNGTCYPGDFINY
EELREQLSSVSSFERFEIFPKTSSWPNHDSDNGVTAACPHAGAKSFYKN
LIWLVKKGKSYPKINQTYINDKGKEVLVLWGIHHPPTIADQQSLYQNAD
AYVFVGTSRYSKKFKPEIATRPKVRDQEGRMNYYWTLVEPGDKITFEAT
GNLVAPRYAFTMERDAGSGIIISDTPVHDCNTTCQTPEGAINTSLPFQN
VHPITIGKCPKYVKSTKLRLATGLRNVPSIQSRGLFGAIAGFIEGGWTG
MVDGWYGYHHQNEQGSGYAADLKSTQNAIDKITNKVNSVIEKMNTQFTA
VGKEFNHLEKRIENLNKKVDDGFLDIWTYNAELLVLLENERTLDYHDSN
VKNLYEKVRNQLKNNAKEIGNGCFEFYHKCDNTCMESVKNGTYDYPKYS
EEAKLNREKIDGVKLDSTRIYQILAIYSTVASSLVLVVSLGAISFWMGS
AS*

SEQ ID NO: 19 depicts the nucleic acid sequence encoding a polypeptide expressed by a host cell transfected with sMH3_03HA0d-Tg

CAGAAGATCCCTGGTAACGACAACAGCACCGCTACTCTGTGCCTGGGA
CACCACGCCGTGCCCAACGGCACCATCGTCAAGACCATCACTAACGAC
CGTATCGAGGTGACCAACGCCACTGAACTGGTCCAGAACTCCAGCATC
GGCGAAATCTGCGACTCCCCTCACCAGATCCTGGACGGTGGCAACTGC
ACTCTGATCGACGCTCTGCTGGGCGACCCTCAGTGCGACGGATTCCAGA
ACAAGGAGTGGGACCTGTTCGTGGAACGCTCTCGTGCCAACTCAAACT
GCTACCCTTACGACGTGCCCGACTACGCTTCCCTGAGGAGCCTGGTCGC
CTCTTCAGGCACCCTGGAGTTCAAGAACGAATCCTTCAACTGGACCGGA
GTCAAGCAGAACGGTACTTCCAGCGCTTGCATCCGTGGCTCTTCATCCA
GCTTCTTCAGCAGGCTGAACTGGCTGACCCACCTGAACTACACTTACCC
CGCCCTGAACGTGACCATGCCAAACAACGAGCAGTTCGACAAGCTGTA
CATCTGGGGCGTCCACCACCCATCTACTGACAAGGACCAGATCAGCCTG
TTCGCTCAGCCTAGCGGCAGAATCACCGTGTCCACTAAGCGCAGCCAGC
AGGCCGTCATCCCCAACATCGGATCTAGGCCAAGAATCCGCGACATCC
CTTCCCGCATCTCCATCTACTGGACCATCGTGAAGCCCGGAGACATCCT
GCTGATCAACTCAACTGGCAACCTGATCGCTCCACGTGGATACTTCAAG
ATCAGGTCTGGCAAGTCCTCCATCATGCGCTCAGACGCCCCTATCGGCA
AGTGCAAGTCCGAGTGCATCACCCCCAACGGCAGCATCCCAAACGACA
AGCCTTTCCAGAACGTGAACCGTATCACTTACGGAGCTTGCCCACGTTA
CGTCAAGCAGAGCACCCTGAAGCTGGCCACTGGTATGAGAAACGTGCC
TGAAAAGCAGACCCGCGGTATCTTCGGCGCTATCGCCGGCTTCATCGAG
AACGGATGGGAGGGTATGGTGGACGGCTGGTACGGATTCCGTCACCAG
AACTCTGAGGGAAGGGGTCAGGCTGCCGACCTGAAGTCAACCCAGGCT
GCCATCGACCAGATCAACGGCAAGCTGAACAGACTGATCGGAAAGACT
AACGAAAAGTTCCACCAGATCGAGAAGGAGTTCTCCGAGGTGGAAGGT
CGCGTCCAGGACCTGGAGAAGTACGTGGAAGACACTAAGATCGACCTG
TGGTCTTACAACGCTGAGCTGCTGGTCGCCCTGGAAAACCAGCACACCA
TCGACCTGACTGACTCAGAGATGAACAAGCTGTTCGAAAAGACCAAGA
AGCAGCTGCGTGAGAACGCTGAAGACATGGGCAACGGATGCTTCAAGA
TTTACCACAAGTGCGACAACGCCTGCATCGGTTCCATCAGGAACGAGA
CTTACGACCACAACGTGTACAGAGACGAAGCTCTGAACAACCGCTTCC
AGATCAAGGGTGTCGAGCTGAAGAGCGGCTACAAGGACTGGATCTTGT
GGATCTCTTTCGCTATGTCATGCTTCCTGCTGTGCATCGCCCTGCTGGG
TTTCATCATGGGCTCTGCTAGCTAG

SEQ ID NO: 20 depicts the amino acid sequence of a polypeptide expressed by the host cell transfected with sMH3_03HA0d-Tg

QKIPGNDNSTATLCLGHHAVPNGTIVKTITNDRIEVTNATELVQNSSI
GEICDSPHQILDGGNCTLIDALLGDPQCDGFQNKEWDLFVERSRANSN
CYPYDVPDYASLRSLVASSGTLEFKNESFNWTGVKQNGTSSACIRGSS
SSFFSRLNWLTHLNYTYPALNVTMPNNEQFDKLYIWGVHHPSTDKDQI
SLFAQPSGRITVSTKRSQQAVIPNIGSRPRIRDIPSRISIYWTIVKPG
DILLINSTGNLIAPRGYFKIRSGKSSIMRSDAPIGKCKSECITPNGSI
PNDKPFQNVNRITYGACPRYVKQSTLKLATGMRNVPEKQTRGIFGAIA
GFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALE
NQHTIDLTDSEMNKLFEKTKKQLRENAEDMGNGCFKIYHKCDNACIGS
IRNETYDHNVYRDEALNNRFQIKGVELKSGYKDWILWISFAMSCFLLC
IALLGFIMGSAS*

SEQ ID NO: 21 depicts the nucleic acid sequence encoding a polypeptide expressed by a host cell transfected with sMH1_03HA0d

GACACCCTGTGCATCGGCTACCACGCTAACAACAGCACCGACACTGTG
GACACTGTCCTGGAGAAGAACGTGACCGTCACTCACAGCGTGAACCTG
CTGGAAGACAAGCACAACGGCAAGCTGTGCAAGCTGAGGGGAGTCGCT
CCTCTGCACCTGGGCAAGTGCAACATCGCCGGTTGGATCTTGGGCAACC
CCGAGTGCGAATCCCTGAGCACCGCTCGCTCCTGGTCATACATCGTGGA
AACCTCCAACAGCGACAACGGAACTTGCTACCCTGGTGACTTCATCAAC
TACGAGGAACTGAGGGAACAGCTGTCCAGCGTGTCTTCATTCGAGAGA
TTCGAAATCTTCCCAAAGACCTCCAGCTGGCCTAACCACGACTCTGACA
ACGGTGTGACTGCTGCTTGCCCACACGCTGGAGCCAAGTCTTTCTACAA
GAACCTGATCTGGCTGGTCAAGAAGGGCAAGTCATACCCAAAGATCAA
CCAGACCTACATCAACGACAAGGGCAAGGAGGTGCTGGTCCTGTGGGG
CATCCACCACCCTCCCACTATCGCTGACCAGCAGTCCCTGTACCAGAAC
GCTGACGCCTACGTGTTCGTCGGAACCTCCCGCTACAGCAAGAAGTTCA
AGCCCGAGATCGCCACTCGCCCAAAGGTGCGTGACCAGGAAGGTCGTA
TGAACTACTACTGGACCCTGGTCGAGCCCGGTGACAAGATCACCTTCGA
AGCTACTGGCAACCTGGTGGCTCCACGCTACGCCTTCACTATGGAGCGT
GACGCCGGTTCCGGTATCATCATCAGCGACACTCCAGTCCACGACTGCA
ACACCACTTGCCAGACCCCTGAAGGAGCTATCAACACTTCTCTGCCTTT
CCAGAACGTGCACCCCATCACCATCGGAAAGTGCCCAAAGTACGTCAA
GTCAACCAAGCTGAGGCTGGCCACTGGTCTGAGAAACGTGCCTTCTATC
CAGTCACGTGGACTGTTCGGTGCTATCGCCGGATTCATCGAGGGTGGCT
GGACTGGTATGGTGGACGGCTGGTACGGATACCACCACCAGAACGAAC
AGGGCTCCGGATACGCTGCCGACCTGAAGAGCACCCAGAACGCTATCG
ACAAGATCACTAACAAGGTGAACTCCGTCATCGAGAAGATGAACACCC
AGTTCACTGCCGTGGGCAAGGAGTTCAACCACCTGGAGAAGCGCATCG
AAAACCTGAACAAGAAGGTGGACGACGGATTCCTGGACATCTGGACCT
ACAACGCTGAGCTGCTGGTCCTGCTGGAGAACGAAAGGACTCTGGACT
ACCACGACAGCAACGTGAAGAACCTGTACGAAAAGGTCAGAAACCAGC
TGAAGAACAACGCCAAGGAGATCGGTAACGGCTGCTTCGAGTTCTACC
ACAAGTGCGACAACACCTGCATGGAGTCCGTGAAGAACGGTACTTACG
ACTACCCCAAGTACAGCGAGGAAGCTAAGCTGAACCGCGAAAAGATCG
ACGGCGTCAAGCTGGACTCTACCCGTATCTACCAGATCCTGGCTATCTA
CTCAACTGTGGCCTCTTCACTGGTCCTGGTGGTGTCCCTGGGCGCTATC
TCATTCTGGATGGGATCTGCTAGCCTGGAGGTTCTCTTCCAGGGTCCAG
GATCCCATCACCACCATCATCACCATCACCACCACTAA

SEQ ID NO: 22 depicts the amino acid sequence of a polypeptide expressed by the host cell transfected with sMH1_03HA0d

DTLCIGYHANNSTDTVDTVLEKNVTVTHSVNLLEDKHNGKLCKLRGVAP
LHLGKCNIAGWILGNPECESLSTARSWSYIVETSNSDNGTCYPGDFINY
EELREQLSSVSSFERFEIFPKTSSWPNHDSDNGVTAACPHAGAKSFYKN
LIWLVKKGKSYPKINQTYINDKGKEVLVLWGIHHPPTIADQQSLYQNAD
AYVFVGTSRYSKKFKPEIATRPKVRDQEGRMNYYWTLVEPGDKITFEAT
GNLVAPRYAFTMERDAGSGIIISDTPVHDCNTTCQTPEGAINTSLPFQN
VHPITIGKCPKYVKSTKLRLATGLRNVPSIQSRGLFGAIAGFIEGGWTG
MVDGWYGYHHQNEQGSGYAADLKSTQNAIDKITNKVNSVIEKMNTQFTA
VGKEFNHLEKRIENLNKKVDDGFLDIWTYNAELLVLLENERTLDYHDSN
VKNLYEKVRNQLKNNAKEIGNGCFEFYHKCDNTCMESVKNGTYDYPKYS
EEAKLNREKIDGVKLDSTRIYQILAIYSTVASSLVLVVSLGAISFWMGS
ASLEVLFQGPGSHHHHHHHHHH*

SEQ ID NO: 23 depicts the nucleic acid sequence encoding a polypeptide expressed by a host cell transfected with sMH3_S22_HA0d

CAGAAGATCCCCGGCAACGATAATTCGACCGCCACCCTGTGCCTGGGA
CACCACGCCGTGCCAAACGGCACCATCGTGAAGACCATCACCAACGAC
CGCATCGAGGTGACCAATGCCACCGAGCTGGTGCAGAACAGCTCCATC
GGCGAGATTTGCGGCAGCCCACACCAGATCCTGGATGGCGGAAATTGC
ACCCTGATTGATGCCCTGCTGGGCGATCCCCAGTGCGACGGCTTCCAGA
ACAAGGAGTGGGATCTGTTCGTGGAGCGCTCGCGCGCCAACAGCAATT
GCTACCCCTACGATGTGCCCGACTACGCCTCCCTGCGCTCGCTGGTGGC
CAGCAGCGGCACCCTGGAGTTCAAGAACGAGTCCTTCAATTGGACCGG
CGTGAAGCAGAATGGCACCTCCTCGGCCTGCATCCGCGGCAGCAGCAG
CAGCTTCTTCAGCCGCCTGAACTGGCTGACCAGCCTGAACAATATCTAC
CCGGCCCAGAACGTGACCATGCCAAATAAGGAGCAGTTCGACAAGCTG
TACATCTGGGGCGTGCACCACCCGGATACCGACAAGAATCAGATCAGC
CTGTTCGCCCAGTCCTCGGGCCGCATCACCGTGTCCACCAAGCGCTCGC
AGCAGGCCGTGATCCCCAACATTGGCAGCCGCCCACGCATCCGCGACA
TCCCCAGCCGCATCTCCATCTACTGGACCATCGTGAAGCCAGGCGACAT
CCTGCTGATCAACAGCACCGGCAATCTGATCGCCCCACGCGGCTACTTC
AAGATCCGCTCCGGCAAGAGCAGCATCATGCGCTCGGATGCCCCCATC
GGCAAGTGCAAGAGCGAGTGCATCACCCCGAACGGCTCCATCCCAAAT
GACAAGCCCTTCCAGAACGTGAATCGCATTACCTACGGCGCCTGCCCAC
GCTACGTGAAGCAGTCCACCCTGAAGCTGGCCACCGGAATGCGCAATG
TGCCCGAGAAGCAGACCCGCGGCATCTTCGGAGCCATTGCCGGCTTCAT
CGAGAATGGCTGGGAGGGCATGGTGGATGGCTGGTACGGCTTCCGCCA
CCAGAACTCGGAGGGACGCGGACAGGCCGCCGATCTGAAGAGCACCCA
GGCCGCCATCGACCAGATCAACGGCAAGCTGAATCGCCTGATCGGCAA
GACCAACGAGAAGTTCCACCAGATCGAGAAGGAGTTCTCGGAGGTGGA
GGGACGCGTGCAGGACCTGGAGAAGTACGTGGAGGATACCAAGATCGA
CCTGTGGAGCTACAATGCCGAGCTGCTGGTGGCCCTGGAGAACCAGCA
CACCATCGATCTGACCGACTCCGAGATGAATAAGCTGTTCGAGAAGAC
CAAGAAGCAGCTGCGCGAGAACGCCGAGGATATGGGCAATGGCTGCTT
CAAGATTTACCACAAGTGCGACAACGCCTGCATCGGCTCCATCCGCAAC
GAGACGTACGATCACAATGTGTACCGCGACGAGGCCCTGAACAATCGC
TTCCAGATCAAGGGCGTGGAGCTGAAGTCGGGCTACAAGGATTGGATT
CTGTGGATCAGCTTCGCCATGTCCTGCTTCCTGCTGTGCATCGCCCTGCT
GGGCTTCATCATGGGCTCGGCTAGCCTGGAGGTGCTGTTTCAGGGACCA
GGATCCCATCATCATCATCATCATCATCATCATCACTAA

SEQ ID NO: 24 depicts the amino acid sequence of a polypeptide expressed by the host cell transfected with sMH3_S22_HA0d

QKIPGNDNSTATLCLGHHAVPNGTIVKTITNDRIEVTNATELVQNSSI
GEICGSPHQILDGGNCTLIDALLGDPQCDGFQNKEWDLFVERSRANSN
CYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQI
SLFAQSSGRITVSTKRSQQAVIPNIGSRPRIRDIPSRISIYWTIVKPG
DILLINSTGNLIAPRGYFKIRSGKSSIMRSDAPIGKCKSECITPNGSI
PNDKPFQNVNRITYGACPRYVKQSTLKLATGMRNVPEKQTRGIFGAIA
GFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALE
NQHTIDLTDSEMNKLFEKTKKQLRENAEDMGNGCFKIYHKCDNACIGS
IRNETYDHNVYRDEALNNRFQIKGVELKSGYKDWILWISFAMSCFLLC
IALLGFIMGSASLEVLFQGPGSHHHHHHHHHH*

SEQ ID NO: 25 depicts the nucleic acid sequence encoding a polypeptide expressed by a host cell transfected with sMH3_S22_HA0d-Tg

CAGAAGATCCCCGGCAACGATAATTCGACCGCCACCCTGTGCCTGGGA
CACCACGCCGTGCCAAACGGCACCATCGTGAAGACCATCACCAACGAC
CGCATCGAGGTGACCAATGCCACCGAGCTGGTGCAGAACAGCTCCATC
GGCGAGATTTGCGGCAGCCCACACCAGATCCTGGATGGCGGAAATTGC
ACCCTGATTGATGCCCTGCTGGGCGATCCCCAGTGCGACGGCTTCCAGA
ACAAGGAGTGGGATCTGTTCGTGGAGCGCTCGCGCGCCAACAGCAATT
GCTACCCCTACGATGTGCCCGACTACGCCTCCCTGCGCTCGCTGGTGGC
CAGCAGCGGCACCCTGGAGTTCAAGAACGAGTCCTTCAATTGGACCGG
CGTGAAGCAGAATGGCACCTCCTCGGCCTGCATCCGCGGCAGCAGCAG
CAGCTTCTTCAGCCGCCTGAACTGGCTGACCAGCCTGAACAATATCTAC
CCGGCCCAGAACGTGACCATGCCAAATAAGGAGCAGTTCGACAAGCTG
TACATCTGGGGCGTGCACCACCCGGATACCGACAAGAATCAGATCAGC
CTGTTCGCCCAGTCCTCGGGCCGCATCACCGTGTCCACCAAGCGCTCGC
AGCAGGCCGTGATCCCCAACATTGGCAGCCGCCCACGCATCCGCGACA
TCCCCAGCCGCATCTCCATCTACTGGACCATCGTGAAGCCAGGCGACAT
CCTGCTGATCAACAGCACCGGCAATCTGATCGCCCCACGCGGCTACTTC
AAGATCCGCTCCGGCAAGAGCAGCATCATGCGCTCGGATGCCCCCATC
GGCAAGTGCAAGAGCGAGTGCATCACCCCGAACGGCTCCATCCCAAAT
GACAAGCCCTTCCAGAACGTGAATCGCATTACCTACGGCGCCTGCCCAC
GCTACGTGAAGCAGTCCACCCTGAAGCTGGCCACCGGAATGCGCAATG
TGCCCGAGAAGCAGACCCGCGGCATCTTCGGAGCCATTGCCGGCTTCAT
CGAGAATGGCTGGGAGGGCATGGTGGATGGCTGGTACGGCTTCCGCCA
CCAGAACTCGGAGGGACGCGGACAGGCCGCCGATCTGAAGAGCACCCA
GGCCGCCATCGACCAGATCAACGGCAAGCTGAATCGCCTGATCGGCAA
GACCAACGAGAAGTTCCACCAGATCGAGAAGGAGTTCTCGGAGGTGGA
GGGACGCGTGCAGGACCTGGAGAAGTACGTGGAGGATACCAAGATCGA
CCTGTGGAGCTACAATGCCGAGCTGCTGGTGGCCCTGGAGAACCAGCA
CACCATCGATCTGACCGACTCCGAGATGAATAAGCTGTTCGAGAAGAC
CAAGAAGCAGCTGCGCGAGAACGCCGAGGATATGGGCAATGGCTGCTT
CAAGATTTACCACAAGTGCGACAACGCCTGCATCGGCTCCATCCGCAAC
GAGACGTACGATCACAATGTGTACCGCGACGAGGCCCTGAACAATCGC
TTCCAGATCAAGGGCGTGGAGCTGAAGTCGGGCTACAAGGATTGGATT
CTGTGGATCAGCTTCGCCATGTCCTGCTTCCTGCTGTGCATCGCCCTGCT
GGGCTTCATCATGGGCTCGGCTAGCTAA

SEQ ID NO: 26 depicts the amino acid sequence of a polypeptide expressed by a host cell transfected with sMH3_S22_HA0d-Tg

QKIPGNDNSTATLCLGHHAVPNGTIVKTITNDRIEVTNATELVQNSSI
GEICGSPHQILDGGNCTLIDALLGDPQCDGFQNKEWDLFVERSRANSN
CYPYDVPDYASLRSLVASSGTLEFKNESFNWTGVKQNGTSSACIRGSS
SSFFSRLNWLTSLNNIYPAQNVTMPNKEQFDKLYIWGVHHPDTDKNQI
SLFAQSSGRITVSTKRSQQAVIPNIGSRPRIRDIPSRISIYWTIVKPG
DILLINSTGNLIAPRGYFKIRSGKSSIMRSDAPIGKCKSECITPNGSI
PNDKPFQNVNRITYGACPRYVKQSTLKLATGMRNVPEKQTRGIFGAIA
GFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALE
NQHTIDLTDSEMNKLFEKTKKQLRENAEDMGNGCFKIYHKCDNACIGS
IRNETYDHNVYRDEALNNRFQIKGVELKSGYKDWILWISFAMSCFLLC
IALLGFIMGSAS*

SEQ ID NO: 27 depicts the nucleic acid sequence encoding a polypeptide expressed by the host cell transfected with sMHV_S22_HA0d

GATCGCATCTGCACCGGCATCACCAGCTCCAACTCGCCGCACGTGGTGA
AGACCGCCACCCAGGGCGAAGTGAATGTGACCGGCGTGATCCCCCTGA
CCACCACCCCAACCAAGAGCCACTTCGCCAACCTGAAGGGCACCGAGA
CGCGCGGCAAGCTGTGCCCAAAGTGCCTGAATTGCACCGATCTGGACG
TGGCCCTGGGCCGCCCAAAGTGCACCGGCAAGATCCCATCGGCCCGCG
TGTCGATTCTGCACGAGGTGCGCCCCGTGACCTCCGGCTGCTTCCCGAT
CATGCACGATCGCACCAAGATTCGCCAGCTGCCAAATCTGCTGCGCGG
ATACGAGCACGTGCGCCTGAGCACCCACAACGTGATCAATGCCGAGGA
TGCCCCAGGCCGCCCCTACGAGATCGGCACCTCGGGCAGCTGCCCGAA
TATCACCAACGGCAATGGCTTCTTCGCCACGATGGCCTGGGCCGTGCCA
AAGAACAAGACCGCCACCAATCCCCTGACCATCGAGGTGCCGTACATC
TGCACCGAGGGCGAGGATCAGATCACCGTGTGGGGCTTCCACAGCGAC
AACGAGACGCAGATGGCCAAGCTGTACGGCGATTCCAAGCCCCAGAAG
TTCACCAGCAGCGCCAACGGCGTGACCACCCACTACGTGTCCCAGATCG
GCGGATTCCCAAATCAGACCGAGGATGGCGGCCTGCCACAGTCCGGCC
GCATCGTGGTGGACTACATGGTGCAGAAGTCGGGCAAGACCGGCACCA
TCACCTACCAGCGCGGCATCCTGCTGCCGCAGAAAGTGTGGTGCGCCTC
GGGCCGCAGCAAGGTCATCAAGGGCTCGCTGCCCCTGATTGGAGAGGC
CGACTGCCTGCACGAGAAGTACGGCGGCCTGAACAAGAGCAAGCCGTA
CTACACCGGCGAGCACGCCAAGGCCATCGGCAACTGCCCAATCTGGGT
GAAGACCCCGCTGAAGCTGGCCAATGGCACCAAGTACCGCCCACCCGC
CAAGCTGCTGAAGGAGCGCGGCTTCTTCGGAGCCATTGCCGGCTTCCTG
GAGGGCGGCTGGGAGGGAATGATTGCCGGCTGGCACGGCTACACCTCG
CACGGCGCCCACGGCGTGGCCGTGGCCGCCGATCTGAAGAGCACCCAG
GAGGCCATCAATAAGATCACCAAGAACCTGAACAGCCTGTCGGAGCTG
GAGGTGAAGAACCTGCAGCGCCTGAGCGGCGCTATGGATGAGCTGCAC
AATGAGATCCTGGAGCTGGATGAGAAGGTGGATGACCTGCGCGCCGAC
ACCATCTCCTCGCAGATCGAGCTGGCCGTGCTGCTGTCGAACGAGGGCA
TCATCAATTCGGAGGATGAGCACCTGCTGGCCCTGGAGCGCAAGCTGA
AGAAGATGCTGGGACCATCGGCCGTGGAGATTGGAAACGGCTGCTTCG
AGACGAAGCACAAGTGCAATCAGACCTGCCTGGATCGCATTGCCGCCG
GCACCTTCGATGCCGGAGAGTTCAGCCTGCCCACCTTCGATTCCCTGAA
CATCACCGCCGCCAGCCTGAATGATGATGGCCTGGACAATCACACCATC
CTGCTGTACTACTCGACAGCCGCCAGCTCCCTGGCCGTGACCCTGATGA
TCGCCATCTTCGTGGTGGGCAGCGCTAGCCTGGAGGTGCTGTTCCAGGG
ACCAGGATCCCATCATCATCATCATCATCATCATCATCACTAA

SEQ ID NO: 28 depicts the amino acid sequence of a polypeptide expressed by the host cell transfected with sMHV_S22_HA0d

DRICTGITSSNSPHVVKTATQGEVNVTGVIPLTTTPTKSHFANLKGT
ETRGKLCPKCLNCTDLDVALGRPKCTGKIPSARVSILHEVRPVTSGC
FPIMHDRTKIRQLPNLLRGYEHVRLSTHNVINAEDAPGRPYEIGTSG
SCPNITNGNGFFATMAWAVPKNKTATNPLTIEVPYICTEGEDQITVW
GFHSDNETQMAKLYGDSKPQKFTSSANGVTTHYVSQIGGFPNQTEDG
GLPQSGRIVVDYMVQKSGKTGTITYQRGILLPQKVWCASGRSKVIKG
SLPLIGEADCLHEKYGGLNKSKPYYTGEHAKAIGNCPIWVKTPLKLA
NGTKYRPPAKLLKERGFFGAIAGFLEGGWEGMIAGWHGYTSHGAHGV
AVAADLKSTQEAINKITKNLNSLSELEVKNLQRLSGAMDELHNEILE
LDEKVDDLRADTISSQIELAVLLSNEGIINSEDEHLLALERKLKKML
GPSAVEIGNGCFETKHKCNQTCLDRIAAGTFDAGEFSLPTFDSLNIT
AASLNDDGLDNHTILLYYSTAASSLAVTLMIAIFVVGSASLEVLFQG
PGSHHHHHHHHHH*

SEQ ID NO: 29 depicts the nucleic acid sequence encoding a polypeptide expressed by a host cell transfected with sMHV_S22_HA0d-Tg

GATCGCATCTGCACCGGCATCACCAGCTCCAACTCGCCGCACGTGGTGA
AGACCGCCACCCAGGGCGAAGTGAATGTGACCGGCGTGATCCCCCTGA
CCACCACCCCAACCAAGAGCCACTTCGCCAACCTGAAGGGCACCGAGA
CGCGCGGCAAGCTGTGCCCAAAGTGCCTGAATTGCACCGATCTGGACG
TGGCCCTGGGCCGCCCAAAGTGCACCGGCAAGATCCCATCGGCCCGCG
TGTCGATTCTGCACGAGGTGCGCCCCGTGACCTCCGGCTGCTTCCCGAT
CATGCACGATCGCACCAAGATTCGCCAGCTGCCAAATCTGCTGCGCGG
ATACGAGCACGTGCGCCTGAGCACCCACAACGTGATCAATGCCGAGGA
TGCCCCAGGCCGCCCCTACGAGATCGGCACCTCGGGCAGCTGCCCGAA
TATCACCAACGGCAATGGCTTCTTCGCCACGATGGCCTGGGCCGTGCCA
AAGAACAAGACCGCCACCAATCCCCTGACCATCGAGGTGCCGTACATC
TGCACCGAGGGCGAGGATCAGATCACCGTGTGGGGCTTCCACAGCGAC
AACGAGACGCAGATGGCCAAGCTGTACGGCGATTCCAAGCCCCAGAAG
TTCACCAGCAGCGCCAACGGCGTGACCACCCACTACGTGTCCCAGATCG
GCGGATTCCCAAATCAGACCGAGGATGGCGGCCTGCCACAGTCCGGCC
GCATCGTGGTGGACTACATGGTGCAGAAGTCGGGCAAGACCGGCACCA
TCACCTACCAGCGCGGCATCCTGCTGCCGCAGAAAGTGTGGTGCGCCTC
GGGCCGCAGCAAGGTCATCAAGGGCTCGCTGCCCCTGATTGGAGAGGC
CGACTGCCTGCACGAGAAGTACGGCGGCCTGAACAAGAGCAAGCCGTA
CTACACCGGCGAGCACGCCAAGGCCATCGGCAACTGCCCAATCTGGGT
GAAGACCCCGCTGAAGCTGGCCAATGGCACCAAGTACCGCCCACCCGC
CAAGCTGCTGAAGGAGCGCGGCTTCTTCGGAGCCATTGCCGGCTTCCTG
GAGGGCGGCTGGGAGGGAATGATTGCCGGCTGGCACGGCTACACCTCG
CACGGCGCCCACGGCGTGGCCGTGGCCGCCGATCTGAAGAGCACCCAG
GAGGCCATCAATAAGATCACCAAGAACCTGAACAGCCTGTCGGAGCTG
GAGGTGAAGAACCTGCAGCGCCTGAGCGGCGCTATGGATGAGCTGCAC
AATGAGATCCTGGAGCTGGATGAGAAGGTGGATGACCTGCGCGCCGAC
ACCATCTCCTCGCAGATCGAGCTGGCCGTGCTGCTGTCGAACGAGGGCA
TCATCAATTCGGAGGATGAGCACCTGCTGGCCCTGGAGCGCAAGCTGA
AGAAGATGCTGGGACCATCGGCCGTGGAGATTGGAAACGGCTGCTTCG
AGACGAAGCACAAGTGCAATCAGACCTGCCTGGATCGCATTGCCGCCG
GCACCTTCGATGCCGGAGAGTTCAGCCTGCCCACCTTCGATTCCCTGAA
CATCACCGCCGCCAGCCTGAATGATGATGGCCTGGACAATCACACCATC
CTGCTGTACTACTCGACAGCCGCCAGCTCCCTGGCCGTGACCCTGATGA
TCGCCATCTTCGTGGTGGGCAGCGCTAGCTAA

SEQ ID NO: 30 depicts the amino acid sequence of a polypeptide expressed by a host cell transfected with sMHV_S22_HA0d-Tg

DRICTGITSSNSPHVVKTATQGEVNVTGVIPLTTTPTKSHFANLKGTE
TRGKLCPKCLNCTDLDVALGRPKCTGKIPSARVSILHEVRPVTSGCFP
IMHDRTKIRQLPNLLRGYEHVRLSTHNVINAEDAPGRPYEIGTSGSCP
NITNGNGFFATMAWAVPKNKTATNPLTIEVPYICTEGEDQITVWGFHS
DNETQMAKLYGDSKPQKFTSSANGVTTHYVSQIGGFPNQTEDGGLPQS
GRIVVDYMVQKSGKTGTITYQRGILLPQKVWCASGRSKVIKGSLPLIG
EADCLHEKYGGLNKSKPYYTGEHAKAIGNCPIWVKTPLKLANGTKYRP
PAKLLKERGFFGAIAGFLEGGWEGMIAGWHGYTSHGAHGVAVAADLKS
TQEAINKITKNLNSLSELEVKNLQRLSGAMDELHNEILELDEKVDDLR
ADTISSQIELAVLLSNEGIINSEDEHLLALERKLKKMLGPSAVEIGNG
CFETKHKCNQTCLDRIAAGTFDAGEFSLPTFDSLNITAASLNDDGLDN
HTILLYYSTAASSLAVTLMIAIFVVGSAS*

SEQ ID NO: 31 depicts the amino acid sequence of a full-length HA protein having cytoplasmic domain (wild type antigen), obtained from A/Hawaii/70/2019 (H1N1)pdm09-like virus strain.

MKAILVVLLYTFTTANADTLCIGYHANNSTDTVDTVLEKNVTVTHSVN
LLEDKHNGKLCKLRGVAPLHLGKCNIAGWILGNPECESLSTARSWSYI
VETSNSDNGTCYPGDFINYEELREQLSSVSSFERFEIFPKTSSWPNHD
SDKGVTAACPHAGAKSFYKNLIWLVKKGNSYPKLNQTYINDKGKEVLV
LWGIHHPPTIAAQESLYQNADAYVFVGTSRYSKKFKPEIATRPKVRDQ
EGRMNYYWTLVEPGDKITFEATGNLVVPRYAFTMERDAGSGIIISDTP
VHDCNTTCQTPEGAINTSLPFQNVHPITIGKCPKYVKSTKLRLATGLR
NVPSIQSRGLFGAIAGFIEGGWTGMVDGWYGYHHQNEQGSGYAADLKS
TQNAIDKITNKVNSVIEKMNTQFTAVGKEFNHLEKRIENLNKKVDDGF
LDIWTYNAELLVLLENERTLDYHDSNVKNLYEKVRNQLKNNAKEIGNG
CFEFYHKCDNTCMESVKNGTYDYPKYSEEAKLNREKIDGVKLESTRIY
QILAIYSTVASSLVLVVSLGAISFWMCSNGSLQCRICI

SEQ ID NO: 32 depicts the nucleic acid sequence encoding for the signal peptide

ATGCTCCTCGTCAACCAGTCACATCAAGGCTTCAACAAAGAACATACCT
CAAAAATGGTTTCCGCTATCGTCCTCTACGTGCTGCTGGCTGCTGCTGC
CCACTCTGCTTTCGCT

SEQ ID NO: 33 depicts the nucleic acid sequence of a recombinant construct sMH1_02HA0d comprising nucleic acid encoding for a signal peptide, HRV3C recognition sequence, and histidine tag sequence

GAATTCGCCGCCACCATGCTCCTCGTCAACCAGTCACATCAAGG
CTTCAACAAAGAACATACCTCAAAAATGGTTTCCGCTATCGTCCTCTAC
GTGCTGCTGGCTGCTGCTGCCCACTCTGCTTTCGCTGACACTCTGTGCAT
CGGATACCACGCTAACAACTCCACCGACACTGTGGACACCGTCCTGGA
GAAGAACGTCACCGTGACCCACTCTGTGAACCTGCTGGAGGACAAGCA
CAACGGCAAGCTGTGCAAGCTGAGAGGCGTGGCCCCACTGCACCTGGG
CAAGTGCAACATCGCTGGCTGGATACTGGGTAACCCTGAGTGCGAAAG
CCTGTCCACTGCTAGATCATGGTCCTACATCGTGGAAACTTCCAACAGC
GACAACGGTACTTGCTACCCAGGAGACTTCATCAACTACGAGGAGCTG
AGGGAGCAGCTGTCATCTGTGTCCAGCTTCGAAAGGTTCGAAATCTTCC
CAAAGACTTCATCTTGGCCTAACCACGACAGCGACAAGGGTGTGACTG
CTGCCTGCCCTCACGCTGGTGCCAAGTCATTCTACAAGAACCTGATCTG
GCTGGTGAAGAAGGGCAACTCATACCCTAAGCTGAACCAGACTTACAT
CAACGACAAGGGCAAGGAGGTCCTGGTGCTGTGGGGTATCCACCACCC
TCCCACCATCGCTGCCCAGGAGTCTCTGTACCAGAACGCTGACGCTTAC
GTGTTCGTCGGTACTTCACGCTACTCCAAGAAGTTCAAGCCCGAAATCG
CTACTCGCCCAAAGGTGCGCGACCAGGAAGGCAGGATGAACTACTACT
GGACTCTGGTGGAACCTGGAGACAAGATCACCTTCGAGGCTACTGGAA
ACCTGGTCGTGCCAAGATACGCTTTCACTATGGAGCGTGACGCTGGTTC
TGGCATCATCATCTCCGACACTCCTGTCCACGACTGCAACACCACTTGC
CAGACCCCAGAGGGTGCTATCAACACCTCTCTGCCATTCCAGAACGTGC
ACCCAATCACCATCGGAAAGTGCCCCAAGTACGTGAAGTCTACCAAGC
TGCGTCTGGCTACTGGACTGAGGAACGTGCCATCTATCCAGTCACGCGG
TCTGTTCGGTGCTATCGCTGGCTTCATCGAGGGCGGTTGGACTGGCATG
GTGGACGGTTGGTACGGATACCACCACCAGAACGAGCAGGGCTCAGGT
TACGCTGCTGACCTGAAGTCAACCCAGAACGCTATCGACAAGATCACT
AACAAGGTGAACTCTGTGATCGAAAAGATGAACACCCAGTTCACCGCT
GTGGGAAAGGAGTTCAACCACCTGGAGAAGCGTATCGAGAACCTGAAC
AAGAAGGTGGACGACGGATTCCTGGACATCTGGACCTACAACGCTGAG
CTGCTGGTCCTGCTGGAAAACGAGCGCACCCTGGACTACCACGACAGC
AACGTCAAGAACCTGTACGAGAAGGTGCGCAACCAGCTGAAGAACAAC
GCTAAGGAGATCGGCAACGGCTGCTTCGAGTTCTACCACAAGTGCGAC
AACACTTGCATGGAATCCGTGAAGAACGGTACTTACGACTACCCAAAG
TACTCTGAGGAGGCCAAGCTGAACAGGGAGAAGATCGACGGCGTGAAG
CTGGAGTCCACTCGCATCTACCAGATCCTGGCTATCTACTCAACTGTGG
CCTCTTCACTGGTGCTGGTCGTGTCTCTGGGTGCTATCAGCTTCTGGATG
GGTTCAGCTGGTCTCGAAGTTTTGTTCCAGGGACCCCATCACCACCATC
ATCACCACCACCATCACTAAAAGCTT

SEQ ID NO: 34 depicts the nucleic acid sequence of HRV3C recognition sequence

CTCGAAGTTTTGTTCCAGGGACCC

SEQ ID NO: 35 depicts the amino acid sequence of HRV3C recognition sequence

LEVLFQGP

SEQ ID NO: 36 depicts the nucleic acid sequence of His tag

CATCACCACCATCATCACCACCACCATCAC

SEQ ID NO: 37 depicts the amino acid sequence of His tag

HHHHHHHHHH

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “polypeptide fragment” refers to the recombinant HA protein of influenza virus strains that is modified by deleting and replacing the cytoplasmic domain with a linker. The HA protein sequences are obtained from different strains of influenza virus (as per the recommendations of World Health Organisation (WHO)) or any future or past seasonal influenza vaccine strains recommended by WHO.

The term “immunogenic composition” refers to a composition comprising the polypeptide fragment along with adjuvant and other excipients that elicits a prophylactic or therapeutic immune response in a subject. In the present disclosure, the “immunogenic composition” and “vaccine” are used interchangeably.

Typically, a vaccine elicits an antigen-specific immune response to an antigen of a pathogen, for example a viral pathogen, or to a cellular constituent correlated with a pathological condition.

The term “vaccine candidate” refers to a polypeptide fragment that can be potentially used in a vaccine composition.

The term “subject” refers to any animal classified as a mammal, e.g., human and non-human mammals. Examples of non-human animals include non-human primates, dogs, cats, cattle, horses, sheep, pigs, goats, rabbits, mice, rats, hamsters, guinea pigs and etc. Unless otherwise noted, the terms “patient” or “subject” are used herein interchangeably. Preferably, the subject is human.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “excipient” refers to the component that is very known to a person skilled in the art that can be added as a vehicle alongside the polypeptide fragment in the immunogenic composition.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

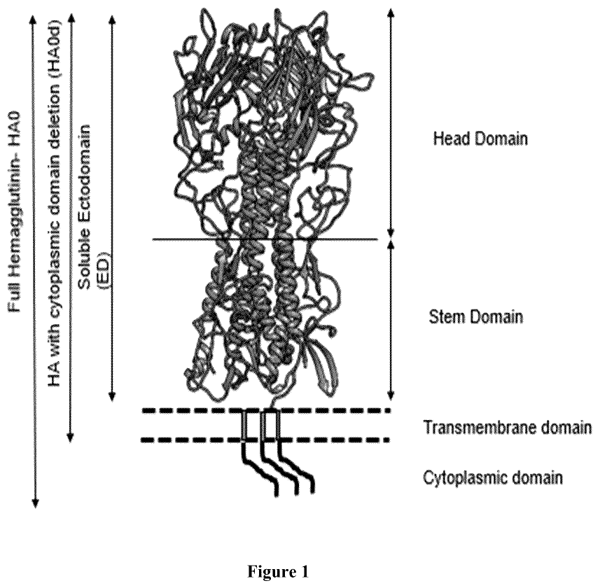

As discussed in the background section of the present disclosure, several strategies have been explored to develop effective vaccines against influenza viruses. These include the induction of antibodies directed against hemagglutinin (HA). Neutralizing antibodies (nAbs) against hemagglutinin (HA) are the primary correlate for protection in humans and hence HA is an attractive target for vaccine development (Gerhard W (2001) The role of the antibody response in influenza virus infection. Curr Top Microbiol Immunol 260:171-190). The precursor polypeptide, HA0, is assembled into a trimer along the secretory pathway and transported to the cell surface. Cleavage of HA0 generates the disulfide-linked HA1 and HA2 subunits. Mature HA has a globular head domain which mediates receptor binding and is primarily composed of the HA1 subunit, whereas the stem domain predominantly comprises the HA2 subunit. The HA stem is trapped in a metastable state and undergoes an extensive low-pH-induced conformational rearrangement in the host-cell endosomes to adopt the virus-host membrane fusion competent state (Carr C M, Kim P S (1993) A spring-loaded mechanism for the conformational change of influenza hemagglutinin. Cell 73(4):823-832; Skehel J J, Wiley DC (2000) Receptor binding and membrane fusion in virus entry: The influenza hemagglutinin. Annu Rev Biochem 69:531-569). FIG. 1 depicts the pictorial representation of domain organization of HA protein having head domain, stem domain, transmembrane domain, and cytoplasmic domain.

In the recent time, the use of recombinant vaccines has garnered attention of the masses. For instance, one such licensed vaccine is FluBlok, which is a recombinant vaccine with HA proteins that are expressed in insect cells from baculovirus vectors. Another HA based recombinant vaccine is a nanoparticle influenza vaccine (NIV) from Novavax, which is a novel seasonal recombinant HA nanoparticle influenza vaccine formulated with a saponin-based adjuvant, Matrix-M™.

Although currently available vaccines are effective against seasonal influenza viruses, strain-specific immunity fails to protect against drifted seasonal influenza viruses or from antigenically new pandemic viruses. Other limitations of the conventional vaccines include the dependence on embryonated eggs for vaccine production, the lengthy timeline for vaccine production, the need for annual vaccination, and the need for an improved correlate of protection. Further, the current HA-based recombinant influenza vaccines comprise a full-length sequence of the HA protein (wild type antigen) having the cytoplasmic domain, that induces only limited immune responses against different strains of influenza virus.

To overcome the aforementioned challenges and improve the immunogenicity and efficacy of influenza vaccines, the present disclosure provides a recombinant immunogenic composition containing modified hemagglutinin (HA) protein fragment of different influenza strains (as per the recommendations of World Health Organisation (WHO)) or any future or past seasonal influenza vaccine strains recommended by WHO, with a deletion of cytoplasmic domain (hereafter referred as (HA0d) or head design 3. The cytoplasmic domain of HA protein is deleted and replaced with a linker polypeptide, such as GSAG or GSSAG or similar linkers. The present disclosure thus solves the problem of potency loss associated with the existing vaccine compositions that are based on full length of HA protein sequence with cytoplasmic domain having cysteine residues. In the present disclosure, either a complete or a small portion (having a length in the range of 7 to 17 amino acid residues) of the cytoplasmic domain of HA gene of different influenza virus strains is deleted and replaced with the linker (GSAG or GSSAG), to arrive at modified polypeptide fragments that can be deployed as immunogenic composition against different circulating influenza virus strains (as recommended by WHO) or any future or past seasonal influenza vaccine strains recommended by WHO that may arise from time to time. The polypeptide fragment as described herein is in a form of a trimer or higher order aggregates, which is due to the presence of a transmembrane domain. In another embodiments, the present disclosure provides a recombinant immunogenic composition or fragment having modified hemagglutinin (HA) protein fragment of different influenza strains (as per the recommendations of World Health Organisation (WHO)) or any future or past seasonal influenza vaccine strains recommended by WHO with a deletion of the cytoplasmic domain (hereafter referred to as HA0d or head design 3). The deleted fragment is replaced with a linker peptide preferably of 2 to 5 amino acids in length, such as GSAG or GSSAG or similar linkers. An advantage of the deletion of cytoplasmic domain is that the cytoplasmic tail domain contains several Cys residues which can potentially result in non-specific disulphide bonded aggregates.

The polypeptide fragment is a recombinant polypeptide fragment comprising Hemagglutinin (HA) protein sequence derived from different strains of influenza virus that is recommended by WHO, wherein the HA protein sequence is modified by deleting and replacing the cytoplasmic domain with a linker. The different strains of influenza virus includes but not limited to A/Hawaii/70/2019, A/Hong Kong/45/2019, B/Washington/02/2019, B/Phuket/3073/2013, A/Wisconsin/588/2019, A/Cambodia/e0826360/2020, A/Darwin/6/2021, B/Austria/1359417/2021, A/California/7/2009, A/Hong Kong/4801/2014, B/Brisbane/60/2008, B/Phuket/3073/2013, A/Michigan/45/2015, A/Singapore/INFIMH-16-0019/2016, A/Switzerland/8060/2017, B/Colorado/06/2017, B/Phuket/3073/2013, A/Brisbane/02/2018, A/South Australia/34/2019, A/Kansas/14/2017, or any or past seasonal influenza vaccine strains recommended by WHO. The DNA and protein sequences are available for these strains from GISAID. All HA genes coding for the protein fragment (obtained from different influenza strains) were codon optimized for high-level expression in recombinant insect cells.

In an embodiment, there is provided a polypeptide fragment comprising a polypeptide of a modified Hemagglutinin (HA) protein, wherein said protein comprises a linker peptide replacing the polypeptide fragment in the cytoplasmic domain of a HA protein of influenza virus, wherein the HA protein is obtained from at least one or a plurality of strains of influenza virus. In another embodiment, there is provided a polypeptide fragment comprising a polypeptide of a modified Hemagglutinin (HA) protein, wherein said protein comprises a linker peptide replacing the polypeptide fragment of the cytoplasmic domain of the HA protein of influenza virus, wherein the hemagglutinin protein is obtained from at least one or a plurality of strains of influenza virus. The polypeptide fragment, in various embodiments herein, comprises the polypeptide of a modified HA protein. In an embodiment, the cytoplasmic domain of the HA protein obtained from one or more of the strains of influenza virus is deleted and replaced with a linker polypeptide. The linker polypeptide is a linker having a sequence as described herein (for example GSAG or GSSAG).

The polypeptide fragment comprises a polypeptide having at least 80% identity, or at least 90%, or 95-99% identity to a polypeptide having an amino acid selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30. In another embodiment of the present disclosure, the polypeptide fragment has an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

The present disclosure also discloses recombinant construct which is obtained by deleting the cytoplasmic domain of the HA gene sequence of the influenza strain and replacing the cytoplasmic domain with a linker (GSAG or GSSAG). Another embodiment provides a recombinant construct having hemagglutinin (HA) gene sequences of different influenza strains (as per the recommendations of World Health Organisation (WHO)) or any future or past seasonal influenza vaccine strains recommended by WHO with a deletion of the DNA sequences coding for cytoplasmic domain (HA0d) and addition of 2 to 5 or 1 to 3 amino acids. The deleted fragment is replaced with a DNA sequence coding for a linker, such as GSAG or GSSAG or similar linkers to obtain modified HA sequences. The modified HA gene sequences used for constructing the recombinant construct were codon optimized for expression in insect cells. The recombinant construct thus obtained comprises a nucleic acid fragment encoding the polypeptide fragments as described herein, operably linked to a promoter, and a nucleic acid sequence encoding a signal peptide (SEQ ID NO: 32). The recombinant construct optionally comprises a HRV3C recognition sequence (SEQ ID NO: 34), and a histidine tag sequence (SEQ ID NO: 36). The recombinant vector comprising the recombinant construct is also disclosed herein. The recombinant vector or recombinant construct as described herein is further used for transfecting a host cell (insect cells) to obtain a recombinant host cell. The recombinant host cell is further cultured under suitable conditions to obtain the polypeptide fragments as described herein, and the polypeptide fragment was then purified and contacted with an adjuvant (such as a squalene-in-water emulsion adjuvant) to obtain an immunogenic composition. The purification of the polypeptide fragment involves the removal of His tags. The purified proteins derived by cleaving a His tag sequence (amino acid sequence as set forth in SEQ ID NO: 37), has a protein sequence ending with either: (i) a linker, such as GSAG (as in the case of the protein sequences having an amino acid sequence as set forth in SEQ ID NO: 10, SEQ ID NO: 12, SEQ ID NO: 14, SEQ ID NO: 16, SEQ ID NO: 18, SEQ ID NO: 20, SEQ ID NO: 26, and SEQ ID NO: 30); or (ii) HRV3C recognition sequence (SEQ ID NO: 35), downstream of a linker (GSAG) present in SEQ ID NO: 10, SEQ ID NO: 12, SEQ ID NO: 14, SEQ ID NO: 16, SEQ ID NO: 18, SEQ ID NO: 20, SEQ ID NO: 26, and SEQ ID NO: 30. It can be concluded that these purified protein sequence ending with either a linker (GSAG), or HRV3C recognition sequence downstream of the linker, both can be used in the immunogenic composition.

The immunogenic composition is a multivalent immunogenic composition that elicits an enhanced immune response against different strains of influenza virus, which is represented by a significantly higher ELISA titers, Hemagglutinin Inhibition (HI) titers, and virus-neutralizing (VN) titers, as compared to that of the conventional vaccines. Although the immunogenic composition of the present disclosure elicits enhanced immune response against four different strains (H1N1, H3N2, Flu B Yamagata and Flu B Victoria) of influenza virus from which HA protein is obtained and modified to delete and replace the cytoplasmic domain with the linker. It can be contemplated that a person skilled in the art can delete the cytoplasmic domain of HA protein sequence obtain from different strains of influenza (as per the recommended WHO strains) or any future or past seasonal influenza vaccine strains recommended by WHO and can replace the cytoplasmic domain with a linker to arrive different polypeptide fragments that can be used for making the immunogenic composition against multiple influenza strains that may arise from time to time. The present disclosure discloses an immunogenic composition comprises a combination of four polypeptide fragments, said polypeptide fragment comprising HA protein sequence derived from different strains of influenza virus that is recommended by WHO, wherein the HA protein sequence is modified deleting and replacing the cytoplasmic domain with a linker, and wherein different strains of influenza virus is selected from the group consisting of:
(a) A/Hawaii/70/2019, A/Hong Kong/45/2019, B/Washington/02/2019, and B/Phuket/3073/2013; or
(b) A/Wisconsin/588/2019, A/Cambodia/e0826360/2020, B/Washington/02/2019, and B/Phuket/3073/2013; or
(c) A/Wisconsin/588/2019, A/Darwin/6/2021, B/Austria/1359417/2021, and B/Phuket/3073/2013; or
(d) A/California/7/2009, A/Hong Kong/4801/2014, B/Brisbane/60/2008, and B/Phuket/3073/2013; o(e) A/Michigan/45/2015, A/Hong Kong/4801/2014, B/Brisbane/60/2008, and B/Phuket/3073/2013; or
(f) A/Michigan/45/2015, A/Singapore/INFIMH-16-0019/2016, B/Colorado/06/2017, and B/Phuket/3073/2013; or
(g) A/Brisbane/02/2018, A/Kansas/14/2017, B/Colorado/06/2017, and B/Phuket/3073/2013; or
(h) A/Michigan/45/2015, A/Singapore/INFIMH-16-0019/2016, B/Brisbane/60/2008, and B/Phuket/3073/2013; or
(i) A/Michigan/45/2015, A/Switzerland/8060/2017, B/Colorado/06/2017, B/Phuket/3073/2013; or
(j) A/Brisbane/02/2018, A/South Australia/34/2019, B/Washington/02/2019, and B/Phuket/3073/2013; or
(k) A/Wisconsin/588/2019, A/Hong Kong/45/2019, B/Washington/02/2019, and B/Phuket/3073/2013; or any future or past seasonal influenza vaccine strains recommended by WHO.

The present disclosure in particular, provides an immunogenic composition comprising a combination of four polypeptide fragments having an amino acid sequence as set forth in SEQ ID NO: 10, SEQ ID NO: 12, SEQ ID NO: 14, and SEQ ID NO: 16, that is derived from HA protein sequences of four influenza virus strains—A/Hawaii/70/2019, A/Hong Kong/45/2019, B/Washington/02/2019, and B/Phuket/3073/2013 (as per WHO recommended strains for 2020-2021 influenza season). In another example, the present disclosure provides an immunogenic composition comprising a combination of four polypeptide fragments having an amino acid sequence as set forth in SEQ ID NO: 14, SEQ ID NO: 16, SEQ ID NO: 18, and SEQ ID NO: 20, that is derived from HA protein sequences of four influenza virus strains—B/Washington/02/2019, B/Phuket/3073/2013, A/Wisconsin/588/2019, A/Cambodia/e0826360/2020 (as per WHO recommended strains for 2021-2022 influenza season).

The immunogenic composition also provide protection against drifted strains that may arise due to mutations in influenza. Thus, the present disclosure provides an immunogenic composition that exhibits excellent stability, as well as enhanced immune responses against influenza viruses.

In an embodiment of the present disclosure, there is provided a polypeptide fragment comprising a polypeptide sequence of HA with cytoplasmic domain being deleted and replaced with a linker such as GSAG or GSSAG. Thus, the polypeptide fragment as described herein is a recombinant polypeptide fragment. The linker that is used for replacing the cytoplasmic domain of HA protein sequence can be linker having a length in the range of 2 to 9 amino acid residues, preferably 2 to 7 amino acid residues, most preferably 2 to 5 amino acid residues, or a linker that is known to a person skilled in the art.

In an embodiment of the present disclosure, there is provided a polypeptide fragment comprising a polypeptide having at least 80%, or at least 90%, or at least 95%, or at least 97% sequence identity to at least one amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30. In another embodiment of the present disclosure, the identity is 80-99%, or 81-98%, or 82-97%, or 83-96%.

In an embodiment of the present disclosure, there is provided a polypeptide fragment as described herein, wherein the polypeptide has at least 90%, at least 95%, or at least 97% sequence identity to at least one amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30. In another embodiment of the present disclosure, the identity is 91-99%, or 92-99%, or 93-98%, or 94-99%, or 95-99%. In yet another embodiment of the present disclosure, the identity is 91%, or 92%, or 93%, or 94%, or 95%, or 96%, or 97%, or 98%, or 99%. In an embodiment of the present disclosure, there is provided a polypeptide fragment as described herein, wherein the polypeptide has an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an embodiment of the present disclosure, there is provided a polypeptide fragment as described herein, wherein the polypeptide has an amino acid sequence selected from the group consisting of SEQ ID NO: 2, SEQ ID NO: 4, SEQ ID NO: 6, SEQ ID NO: 8, SEQ ID NO: 22, SEQ ID NO: 24, and SEQ ID NO: 28.

The polypeptide fragments comprising the polypeptide having an amino acid sequence selected from the group consisting of SEQ ID NO: 2, SEQ ID NO: 4, SEQ ID NO: 6, SEQ ID NO: 8, SEQ ID NO: 22, SEQ ID NO: 24, and SEQ ID NO: 28, comprises a linker, HRV3C recognition sequence, and a His tag sequence. The linker is GSAG, and the HRV3C recognition sequence has an amino acid sequence as set forth in SEQ ID NO: 35, and a His tag sequence has an amino acid sequence as set forth in SEQ ID NO: 37.

In an embodiment of the present disclosure, there is provided a polypeptide fragment as described herein, wherein the polypeptide has an amino acid sequence selected from the group consisting of SEQ ID NO: 10, SEQ ID NO: 12, SEQ ID NO: 14, SEQ ID NO: 16, SEQ ID NO: 18, SEQ ID NO: 20, SEQ ID NO: 26, and SEQ ID NO: 30.

In an embodiment of the present disclosure, there is provided a polypeptide fragment as described herein, wherein the polypeptide has an amino acid sequence selected from the group consisting of SEQ ID NO: 10, SEQ ID NO: 12, SEQ ID NO: 14, SEQ ID NO: 16, SEQ ID NO: 18, SEQ ID NO: 20, SEQ ID NO: 26, and SEQ ID NO: 30 and further comprises a HRV3C recognition sequence having an amino acid sequence as set forth in SEQ ID NO: 35, wherein the polypeptide is derived by cleaving the His tag, and wherein HRV3C recognition sequence is present downstream of a linker.

In an embodiment of the present disclosure, there is provided a nucleic acid fragment encoding the polypeptide fragment described herein. The nucleic acid fragment, according to embodiments herein, may be DNA, RNA or mRNA fragment. In an embodiment, the nucleic acid fragment is a DNA fragment. In another embodiment, the nucleic acid fragment is an RNA fragment. In yet another embodiment, the nucleic acid fragment is an mRNA fragment.

In an embodiment of the present disclosure, there is a nucleic acid fragment encoding the polypeptide fragment having at least 80%, or at least 90%, or at least 95%, or at least 97% sequence identity to a polypeptide having an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In another embodiment of the present disclosure, there is a nucleic acid fragment encoding the polypeptide fragment having at least 90%, or at least 95%, or at least 97%, or at least 99% sequence identity to a polypeptide having an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an embodiment of the present disclosure, there is a nucleic acid fragment encoding the polypeptide fragment having an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an embodiment of the present disclosure, there is provided a nucleic acid fragment as described herein, wherein the nucleic fragment having a nucleic acid sequence as set forth in SEQ ID NO: 1 encodes the polypeptide fragment having an amino acid sequence as set forth in SEQ ID NO: 2.

In an embodiment of the present disclosure, there is provided a nucleic acid fragment as described herein, wherein the nucleic fragment having a nucleic acid sequence as set forth in SEQ ID NO: 3 encodes the polypeptide fragment having an amino acid sequence as set forth in SEQ ID NO: 4.

In an embodiment of the present disclosure, there is provided a nucleic acid fragment as described herein, wherein the nucleic fragment having a nucleic acid sequence as set forth in SEQ ID NO: 5 encodes the polypeptide fragment having an amino acid sequence as set forth in SEQ ID NO: 6.

In an embodiment of the present disclosure, there is provided a nucleic acid fragment as described herein, wherein the nucleic fragment having a nucleic acid sequence as set forth in SEQ ID NO: 7 encodes the polypeptide fragment having an amino acid sequence as set forth in SEQ ID NO: 8.

In an embodiment of the present disclosure, there is provided a nucleic acid fragment as described herein, wherein the nucleic fragment having a nucleic acid sequence as set forth in SEQ ID NO: 21 encodes the polypeptide fragment having an amino acid sequence as set forth in SEQ ID NO: 22.

In an embodiment of the present disclosure, there is provided a nucleic acid fragment as described herein, wherein the nucleic fragment having a nucleic acid sequence as set forth in SEQ ID NO: 23 encodes the polypeptide fragment having an amino acid sequence as set forth in SEQ ID NO: 24.

In an embodiment of the present disclosure, there is provided a nucleic acid fragment as described herein, wherein the nucleic fragment having a nucleic acid sequence as set forth in SEQ ID NO: 27 encodes the polypeptide fragment having an amino acid sequence as set forth in SEQ ID NO: 28.

In an embodiment of the present disclosure, there is provided a nucleic acid fragment as described herein, wherein the nucleic fragment having a nucleic acid sequence as set forth in SEQ ID NO: 9 encodes the polypeptide fragment having an amino acid sequence as set forth in SEQ ID NO: 10.

In an embodiment of the present disclosure, there is provided a nucleic acid fragment as described herein, wherein the nucleic fragment having a nucleic acid sequence as set forth in SEQ ID NO: 11 encodes the polypeptide fragment having an amino acid sequence as set forth in SEQ ID NO: 12.

In an embodiment of the present disclosure, there is provided a nucleic acid fragment as described herein, wherein the nucleic fragment having a nucleic acid sequence as set forth in SEQ ID NO: 13 encodes the polypeptide fragment having an amino acid sequence as set forth in SEQ ID NO: 14.

In an embodiment of the present disclosure, there is provided a nucleic acid fragment as described herein, wherein the nucleic fragment having a nucleic acid sequence as set forth in SEQ ID NO: 15 encodes the polypeptide fragment having an amino acid sequence as set forth in SEQ ID NO: 16.

In an embodiment of the present disclosure, there is provided a nucleic acid fragment as described herein, wherein the nucleic fragment having a nucleic acid sequence as set forth in SEQ ID NO: 17 encodes the polypeptide fragment having an amino acid sequence as set forth in SEQ ID NO: 18.

In an embodiment of the present disclosure, there is provided a nucleic acid fragment as described herein, wherein the nucleic fragment having a nucleic acid sequence as set forth in SEQ ID NO: 19 encodes the polypeptide fragment having an amino acid sequence as set forth in SEQ ID NO: 20.

In an embodiment of the present disclosure, there is provided a nucleic acid fragment as described herein, wherein the nucleic fragment having a nucleic acid sequence as set forth in SEQ ID NO: 25 encodes the polypeptide fragment having an amino acid sequence as set forth in SEQ ID NO: 26.

In an embodiment of the present disclosure, there is provided a nucleic acid fragment as described herein, wherein the nucleic fragment having a nucleic acid sequence as set forth in SEQ ID NO: 29 encodes the polypeptide fragment having an amino acid sequence as set forth in SEQ ID NO: 30.

In an embodiment of the present disclosure, there is provided a recombinant construct comprising a nucleic acid fragment encoding a polypeptide fragment, operably linked to a promoter, said polypeptide fragment comprises a polypeptide having at least 80%, or at least 90%, or at least 95%, or at least 97% sequence identity to a polypeptide having an amino acid selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an embodiment of the present disclosure, there is provided a recombinant construct comprising a nucleic acid fragment encoding a polypeptide fragment, operably linked to a promoter, said polypeptide fragment comprises a polypeptide having at least 90% identity to a polypeptide having an amino acid selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an embodiment of the present disclosure, there is provided a recombinant construct comprising a nucleic acid fragment encoding a polypeptide fragment, operably linked to a promoter, said polypeptide fragment comprises a polypeptide having an amino acid selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

In an embodiment of the present disclosure, there is provided a recombinant construct as described herein, wherein the nucleic fragment has a nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, and SEQ ID NO: 29.

In an embodiment of the present disclosure, there is provided a recombinant construct as described herein, wherein the recombinant construct further comprises a nucleic acid sequence encoding a signal peptide, said nucleic acid sequence is as set forth in SEQ ID NO: 32. A person skilled in the art can use any known signal peptide sequence for the purpose of the present disclosure.

In an embodiment of the present disclosure, there is provided a recombinant construct as described herein, wherein the recombinant construct optionally comprises: (a) HRV3C recognition sequence; and (b) histidine tag sequence.

In an embodiment of the present disclosure, there is provided a recombinant construct comprising: (a) a nucleic acid fragment encoding a polypeptide fragment, said nucleic acid fragment has a nucleic acid sequence selected from the group consisting of SEQ ID NO: 1, SEQ ID NO: 3, SEQ ID NO: 5, SEQ ID NO: 7, SEQ ID NO: 21, SEQ ID NO: 23, and SEQ ID NO: 29; (b) a nucleic acid sequence encoding a signal peptide, the nucleic acid sequence is as set forth in SEQ ID NO: 32; and (c) HRV3C recognition sequence; and (d) histidine tag sequence.

In an embodiment of the present disclosure, there is provided a recombinant construct as described herein, wherein the HRV3C recognition sequence has a nucleic acid sequence as set forth in SEQ ID NO: 34, and an amino acid sequence as set forth in SEQ ID NO: 35.

In an embodiment of the present disclosure, there is provided a recombinant construct as described herein, wherein the His tag has a nucleic acid sequence as set forth in SEQ ID NO: 36, and an amino acid sequence as set forth in SEQ ID NO: 37.

In an embodiment of the present disclosure, there is provided a recombinant construct comprising: (a) a nucleic acid fragment encoding a polypeptide fragment; (b) a nucleic acid sequence encoding a signal peptide; and (c) HRV3C recognition sequence; and (d) histidine tag sequence, the recombinant construct has a nucleic acid sequence as set forth in SEQ ID NO: 33.

In an embodiment of the present disclosure, there is provided a recombinant vector comprising the recombinant construct as described herein.

In an embodiment of the present disclosure, there is provided a recombinant vector as described herein, wherein the recombinant vector is selected from the group consisting of pFastBac1, pDEST10, pDEST 20, and pIEx-2.

In an embodiment of the present disclosure, there is provided a recombinant host cell comprising the recombinant construct as described herein or the recombinant vector as described herein.

In an embodiment of the present disclosure, there is provided a recombinant host cell as described herein, wherein the recombinant host cell is selected from the group consisting of insect cell, bacterial cell, yeast cell, and mammalian cells. In another embodiment of the present disclosure, the recombinant host cell is an insect cell.

In an embodiment of the present disclosure, there is provided a recombinant host cell as described herein, wherein the insect cell is selected from the group consisting of Expi-Sj9®, Sf9, High Five®, Sf21, and S2, and wherein the bacterial cell is Escherichia coli, and wherein the yeast cell is selected from the group consisting of Pichia X33, Pichia GlycoSwitch®, DSMZ 70382, GS115, KM71, KM71H, BG09, GS190, GS200, JC220, JC254, JC227, JC300-JC308, YJN165, and CBS7435, and, wherein the mammalian cell is selected from the group consisting of Expi293F® Expi-CHO-S®, CHO-K1, CHO-S, HEK293F®, CHO^{BC}™, SLIM™, SPOT™, SP2/0, Sp2/0-Ag14, CHO DG44, HEK 293S, HEK 293 Gnt1^{-/-}, HEK293-EBNA1, CHOL-NSO, and NSO. In an embodiment of the present disclosure, there is provided an immunogenic composition comprising a combination of four polypeptide fragment, said polypeptide fragment comprising modified hemagglutinin protein, wherein said modified hemagglutinin protein comprises a linker peptide replacing the polypeptide fragment in the cytoplasmic domain of hemagglutinin protein obtained from at least one or a plurality of strains of influenza virus, and wherein the strains of influenza virus are selected from the group consisting of: immunogenic composition comprising a combination of four polypeptide fragments, said polypeptide fragment comprising HA protein sequence derived from different strains of influenza virus that is recommended by WHO, wherein the HA protein sequence is modified by deleting and replacing the cytoplasmic domain with a linker, and wherein different strains of influenza virus is selected from the group consisting of:
(a) A/Hawaii/70/2019, A/Hong Kong/45/2019, B/Washington/02/2019, and B/Phuket/3073/2013; or
(b) A/Wisconsin/588/2019, A/Cambodia/e0826360/2020, B/Washington/02/2019, and B/Phuket/3073/2013; or
(c) A/Wisconsin/588/2019, A/Darwin/6/2021, B/Austria/1359417/2021, and B/Phuket/3073/2013; or
(d) A/California/7/2009, A/Hong Kong/4801/2014, B/Brisbane/60/2008, and B/Phuket/3073/2013; or
(e) A/Michigan/45/2015, A/Hong Kong/4801/2014, B/Brisbane/60/2008, and B/Phuket/3073/2013; or
(f) A/Michigan/45/2015, A/Singapore/INFIMH-16-0019/2016, B/Colorado/06/2017, and B/Phuket/3073/2013; or
(g) A/Brisbane/02/2018, A/Kansas/14/2017, B/Colorado/06/2017, and B/Phuket/3073/2013; or
(h) A/Michigan/45/2015, A/Singapore/INFIMH-16-0019/2016, B/Brisbane/60/2008, and B/Phuket/3073/2013; or
(i) A/Michigan/45/2015, A/Switzerland/8060/2017, B/Colorado/06/2017, B/Phuket/3073/2013; or
(j) A/Brisbane/02/2018, A/South Australia/34/2019, B/Washington/02/2019, and B/Phuket/3073/2013; or
(k) A/Wisconsin/588/2019, A/Hong Kong/45/2019, B/Washington/02/2019, and B/Phuket/3073/2013.

In an embodiment of the present disclosure, there is provided an immunogenic composition comprising a combination of four polypeptide fragments having at least 80%, or at least 90%, or at least 95%, or at least 97% sequence identity to a polypeptide having an amino acid sequence as set forth in SEQ ID NO: 10, SEQ ID NO: 12, SEQ ID NO: 14, SEQ ID NO: 16, a pharmaceutically acceptable carrier. In another embodiment of the present disclosure, the identity is 80-99%, or 81-98%, or 82-97%, or 83-96%.

In an embodiment of the present disclosure, there is provided an immunogenic composition as described herein, wherein the immunogenic composition comprises a combination of four polypeptide fragments having at least 90% identity to a polypeptide having an amino acid sequence as set forth in SEQ ID NO: 10, SEQ ID NO: 12, SEQ ID NO: 14, SEQ ID NO: 16, a pharmaceutically acceptable carrier. In another embodiment of the present disclosure, the identity is 91-99%, or 92-99%, or 93-98%, or 94-99%, or 95-99%.

In an embodiment of the present disclosure, there is provided an immunogenic composition comprising a combination of four polypeptide fragments having an amino acid as SEQ ID NO: 10, SEQ ID NO: 12, SEQ ID NO: 14, SEQ ID NO: 16, and a pharmaceutically acceptable carrier.

In an embodiment of the present disclosure, there is provided an immunogenic composition comprising a combination of four polypeptide fragments having at least 80%, or at least 90%, or at least 95%, or at least 97% sequence identity to a polypeptide having an amino acid sequence as set forth in SEQ ID NO: 14, SEQ ID NO: 16, SEQ ID NO: 18, and SEQ ID NO: 20, a pharmaceutically acceptable carrier. In another embodiment of the present disclosure, the identity is 80-99%, or 81-98%, or 82-97%, or 83-96%.

In an embodiment of the present disclosure, there is provided an immunogenic composition comprising a combination of four polypeptide fragments having at least 90% identity to a polypeptide having an amino acid sequence as set forth in SEQ ID NO: 14, SEQ ID NO: 16, SEQ ID NO: 18, and SEQ ID NO: 20, a pharmaceutically acceptable carrier.

In an embodiment of the present disclosure, there is provided an immunogenic composition comprising a combination of four polypeptide fragments having an amino acid as SEQ ID NO: 14, SEQ ID NO: 16, SEQ ID NO: 18, and SEQ ID NO: 20, a pharmaceutically acceptable carrier.

In another embodiment of the present disclosure, there is provides an immunogenic composition as described herein, wherein the polypeptide fragment further comprises a HRV3C recognition sequence having an amino acid sequence as set forth in SEQ ID NO: 35, wherein the polypeptide is derived by cleaving a His tag, and wherein HRV3C recognition sequence is present downstream of a linker.

In an embodiment of the present disclosure, there is provided an immunogenic composition as described herein, wherein the immunogenic composition elicits immune response against influenza virus.

In an embodiment of the present disclosure, there is provided an immunogenic composition as described herein, wherein the immunogenic composition elicits immune response against influenza viral strains selected from the group consisting of H1N1, H3N2, Influenza B Yamagata and Influenza B Victoria.

In an embodiment of the present disclosure, there is provided an immunogenic composition as described herein, wherein the pharmaceutically acceptable carrier is selected from the group consisting of at least one adjuvant, and excipients. In another embodiment of the present disclosure, the pharmaceutically acceptable carrier is an adjuvant selected from the group consisting of squalene-in-water emulsion (SWE) adjuvant and chemically equivalent adjuvant.

In an embodiment of the present disclosure, there is provided an immunogenic composition as described herein, wherein the immunogenic composition is administered by a mode selected from the group consisting of intranasal, parenteral, subcutaneous, intravenous, intra-arterial, intra-peritoneal, intramuscular, intradermal, oral, dermal, nasal, and inhalation. In another embodiment of the present disclosure, the immunogenic composition is administered by intramuscular method.

In an embodiment of the present disclosure, there is provided an immunogenic composition as described herein, wherein immunogenic composition is in form of a vaccine.

In an embodiment of the present disclosure, there is provided an immunogenic composition as described herein, wherein immunogenic composition is in form of a multivalent vaccine. The multivalent vaccine comprises HA protein sequences modified by deleting and replacing the cytoplasmic domain with a linker, including but not limited to GSAG, wherein the HA protein sequences is obtained from two, three, four, or more different strains of influenza virus, as per the WHO recommended strains for which the sequences are available at GISAID database.

In an embodiment of the present disclosure, there is provided an immunogenic composition as described herein, wherein immunogenic composition is in form of quadrivalent vaccine.

In an embodiment of the present disclosure, there is provided a method for obtaining the immunogenic composition as described herein, wherein the method comprises: (a) culturing the recombinant host cell as described herein under suitable conditions to obtain the polypeptide fragment as described herein; (b) subjecting the polypeptide to purification; and (c) contacting the polypeptide of step (b) with a pharmaceutically acceptable carrier for obtaining the immunogenic composition.

In an embodiment of the present disclosure, there is provided a method for obtaining the immunogenic composition as described herein, wherein the pharmaceutically acceptable carrier is selected from the group consisting of at least one adjuvant, and excipients. In another embodiment of the present disclosure, the pharmaceutically acceptable carrier is an adjuvant selected from the group consisting of squalene-in-water emulsion (SWE) adjuvant and chemically equivalent adjuvant.

In an embodiment of the present disclosure, there is provided a method for obtaining the immunogenic composition as described herein, wherein the recombinant host cell comprising the recombinant construct or the recombinant vector comprises a nucleic acid fragment encoding a polypeptide fragment comprising an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30, wherein the recombinant host cell is an insect cell.

In an embodiment of the present disclosure, there is provided a method for eliciting an immune response to an influenza disease in a subject, the method comprising administering the subject a pharmaceutically effective amount of the immunogenic composition as described herein.

In an embodiment of the present disclosure, there is provided a method for preventing an influenza disease in a subject, the method comprising administering the subject a pharmaceutically effective amount of the immunogenic composition as described herein.

In an embodiment of the present disclosure, there is provided a method as described herein, wherein the immunogenic composition is administered by a mode selected from the group consisting of intranasal, parenteral, subcutaneous, intravenous, intra-arterial, intra-peritoneal, intramuscular, intradermal, oral, dermal, nasal, and inhalation.

In an embodiment of the present disclosure, there is provided a kit comprising the polypeptide as described herein, or the immunogenic composition as described herein, and an instruction leaflet.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Example 1

### Cloning, Expression and Purification

HA protein sequences for influenza virus strain recommendations for NH 2021-2022 (NQ21) by WHO are: A/Wisconsin/588/2019 (H1N1)pdm09-like virus; A/Cambodia/e0826360/2020 (H3N2)-like virus; B/Washington/02/2019 (B/Victoria lineage)-like virus; and B/Phuket/3073/2013 (B/Yamagata lineage)-like virus were downloaded from GISAID Epiflu database with accession numbers EPI1812046, EPI1853346, EPI1691903 and EPI1799824 respectively. A similar strategy is applied in case of HA protein sequences for influenza virus strain recommendations for NQ20 (NH 2020-21) by WHO. Similarly, a person skilled in the art can obtain the HA protein sequences from different influenza strains (WHO recommended strains) for which the sequences can be downloaded from GISAID database (GISAID—Initiative https://platform.epicov.org/epi3/frontend #554808). The numbering scheme of the HA sequences is based on the nomenclature provided by Burke and Smith (2014) (Burke D F, Smith D J. A recommended numbering scheme for influenza A HA subtypes. PLoS One. 2014 Nov. 12; 9(11):e112302. doi: 10.1371/journal.pone.0112302. PMID: 25391151; PMCID: PMC4229193) which is incorporated herein by reference in its entirety for all purposes of the present disclosure.

All HA genes and its corresponding protein sequences (obtained from different influenza strains) were codon-optimized for high-level expression in Spodoptera frugiperda (Sf9) insect cells (Thermo Fisher Scientific, Sf9 cells in Sf-900IIISFM, Cat No. 12659017) and received synthetic genes from Genscript (Piscataway, NJ, USA). The HA protein sequence having the cytoplasmic domain, obtained from influenza strain A/Wisconsin/588/2019 (H1N1)pdm09-like virus has an amino acid sequence as set forth in SEQ ID NO: 31.

For the purpose of the present disclosure, the cytoplasmic domain (CD) of the HA protein sequence obtained from different influenza strains was deleted and replaced with a linker such as GSAG, in order to get rid of the cysteine residues present in the CD. Either a complete sequence of the cytoplasmic domain is deleted from the HA protein or a small portion of the cytoplasmic domain having a length in the range of 7 to 17, or 12 to 14 amino acid residues is deleted, depending on the influenza virus strain (WHO recommended strain) being targeted in the present disclosure.

### 1.1 Cloning:

Various nucleic acid fragments (i.e., sequence of HA gene having a linker in place of cytoplasmic domain) were cloned with GP67 signal peptide sequence (encoded by SEQ ID NO: 32) into pFastBac1 vector (Thermo Fisher Scientific) between EcoRI and HindIII under Autographa californica multiple nuclear polyhedrosis virus (AcMNPV) polyhedrin promoter, to obtain different recombinant constructs. Although the linker used for replacing the cytoplasmic domain of HA gene was GSAG, however, it can be contemplated that a person skilled in the art can use any linker having a length in the range of 2 to 9 or 2 to 7 or 2 to 5 amino acid residues. It is also appreciated to use any other sequence encoding a signal peptide in the recombinant construct (apart from GP67 signal peptide) that is known to a person skilled in the art, by following the process as described in the present example.

The recombinant construct optionally comprises HRV3C recognition sequence and histidine tag sequence.

### Nucleic Acid Fragments Used for Constructing the Recombinant Constructs of the Present Disclosure

Table 1 shows the sequences of the nucleic acid fragment (HA gene having GSAG linker in place of cytoplasmic domain) obtained from different influenza strains, were used for arriving at the different recombinant constructs. The recombinant construct comprises the nucleic acid fragments represented by their respective SEQ IDs.

TABLE 1

| | SEQ ID NO of | | | |
|---|---|---|---|---|
| Recombinant | the nucleic acid | Reference | | |
| construct | fragment | strain | WHO Reference strain year | Strain name |
| sMH1_02HA0d | SEQ ID NO: 1 | H1N1 | 2020-21 NH | A/Hawaii/70/2019 |
| sMH3_02HA0d | SEQ ID NO: 3 | H3N2 | 2020-21 NH, 2021 SH | A/Hong Kong/45/2019 |
| sMHV_02HA0d | SEQ ID NO: 5 | Influenza B | 2020 SH, 2020-21 NH, | B/Washington/02/2019 |
| | | Victoria | 2021 SH, 2021-22 NH | |
| sMHY_01HA0d | SEQ ID NO: 7 | Influenza B | 2016-17 NH, 2017-18 NH, | B/Phuket/3073/2013 |
| | | Yamagata | 2018-19 NH, 2019-20 NH, | |
| | | | 2020-21 NH, 2021-22 NH, | |
| | | | 2017 SH, 2018 SH, 2019 | |
| | | | SH, 2020 SH, 2021 SH, | |
| | | | 2022 SH | |
| sMH1_02HA0d- | SEQ ID NO: 9 | H1N1 | 2020-21 NH | A/Hawaii/70/2019 |
| delHis | | | | |
| sMH3_02HA0d- | SEQ ID NO: 11 | H3N2 | 2020-21 NH, 2021 SH | A/Hong Kong/45/2019 |
| delHis | | | | |
| sMHV_02HA0d- | SEQ ID NO: 13 | Influenza B | 2020 SH, 2020-21 NH, | B/Washington/02/2019 |
| delHis | | Victoria | 2021 SH, 2021-22 NH | |
| sMHY_01HA0d- | SEQ ID NO: 15 | Influenza B | 2016-17 NH, 2017-18 NH, | B/Phuket/3073/2013 |
| delHis | | Yamagata | 2018-19 NH, 2019-20 NH, | |
| | | | 2020-21 NH, 2021-22 NH, | |
| | | | 2017 SH, 2018 SH, 2019 | |
| | | | SH, 2020 SH, 2021 SH, | |
| | | | 2022 SH | |
| sMH1_04HA0d-Tg | SEQ ID NO: 17 | H1N1 | 2021 SH, 2021-22 NH, | A/Wisconsin/588/2019 |
| | | | 2022 SH | |
| sMH3_03HA0d-Tg | SEQ ID NO: 19 | H3N2 | 2021-22 NH | A/Cambodia/e0826360/2020 |
| sMH1_03HA0d | SEQ ID NO: 21 | H1N1 | 2021 SH, 2021-22 NH, | A/Wisconsin/588/2019 |
| | | | 2022 SH | |
| sMH3_S22_HA0d | SEQ ID NO: 23 | H3N2 | 2022 SH | A/Darwin/6/2021 |
| sMH3_S22_HA0d-Tg | SEQ ID NO: 25 | H3N2 | 2022 SH | A/Darwin/6/2021 |
| sMHV_S22_HA0d | SEQ ID NO: 27 | Influenza B | 2022 SH | B/Austria/1359417/2021 |
| | | Victoria | | |
| sMHV_S22_HA0d- | SEQ ID NO: 29 | Influenza B | 2022 SH | B/Austria/1359417/2021 |
| Tg | | Victoria | | |

In addition to the nucleic acid fragment sequence, each of the recombinant constructs (as mentioned above in Table 1) also comprises a kozak sequence (GCCGCCACC), and GP67 signal peptide (SEQ ID NO:32). The recombinant constructs optionally comprise HRV3C recognition sequence, histidine tag sequence. One such example of the recombinant construct comprising nucleic acid sequence encoding signal peptide, HRV3C recognition sequence, and histidine tag sequence is sMH1_02HA0d encoded by a nucleotide sequence as set forth in SEQ ID NO: 33.

Further, each of the recombinant constructs (as mentioned in Table 1) expressing HA with deleted CD genes, having the nucleic acid sequence as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, and SEQ ID NO: 29, were cloned into Bac to Bac baculovirus expression system (Thermo Fisher Scientific), to prepare the recombinant baculovirus (recombinant vector). Sf9 cells (insect cells) propagated in Sf900III SFM media at 4 million/mL density, were infected with recombinant baculovirus expressing HA genes with deleted cytoplasmic domain, to obtain recombinant Sf9 cell or recombinant insect cells (recombinant host cell).

### 1.2 Purification of Protein

The recombinant insect cells were harvested by centrifugation, at 72 h. The proteins as expressed (which are called as recombinant proteins) by the recombinant insect cells were extracted from cell membranes with non-ionic detergent Tergitol™ NP-9 and purified with a combination of ion-exchange chromatography and affinity chromatography (lentil lectin). The final protein was then contacted with phosphate buffer saline (PBS) containing 5% Sucrose, 0.01% Tween 20 and 0.5 M NaCl, to obtain an immunogenic composition.

The description of the expressed or secreted polypeptides with His Tags and without His Tags (i.e., proteins purified by the process as described above), is provided in the Table 2.

TABLE 2

| | | | | |
|---|---|---|---|---|
| SEQ ID NO of the | | | | |
| expressed/secreted | | | | |
| polypeptide | Description of | Reference | WHO Reference | |
| fragment | the sequence | strain | strain year | Strain name |

Protein sequence with His Tags

| | | | | |
|---|---|---|---|---|
| SEQ ID NO: 2 | Depicts the amino | H1N1 | 2020-21 NH | A/Hawaii/70/2019 |
| | acid sequence of | | | |
| | the polypeptide | | | |
| | encoded by SEQ | | | |
| | ID NO: 1 | | | |
| SEQ ID NO: 4 | Depicts the amino | H3N2 | 2020-21 NH, 2021 SH | A/Hong Kong/45/2019 |
| | acid sequence of | | | |
| | the polypeptide | | | |
| | encoded by SEQ | | | |
| | ID NO: 3 | | | |
| SEQ ID NO: 6 | Depicts the amino | Influenza B | 2020 SH, 2020-21 NH, | B/Washington/02/2019 |
| | acid sequence of | Victoria | 2021 SH, 2021-22 NH | |
| | the polypeptide | | | |
| | encoded by SEQ | | | |
| | ID NO: 5 | | | |
| SEQ ID NO: 8 | Depicts the amino | Influenza B | 2016-17 NH, 2017-18 NH, | B/Phuket/3073/2013 |
| | acid sequence of | Yamagata | 2018-19 NH, 2019-20 NH, | |
| | the polypeptide | | 2020-21 NH, 2021-22 NH, | |
| | encoded by SEQ | | 2017 SH, 2018 SH, 2019 | |
| | ID NO: 7 | | SH,2020 SH, 2021 SH, | |
| | | | 2022 SH | |
| SEQ ID NO: 22 | Depicts the amino | H1N1 | 2021 SH, 2021-22 NH, | A/Wisconsin/588/2019 |
| | acid sequence of | | 2022 SH | |
| | the polypeptide | | | |
| | encoded by SEQ | | | |
| | ID NO: 21 | | | |
| SEQ ID NO: 24 | Depicts the amino | H3N2 | 2022 SH | A/Darwin/6/2021 |
| | acid sequence of | | | |
| | the polypeptide | | | |
| | encoded by SEQ | | | |
| | ID NO: 23 | | | |
| SEQ ID NO: 28 | Depicts the amino | Influenza B | 2022 SH | B/Austria/1359417/2021 |
| | acid sequence of | Victoria | | |
| | the polypeptide | | | |
| | encoded by SEQ | | | |
| | ID NO: 27 | | | |

Protein sequences without His tags

| | | | | |
|---|---|---|---|---|
| SEQ ID NO: 10 | Depicts the amino | H1N1 | 2020-21 NH | A/Hawaii/70/2019 |
| | acid sequence of | | | |
| | the polypeptide | | | |
| | encoded by SEQ | | | |
| | ID NO: 9 | | | |
| SEQ ID NO: 12 | Depicts the amino | H3N2 | 2020-21 NH, 2021 SH | A/Hong Kong/45/2019 |
| | acid sequence of | | | |
| | the polypeptide | | | |
| | encoded by SEQ | | | |
| | ID NO: 11 | | | |
| SEQ ID NO: 14 | Depicts the amino | Influenza B | 2020 SH, 2020-21 NH, | B/Washington/02/2019 |
| | acid sequence of | Victoria | 2021 SH, 2021-22 NH | |
| | the polypeptide | | | |
| | encoded by SEQ | | | |
| | ID NO: 13 | | | |
| SEQ ID NO: 16 | Depicts the amino | Influenza B | 2016-17 NH, 2017-18 NH, | B/Phuket/3073/2013 |
| | acid sequence of | Yamagata | 2018-19 NH, 2019-20 NH, | |
| | the polypeptide | | 2020-21 NH, 2021-22 NH, | |
| | encoded by SEQ | | 2017 SH, 2018 SH, 2019 | |
| | ID NO: 15 | | SH, 2020 SH, 2021 SH, | |
| | | | 2022 SH | |
| SEQ ID NO: 18 | Depicts the amino | H1N1 | 2021 SH, 2021-22 NH, | A/Wisconsin/588/2019 |
| | acid sequence of | | 2022 SH | |
| | the polypeptide | | | |
| | encoded by SEQ | | | |
| | ID NO: 17 | | | |
| SEQ ID NO: 20 | Depicts the amino | H3N2 | 2021-22 NH | A/Cambodia/e0826360/2020 |
| | acid sequence of | | | |
| | the polypeptide | | | |
| | encoded by SEQ | | | |
| | ID NO: 19 | | | |
| SEQ ID NO: 26 | Depicts the amino | H3N2 | 2022 SH | A/Darwin/6/2021 |
| | acid sequence of | | | |
| | the polypeptide | | | |
| | encoded by SEQ | | | |
| | ID NO: 25 | | | |
| SEQ ID NO: 30 | Depicts the amino | Influenza B | 2022 SH | B/Austria/1359417/2021 |
| | acid sequence of | Victoria | | |
| | the polypeptide | | | |
| | encoded by SEQ | | | |
| | ID NO: 29 | | | |

The purification of the polypeptide fragment involves the removal of His tags. The purified proteins derived by cleaving a His tag sequence (amino acid sequence as set forth in SEQ ID NO: 37), has a protein sequence ending with either: (i) a linker, such as GSAG (as in the case of the protein sequences having an amino acid sequence as set forth in SEQ ID NO: 10, SEQ ID NO: 12, SEQ ID NO: 14, SEQ ID NO: 16, SEQ ID NO: 18, SEQ ID NO: 20, SEQ ID NO: 26, and SEQ ID NO: 30); or (ii) HRV3C recognition sequence (SEQ ID NO: 35), downstream of a linker (GSAG) present in SEQ ID NO: 10, SEQ ID NO: 12, SEQ ID NO: 14, SEQ ID NO: 16, SEQ ID NO: 18, SEQ ID NO: 20, SEQ ID NO: 26, and SEQ ID NO: 30. It can be concluded that these purified protein sequence ending with either a linker (GSAG), or HRV3C recognition sequence downstream of the linker, both can be used in the immunogenic composition.

The immunogenic composition as described in the present example is in form of a vaccine that also comprises squalene-in-water emulsion (SWE) adjuvant or AddaVax™ adjuvant. In particular, the immunogenic composition is a multivalent vaccine composition comprising that target against various influenza strains ((WHO recommended influenza strains). A person skilled in the art can also any other adjuvants known in the art as an alternative.

### 1.3 Polypeptide (Vaccine Candidates) Disclosed in the Present Disclosure

The combination of polypeptide fragments that is used for preparing the immunogenic composition is described in the present example. The present disclosure provides an immunogenic composition (NQ20) consisting of a combination of four polypeptide fragments having an amino acid sequence as set forth in SEQ ID NO: 10, SEQ ID NO: 12, SEQ ID NO: 14, SEQ ID NO: 16, and an adjuvant (SWE adjuvant or AddaVax™ adjuvant), wherein the immunogenic composition elicits immune response against the four reference strains, namely H1N1, H3N2, Influenza B Yamagata, and Influenza B Victoria. The NQ20 immunogenic composition is a quadrivalent immunogenic composition containing the SEQ ID NO: 10, SEQ ID NO: 12, SEQ ID NO: 14, SEQ ID NO: 16, where the HA protein sequence was derived from four influenza strains-A/Hawaii/70/2019, A/Hong Kong/45/2019, B/Washington/02/2019, and B/Phuket/3073/2013 (as per WHO recommended strains for 2020-2021 influenza season), and wherein the HA protein sequence was modified by deletion and replacement of cytoplasmic domain with GSAG linker.

Another combination of four polypeptide fragments used for preparing the immunogenic composition is also described in the present example. The immunogenic composition (NQ21) consisting of a combination of four polypeptide fragments having an amino acid sequence as set forth in SEQ ID NO: 14, SEQ ID NO: 16, SEQ ID NO: 18, and SEQ ID NO: 20, that was derived from HA protein sequences of four influenza virus strains—B/Washington/02/2019, B/Phuket/3073/2013, A/Wisconsin/588/2019, A/Cambodia/e0826360/2020 (as per WHO recommended strains for 2021-2022 influenza season), were also used for immunization purposes.

Hence, both the NQ20 and NQ21 immunogenic compositions (test immunogenic compositions) works effectively as a quadrivalent vaccines

Although the HA protein sequences were obtained from the influenza strains as shown in Table 1 and 2 and modified by replacing the cytoplasmic domain with a linker, however, it can be contemplated that a person skilled in the art can use other WHO recommended influenza strains for obtaining HA protein for which the sequences are available at GISAID database. The cytoplasmic domain of said HA protein sequence is then deleted and replaced with a linker, to obtain various other polypeptide fragments that can be further used in combination to obtain the immunogenic composition targeting multiple strains of influenza virus (WHO recommended influenza strains).

### Example 2

### Immunization Studies

### Mice Immunization

Group of 5, female, Balb/c mice, (6-8 weeks old, approximately weighing 17-18 g) were immunized with equal amounts of each purified HA-delCD protein (NQ20) having amino acid sequence as set forth in SEQ ID NO: 10, SEQ ID NO: 12, SEQ ID NO: 14, and SEQ ID NO: 16, respectively, or each purified HA-delCD protein (NQ21) having amino acid sequence as set forth in SEQ ID NO: 14, SEQ ID NO: 16, SEQ ID NO: 18, and SEQ ID NO: 20, diluted in 50 μl phosphate-buffered saline (PBS, pH 7.4), and mixed with 50 μl of AddaVax™ adjuvant (vac-adx-10) (1:1 v/v Antigen:AddaVax™ ratio per animal/dose) (InvivoGen, USA) or SWE (Seppic, USA). Immunizations were given by intramuscular injection on Day 0 (prime) and 21 (boost). Blood was collected, and serum isolated on day −2 (pre-bleed), 14 and 35, following the prime and boost immunization, respectively. Mice were challenged on Day 42 with the mouse adopted viruses.

### Guinea Pigs Immunization

Group of 10, female, Hartley strain guinea pigs, (6-8 weeks old, approximately weighing 300 g) were immunized with 20 μg purified equal amounts of each purified HA-delCD protein (NQ20), having amino acid sequence as set forth in SEQ ID NO: 10, SEQ ID NO: 12, SEQ ID NO: 14, and SEQ ID NO: 16, respectively, or each purified HA-delCD protein (NQ21) having amino acid sequence as set forth in SEQ ID NO: 14, SEQ ID NO: 16, SEQ ID NO: 18, and SEQ ID NO: 20, diluted in 50 μl phosphate-buffered saline (PBS, pH 7.4), and mixed with 50 μl of AddaVax™ adjuvant (vac-adx-10) (1:1 v/v Antigen:AddaVax™ ratio per animal/dose) (InvivoGen, USA). Immunizations were given by intramuscular injection on Day 0 (prime) and 21 (boost). Blood was collected, and serum isolated on day −2 (pre-bleed), 14 and 35, following the prime and boost immunization, respectively.

### Example 3

### Assays for Determination of Serum Antibody Titers

### 3.1 Enzyme Linked Immunosorbent Assay (ELISA)

Enzyme linked immunosorbent assay (ELISA) was performed to determine serum antibody titers against test immunogens: (1)test immunogenic composition or NQ20 consisting of a combination of four polypeptide fragment having an amino acid sequence as set forth in SEQ ID NO: 10, SEQ ID NO: 12, SEQ ID NO: 14, and SEQ ID NO: 16, or (2) test immunogenic composition or NQ21 consisting of a combination of four polypeptide fragment having an amino acid sequence as set forth in SEQ ID NO: 14, SEQ ID NO: 16, SEQ ID NO: 18, and SEQ ID NO: 20. Briefly, 4 μg/mL of test immunogens (50 μL/well) were coated on 96 well Nunc plates (Thermo Fisher Scientific, Rochester, NY, USA) for 2 h at room temperature, under constant shaking at 300 rpm on a MixMate thermomixer (Eppendorf AG, Hamburg, Germany). After washing with 1×PBS containing 0.05% Tween 20 (PBST) four times, plates were blocked with 3% skimmed milk in PBST (blocking buffer) for 1 h at room temperature. Antisera raised against test immunogens were serially diluted four-fold in blocking buffer and were added to wells. Plates were then incubated for 1 h, 300 rpm at room temperature, followed by three washes with PBST, after which 50 μL of ALP-conjugated goat anti-mouse IgG secondary antibody (1:5000) diluted in blocking buffer was added (50 μL/well) and incubated at room temperature for 1 h. Plates were washed with PBST (four times) followed by the addition of pNPP liquid (SIGMA-ALDRICH) substrate (50 μL) to each well and then incubated at 37° C. for 30 min. Optical density at 405 nm was measured. The highest serum dilution possessing signal above 0.2 (O. D at 405 nm) was considered the endpoint titer for ELISA. Data were plotted using Prism v8.4.3 (Graph Pad Software, San Diego, CA, USA). Two-tailed Student's t-test was performed for pairwise ELISA endpoint titer comparisons.

### 3.2 Hemagglutinin Inhibition (HI) Assay

HI titers to vaccine-matched and challenge-matched viruses were tested with mice sera. Immunized mice sera were heat-inactivated and treated with receptor destroying enzyme (RDE, SIGMA-ALDRICH) before use. Sera were two-fold serially diluted with cold PBS buffer and incubated with the indicated viruses (4 HAU/well), and then incubated with 1% Guinea pig red blood cells (RBC) for 30 min at room temperature. Hemagglutinin inhibition (HI) titers were recorded as the highest serum dilution at which no agglutination was observed. Two-tailed Student's t-test was performed for pairwise HI titer comparisons.

### 3.3 Microneutralization Assay

Viruses were grown in Madin-Darby Canine Kidney (MDCK) cells in the presence of TPCK-treated trypsin (1 μg/mL) and stored at −70° C. Immune mice sera samples were heat-inactivated and treated with receptor destroying enzyme (RDE, SIGMA-ALDRICH) before use. Immunized sera samples were two-fold serially diluted and incubated for 1 h at 37° C. in 5% CO₂ with 50 TCID50 viruses. Serum-virus mixture was then transferred to 96 well plates, and 1.5×10⁵ MDCK-London cells/mL were added to each well. Plates were then incubated for 48 h at 37° C. in 5% CO₂, and cytopathic effects were observed. MN assay for matched virus was only performed with group I, III, V (H1) and suspended in DMEM serum free media. The neutralization titer in the assay is the highest serum dilution at which no cytopathic effect was observed. Two-tailed Student's t-test was performed for pairwise MN titer comparisons.

### Results

### (A) ELISA Assay and Neutralization Assay: Mice

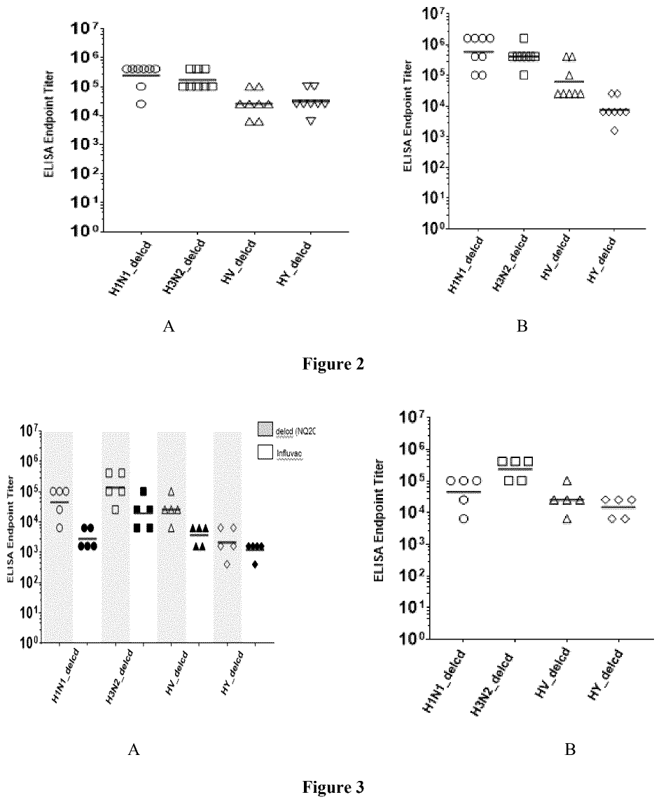

FIG. 2 shows the results of ELISA and Table 3 summarizes the results of HI assay showing binding titers values against the HA antigens of the following reference influenza virus strains: H1N1, H3N2, Influenza B Victoria, and Influenza B Yamagata, in the sera (collected on day 35) of mice immunized with the vaccine candidate (test immunogenic composition; N20) adjuvanted with AddaVax™, or SWE adjuvant, respectively.

TABLE 3

| | A/Guangdong- | | A/Singapore/ | | | | A/Guangdong- |
|---|---|---|---|---|---|---|---|
| | Maonan/ | A/Honkong/ | INFIM-16- | A/Kansas/ | B/Washington/ | B/Phuket/ | Maonan/ |
| | SWL1536/2019 | 2671/2019 | 0019/2016 | 12/2017 | 02/2019 | 3073/2013 | SWL1536/2019 |
| Group | (H1N1) | H3N2 | (H3N2) | (H3N2) | (Victoria) | (Yamagata) | (H1N1) |
| | HI | HI | HI | HI | HI | HI | Virus |
| | | | | | | | Neutralization |
| | Matched | Matched | Mis- | Mis- | Matched | Matched | Matched |
| | | | Matched | Matched | | | |
| Test immunogenic | 1076 | 830 | 494 | 123 | 174 | 320 | 905 |
| composition-addavax | | | | | | | |
| Test immunogenic | 987 | 830 | 538 | 293 | 174 | 293 | 761 |
| composition-SWE | | | | | | | |

Boost sera collected on day 35 showed geometric mean ELISA titer and HI titer values with both matched and mismatched viruses of four strains H1N1, H3N2, Influenza B Victoria, and Influenza B Yamagata, in mice administered with the vaccine candidate or the test immunogenic composition (NQ 20) adjuvanted with AddaVax™ (as shown in Table 3 and FIG. 2A), or SWE adjuvant (as show in Table 3 and FIG. 2B).

### (B) ELISA Assay: Guinea Pigs

FIG. 3 depicts the results of ELISA assay showing binding titers values against the HA antigens of the following reference influenza virus strains: H1N1, H3N2, Influenza B Victoria, and Influenza B Yamagata in the sera (collected on day 35) of Guinea Pigs immunized with the vaccine candidate or test immunogenic compositions (NQ20(adjuvanted with AddaVax™, or SWE adjuvant, respectively.

High ELISA titer values against all four strains of influenza virus H1N1, H3N2, Influenza B Victoria, and Influenza B Yamagata, indicates that an enhanced immune response was elicited in Guinea Pigs immunized with the vaccine candidate or the test immunogenic composition (NQ20) adjuvanted with AddaVax™ (FIG. 3A), and SWE adjuvant (FIG. 3B), respectively.

Thus, it can be inferred from the above observation, that the test composition or the vaccine candidate (NQ20 and NQ21) adjuvanted with AddaVax™, or SWE adjuvant, shows higher ELISA titer value as well as HI titer value against all four strains of influenza virus H1N1, H3N2, Influenza B Victoria, and Influenza B Yamagata when administered in mice or guinea pigs, thus providing significant protection to them against the influenza infection. This indicates, that the immunogenic composition is a quadrivalent vaccine composition that provides significant protective immune response against four strains of influenza virus (H1N1, H3N2, Influenza B Victoria, and Influenza B Yamagata) in both mice and guinea pigs.

### Example 4

### Comparison with Conventional Flu Vaccines

In order to evaluate the effectivity of the test immunogenic composition to induce the immune response against the different strains of influenza virus, it's immunogenicity was compared with the immunogenicity of one of the commercial vaccine, i.e., Influvac (an egg-grown, inactivated viral vaccine containing full-length HA protein having cytoplasmic domain) from Abbott laboratories, as recommended by WHO for 2020-2021 influenza season.

For this purpose, mice were immunized with the test immunogenic composition (SEQ ID NO: 10+SEQ ID NO: 12+SEQ ID NO: 14+SEQ ID NO: 16) and Influvac, separately. The immunogenic composition (3, 15, 20 or 45 mcg dose) was mixed in equal volumes of AddaVax™, or SWE adjuvants. Similarly, Influfac was mixed AddaVax™, or SWE adjuvants. Immunizations were given by intramuscular injection on Day 0 (prime) and 21 (boost). Blood was collected, and serum isolated on day −2 (pre-bleed), 14 (Interim bleed) and 35 (Post Boost bleed), following the prime and boost immunization, respectively. This immunization schedule was further followed by a virus challenge on day 42. Following virus challenge, the sera of mice was collected to determine the HI titer values and neutralization titer values, by following the process as described in example 3. Table 4 shows the comparison of HI titer values and virus-neutralizing (VN) titer values between the test immunogenic composition of the present disclosure and Influvac.

TABLE 4

| | A/Guangdong- | | A/Singapore/ | | | | A/Guangdong- |
|---|---|---|---|---|---|---|---|
| | Maonan/ | A/Honkong/ | INFIM-16- | | | B/Phuket/3073/ | Maonan/ |
| | SWL1536/ | 2671/2019 | 0019/2016 | A/Kansas/14/ | B/Washington/02/ | 2013 | SWL1536/2019 |
| Group | 2019 (H1N1) | H3N2 | (H3N2) | 2017 (H3N2) | 2019 (Victoria) | (Yamagata) | (H1N1) |
| | HI | HI | HI | HI | HI | HI | Virus Neutralization |
| | Matched | Matched | Mis-Matched | Mis-Matched | -Matched | Matched | Matched |
| Test | 1076 | 830 | 494 | 123 | 174 | 320 | 905 |
| immunogenic | | | | | | | |
| composition - | | | | | | | |
| addavax | | | | | | | |
| Test | 987 | 830 | 538 | 293 | 174 | 293 | 761 |
| immunogenic | | | | | | | |
| composition - | | | | | | | |
| SWE | | | | | | | |
| Influvac | 343 | 184 | 75 | 57 | 171 | 61 | 485 |
| (Commercial | | | | | | | |
| vaccine) | | | | | | | |

From Table 4, it is clear that the boost sera of mice collected on day 35 showed high geometric mean HI & VN titers with both matched and mis-matched viruses, upon immunization with the test immunogenic composition (NQ20) (vaccine candidate) of the present disclosure. It can also be observed from Table 4 that the test immunogenic composition (NQ20: SEQ ID NO: 10+SEQ ID NO: 12+SEQ ID NO: 14+SEQ ID NO: 16) shows significantly higher HI and virus-neutralizing (VN) titer values as compared to that of influvac vaccine containing full length HA protein with cytoplasmic domain. This clearly demonstrates that the immunogenic composition of the present disclosure has better immunogenic properties against the different strains of influenza virus as compared to that of influvac.

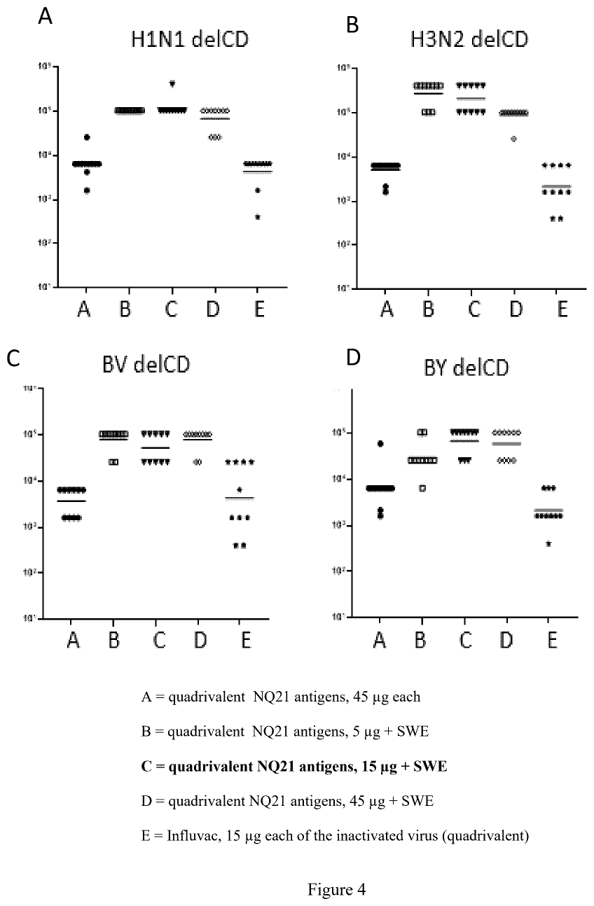

Similar immunogenic studies were conducted with guinea pigs that were immunized with test immunogenic composition (NQ21) (SEQ ID NO: 14+SEQ ID NO: 16+SEQ ID NO: 18+SEQ ID NO: 21) and Influvac, separately. As shown in FIG. 4, the following doses of the immunogenic composition was administered to the guinea pigs: (A): 45 μg of each of the polypeptide fragments having SEQ ID NO: 14, SEQ ID NO: 16, SEQ ID NO: 18, SEQ ID NO: 20 (without adjuvant); (B): 5 μg of each of the polypeptide fragments having SEQ ID NO: 14, SEQ ID NO: 16, SEQ ID NO: 18, SEQ ID NO: 20 mixed with SWE adjuvant; (C): 15 μg of each of the polypeptide fragments having SEQ ID NO: 14, SEQ ID NO: 16, SEQ ID NO: 18, SEQ ID NO: 20 mixed with SWE adjuvant; and (D) 45 μg of each of the polypeptide fragments having SEQ ID NO: 14, SEQ ID NO: 16, SEQ ID NO: 18, SEQ ID NO: 20 mixed with SWE adjuvant. For comparative purposes, guinea pigs were immunized with 15 μg of each of the inactivated viruses presented quadrivalent influvac vaccine (as indicated with E in FIG. 4).

Table 5 shows the comparison of HI titer values between the test immunogenic composition of the present disclosure and Influvac.

TABLE 5

| | | | | | B/Colorado/ | | A/Kansas/ |
|---|---|---|---|---|---|---|---|
| | A/Wisconsin/ | A/Cambodia/ | B/Washington/ | B/Phuket/ | 06/2017- | A/Brisbane/ | 14/2017 |
| | 588/2019 | e0826360/ | 02/2019 | 3073/2013 | like virus | 02/2018 | (H3N2)- |
| Group | (H1N1)pdm09- [symbol] | 2020 (H3N2)- [symbol] | (B/Victoria [symbol] | (B/Yamagata [symbol] | (B/Victor [symbol] | (H1N1)pdm09- [symbol] | like virus |
| | HI | HI | HI | HI | HI | HI | HI |
| | Matched | Matched | Matched | Matched | Mis- | Mis- | Mis- |
| | | | | | Matched | Matched | Matched |
| HA_delCD-45 mcg | 160 | 98 | 53 | 75 | 121 | 80 | 35 |
| unadjuvanted | | | | | | | |
| HA_delCD-5 mcg + | 1114 | 735 | 279 | 368 | 184 | 485 | 343 |
| SWE | | | | | | | |
| HA_delCD-15 mcg + | 1114 | 905 | 229 | 320 | 139 | 597 | 394 |
| SWE | | | | | | | |
| HA_delCD-45 mcg + | 788 | 788 | 197 | 260 | 106 | 557 | 299 |
| SWE | | | | | | | |
| Influvac | 735 | 279 | 43 | 121 | 32 | 243 | 149 |

[symbol]

indicates data missing or illegible when filed

It can be inferred that boost sera of guinea pigs collected on day 35 showed high ELISA titer values when immunized with the test immunogenic composition NQ21, adjuvanted with SWE adjuvant (FIG. 4, Table 5), as compared to that of influvac.

Hence, the immunogenic compositions: NQ20 (SEQ ID NO: 10+SEQ ID NO: 12+SEQ ID NO: 14+SEQ ID NO: 16) and NQ21 (SEQ ID NO: 14, SEQ ID NO: 16, SEQ ID NO: 18, SEQ ID NO: 20) of the present disclosure, is in a form of a quadrivalent vaccine composition eliciting enhanced immune response against four influenza strains (H1N1, H3N2, Influenza B Victoria, and Influenza B Yamagata) in both mice and guinea pigs, as compared to that of influvac.

Overall, the present disclosure discloses a polypeptide fragment comprising HA protein sequence with deleted cytoplasmic domain, and wherein said cytoplasmic domain is replaced with a linker, like GSAG or GSSAG or similar linkers. Although the polypeptide fragment as disclosed herein comprises an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30, however, it can be contemplated that a person skilled in the art can obtain HA protein sequences from different strains of influenza virus (as per the recommendations of World Health Organisation (WHO), for which the HA DNA and protein sequences are available at GISAID database, and can modify the said HA protein sequence by deleting and replacing the cytoplasmic domain with a linker to get rid of cysteine residues. The examples of different WHO recommended influenza strains from which the HA protein sequence is obtained are provided in Tables 6 and 7. The polypeptide fragment as disclosed herein was further used in an immunogenic composition that elicited enhanced immune response in a subject (for instance, mice and guinea pigs) with high ELISA titer values, high HI titer values, and high VN titer values as compared to a commercial flu vaccine (influvac).

TABLE 6
Northern hemisphere

| | | | | |
|---|---|---|---|---|
| 2016-2017 | A/California/7/2009 | A/Hong Kong/4801/2014 | B/Brisbane/60/2008- | B/Phuket/3073/2013- |
| | (H1N1)pdm09-like | (H3N2)-like virus | like virus | like virus |
| | virus | | | |
| 2017-2018 | A/Michigan/45/2015 | A/Hong Kong/4801/2014 | B/Brisbane/60/2008- | B/Phuket/3073/2013- |
| | (H1N1)pdm09-like | (H3N2)-like virus | like virus | like virus |
| | virus | | | |
| 2018-2019 | A/Michigan/45/2015 | A/Singapore/INFIMH- | B/Colorado/06/2017- | B/Phuket/3073/2013- |
| | (H1N1)pdm09-like | 16-0019/2016 | like virus | like virus |
| | virus | (H3N2)-like virus | (B/Victoria/2/87 | (B/Yamagata/16/88 |
| | | | lineage) | lineage) |
| 2019-2020 | A/Brisbane/02/2018 | A/Kansas/14/2017 | B/Colorado/06/2017- | B/Phuket/3073/2013- |
| | (H1N1)pdm09-like | (H3N2)-like virus | like virus | like virus |
| | virus | | (B/Victoria/2/87 | (B/Yamagata/16/88 |
| | | | lineage) | lineage) |
| 2020-2021 | A/Hawaii/70/2019 | A/Hong Kong/45/2019 | B/Washington/02/2019 | B/Phuket/3073/2013 |
| cell- or | (H1N1)pdm09-like | (H3N2)-like virus | (B/Victoria lineage)- | (B/Yamagata lineage)- |
| recombinant- | virus-like virus | | like virus | like virus |
| based | | | | |
| vaccines | | | | |
| 2021-2022 | A/Wisconsin/588/2019 | A/Cambodia/e0826360/2020 | B/Washington/02/2019 | B/Phuket/3073/2013 |
| cell- or | (H1N1)pdm09-like | (H3N2)-like virus | (B/Victorialineage)- | (B/Yamagata lineage)- |
| recombinant- | virus-like virus | | like virus | like virus |
| based | | | | |
| vaccines | | | | |

TABLE 7
Southern Hemisphere

| | | | | |
|---|---|---|---|---|
| 2017 | A/Michigan/45/2015 | A/Hong Kong/4801/2014 | B/Brisbane/60/2008-like | B/Phuket/3073/2013- |
| | (H1N1)pdm09-like | (H3N2)-like virus | virus | like virus |
| | virus | | | |
| 2018 | A/Michigan/45/2015 | A/Singapore/INFIMH- | B/Brisbane/60/2008-like | B/Phuket/3073/2013- |
| | (H1N1)pdm09-like | 16-0019/2016 | virus | like virus |
| | virus | (H3N2)-like virus | | |
| 2019 | A/Michigan/45/2015 | A/Switzerland/8060/2017 | B/Colorado/06/2017-like | B/Phuket/3073/2013- |
| | (H1N1)pdm09-like | (H3N2)-like virus | virus (B/Victoria/2/87 | like virus |
| | virus | | lineage) | (B/Yamagata/16/88 |
| | | | | lineage) |
| 2020 | A/Brisbane/02/2018 | A/South Australia/34/2019 | B/Washington/02/2019- | B/Phuket/3073/2013-like |
| | (H1N1)pdm09-like | (H3N2)-like virus | like (B/Victoria | (B/Yamagata lineage) |
| | virus | | lineage) virus | virus |
| 2021 cell-or | A/Wisconsin/588/2019 | A/Hong Kong/45/2019 | B/Washington/02/2019 | B/Phuket/3073/2013 |
| recombinant- | (H1N1)pdm09-like | (H3N2)-like virus | (B/Victoria lineage)- | (B/Yamagata lineage)- |
| based | virus | | like virus | like virus |
| Vaccines | | | | |

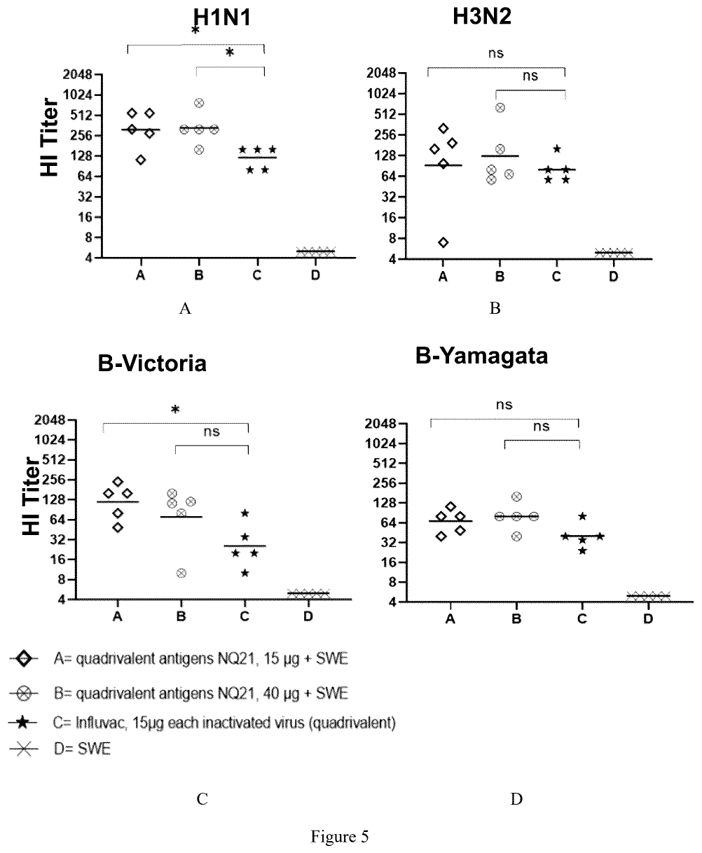

Further, immunogenic studies were conducted with hamster and ferrets that were immunized with test immunogenic composition (NQ21) (SEQ ID NO: 14+SEQ ID NO: 16+SEQ ID NO: 18+SEQ ID NO: 21) and Influvac, separately. As shown in FIG. 5, the following doses of the immunogenic composition was administered to the ferrets: (A) 15 μg of each of the polypeptide fragments having SEQ ID NO: 14, SEQ ID NO: 16, SEQ ID NO: 18, SEQ ID NO: 20 mixed with SWE adjuvant; (B) 40 μg of each of the polypeptide fragments having SEQ ID NO: 14, SEQ ID NO: 16, SEQ ID NO: 18, SEQ ID NO: 20 mixed with SWE adjuvant; (C) 15 μg of each of the inactivated viruses presented quadrivalent influvac vaccine; (D) SWE Adjuvant only. The immune response was studied against four influenza strains (H1N1(FIG. 5A), H3N2 (FIG. 5B), Influenza B Victoria (FIG. 5C), and Influenza B Yamagata (FIG. 5D)). The immunization schedule in ferrets was as follow: Day −6 (Pre-bleed), Day 0 (Prime), Day 21 (Interim bleed and Boost), Day 49 (Post Boost bleed and Viral Challenge).

Table 8 shows the comparison of HI titer values, in ferrets, between the test immunogenic composition of the present disclosure and Influvac.

TABLE 8

| | HI Matched | | | |
|---|---|---|---|---|
| | | A/Cambodia/ | | |
| | A/Victoria/ | e0826360/ | B/Washington/ | B/Phuket/ |
| | 2570/2019 | 2020 | 02/2019 | 3073/2013 |
| Groups | H1N1 | H3N2 | B-Vic | B-Yam |
| HA_delCD-15 | 314.4 | 92.8 | 119.2 | 67.7 |
| μg + SWE | | | | |
| HA_delCD-40 | 333.3 | 126.4 | 70.5 | 80 |
| μg + SWE | | | | |
| HA_delCD- | 382.8 | 81.1 | 69.6 | 45.9 |
| 15 μg + | | | | |
| Stems + SWE | | | | |
| Influvac | 121.3 | 80.2 | 25.7 | 40.4 |
| SWE only | 5 | 5 | 5 | 5 |

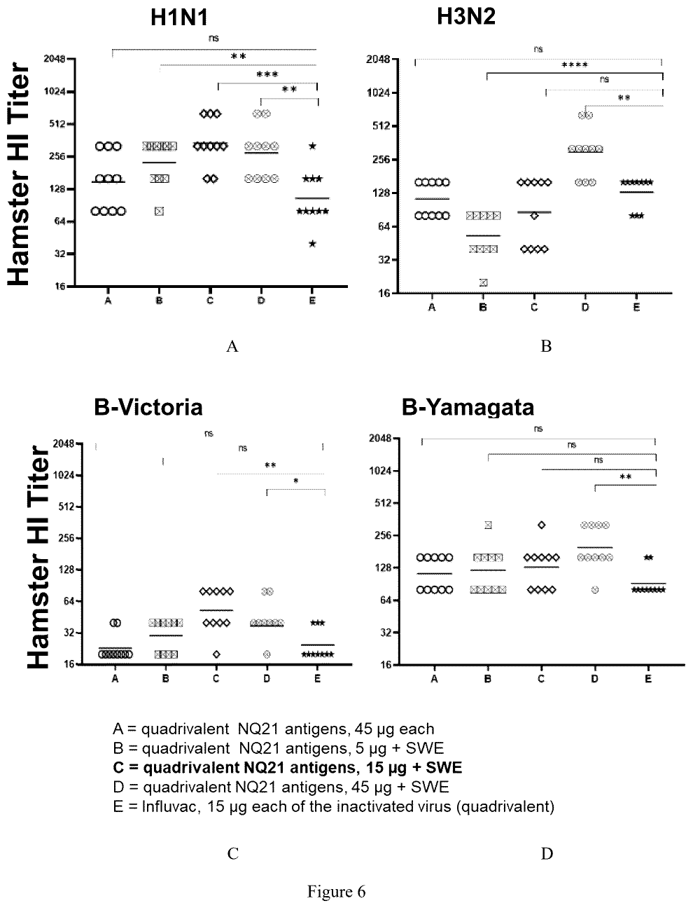

In hamster, immunization was with test immunogenic composition (NQ21) (SEQ ID NO: 14+SEQ ID NO: 16+SEQ ID NO: 18+SEQ ID NO: 21) and Influvac, separately, as shown in FIG. 6, of the following doses of the immunogenic composition: (A): 45 μg of each of the polypeptide fragments having SEQ ID NO: 14, SEQ ID NO: 16, SEQ ID NO: 18, SEQ ID NO: 20 (without adjuvant); (B): 5 μg of each of the polypeptide fragments having SEQ ID NO: 14, SEQ ID NO: 16, SEQ ID NO: 18, SEQ ID NO: 20 mixed with SWE adjuvant; (C): 15 μg of each of the polypeptide fragments having SEQ ID NO: 14, SEQ ID NO: 16, SEQ ID NO: 18, SEQ ID NO: 20 mixed with SWE adjuvant; and (D) 45 μg of each of the polypeptide fragments having SEQ ID NO: 14, SEQ ID NO: 16, SEQ ID NO: 18, SEQ ID NO: 20 mixed with SWE adjuvant. For comparative purposes, hamsters were immunized with 15 μg of each of the inactivated viruses presented quadrivalent influvac vaccine (as indicated with E in FIG. 6). The immune response was studied against four influenza strains (H1N1 (FIG. 6A), H3N2 (FIG. 6B), Influenza B Victoria (FIG. 6C), and Influenza B Yamagata (FIG. 6D)). The immunization schedule in hamsters was as follow: on day −2 (Pre-bleed), on day 0 (Prime), on day 14 (Interim bleed), on day 21 (Boost), on day 35 (Post Boost bleed).

Table 9 shows the comparison of HI titer values, in hamster, between the test immunogenic composition of the present disclosure and Influvac.

TABLE 9

| | HI Matched | | | |
|---|---|---|---|---|
| | | A/Cambodia/ | | |
| | A/Wisconsin/ | e0826360/ | B/Washington/ | B/Phuket/ |
| | 588/2019 | 2020 | 02/2019 | 3073/2013 |
| Groups | H1N1 | H3N2 | B-Vic | B-Yam |
| HA_delCD- | 149.3 | 113.1 | 23 | 113.1 |
| 45 μg | | | | |
| unadjuvanted | | | | |
| HA_delCD- | 226.3 | 52.8 | 30.3 | 121.3 |
| 5 μg + | | | | |
| SWE | | | | |
| HA_delCD- | 343 | 85.7 | 60.6 | 130 |
| 15 μg + | | | | |
| SWE | | | | |
| HA_delCD- | 278.6 | 298.6 | 42.9 | 197 |
| 45 μg + | | | | |
| SWE | | | | |
| Influvac-15 | 105.6 | 130 | 28.3 | 91.9 |
| μg | | | | |

Results: FIG. 5 depicts the results of immunogenic studies in ferrets. It was observed that the test composition, according to embodiments herein, elicited higher H1N1 (FIG. 5A), BV (FIG. 5C) HI Titers than Influvac for all tested viruses at the same antigen dose of 15 μg per HA. FIG. 6 depicts the results of immunogenic studies in hamsters. It was observed that the test composition, according to embodiments herein, elicited significantly higher H1N1 (FIG. 6A) and BV (FIG. 6C) HI Titers than Influvac for all tested viruses at the same antigen dose of 15 μg.

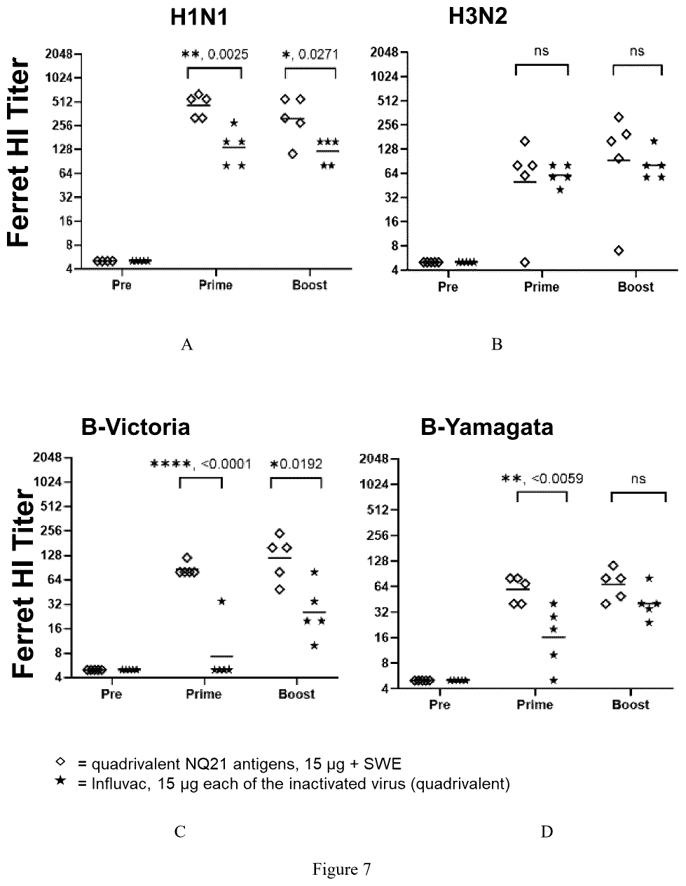

FIG. 7 depicts the results of immune response in ferrets on day 2 (pre), day 0 (prime) and day 21 (boost), of quadrivalent NQ21 antigens at 15 μg+SWE and Influvac, 15 μg each of the inactivated virus (quadrivalent). FIG. 7 depicts the immune response against four influenza strains (H1N1(FIG. 7A), H3N2 (FIG. 7B), Influenza (Flu) B Victoria (FIG. 7C), and Influenza (Flu) B Yamagata (FIG. 7D)). It is observed that the test composition, according to embodiments herein, elicits higher H1N1(FIG. 7A), BV (FIG. 7C) and BY (FIG. 7D) HI Titers than Influvac for all tested viruses after a single dose of immunization, at the same antigen dose of 15 μg per HA.

It is observed that the test immunogenic composition, according to embodiments herein, elicits higher H1N1, BV HI Titers than egg grown IIV at similar dose of 15 μg per HA. The test immunogenic composition elicits higher H1N1, BV and BY HI Titers than egg grown IIV for all tested viruses with just a single dose of immunization.

Hence, the immunogenic compositions: NQ21 (SEQ ID NO: 14, SEQ ID NO: 16, SEQ ID NO: 18, SEQ ID NO: 20) of the present disclosure elicit enhanced immune response against four influenza strains (H1N1, H3N2, Flu B Victoria, and Flu B Yamagata) in both ferrets and hamsters, as compared to that of influvac.

### Advantages of the Present Disclosure

The present disclosure discloses a recombinant influenza immunogenic composition that is based on hemagglutinin (HA) protein sequence of different influenza strains with deleted cytoplasmic domain, wherein the cytoplasmic domain is replaced with a linker, such as GSAG or GSSAG. The present disclosure discloses a polypeptide fragment having amino acid sequence as set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30. The present disclosure also discloses various recombinant construct comprising a nucleic acid fragment encoding the polypeptide fragment as described herein. The recombinant constructs or the recombinant vector comprising the recombinant construct is further used for transfecting a host cell (an insect cell) to obtain the recombinant host cell. The recombinant host cell is cultured under the suitable conditions to express the polypeptide fragment as described herein, wherein the said polypeptide fragment is subjected to purification, and subsequently mixed with an adjuvant, including but not limited to squalene-in-water emulsion (SWE) adjuvant or chemically equivalent adjuvant (for eg: Addavax), to obtain an immunogenic composition. This immunogenic composition is in a form of a recombinant HA based vaccine that elicits an enhanced immune response against various strains of influenza virus (as per WHO recommended strains). It is well demonstrated in Example 4 that the immunogenic composition shows significantly higher ELISA titer values, and higher HI and VN titer values as compared to a commercial flu vaccine (influvac). Thus, the present disclosure provides advantages of a vaccine candidate protein with a deletion in the cytoplasmic domain of HA protein of influenza virus that exhibits enhanced immunogenic properties against different strains of influenza virus, that may arise time to time. Another advantage of the deletion of the cytoplasmic domain is that the cytoplasmic tail domain contains several Cysteine residues which can potentially result in non-specific disulphide bonded aggregates.

1. A polypeptide fragment comprising a polypeptide of a modified hemagglutinin protein, wherein the modified hemagglutinin protein comprises a linker peptide replacing a polypeptide in a cytoplasmic domain of an unmodified hemagglutinin protein, wherein the unmodified hemagglutinin protein is obtained from at least one strain of influenza virus, wherein the polypeptide of the modified hemagglutinin protein has at least 80%, or at least 90%, or at least 95%, or at least 97% sequence identity to at least one sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30.

2. (canceled)

3. The polypeptide fragment as claimed in claim 1, wherein the linker peptide is 2 to 9 amino acids in length.

4. The polypeptide fragment as claimed in claim 1, wherein the at least one strain of influenza virus comprises A/Hawaii/70/2019, A/Hong Kong/45/2019, B/Washington/02/2019, B/Phuket/3073/2013, A/Wisconsin/588/2019, A/Cambodia/e0826360/2020, A/Darwin/6/2021, B/Austria/1359417/2021, A/California/7/2009, A/Hong Kong/4801/2014, B/Brisbane/60/2008, B/Phuket/3073/2013, A/Michigan/45/2015, A/Singapore/INFINM-16-0019/2016, A/Switzerland/8060/2017, B/Colorado/06/2017, B/Phuket/3073/2013, A/Brisbane/02/2018, A/South Australia/34/2019, A/Kansas/14/2017, or any future or past seasonal influenza vaccine strains recommended by WHO.

5. The polypeptide fragment as claimed in claim 1, wherein the polypeptide further comprises a HRV3C recognition sequence having an amino acid sequence as set forth in SEQ ID NO: 35, wherein the polypeptide is derived by cleaving a His tag, and wherein HRV3C recognition sequence is present downstream of a linker.

6. The polypeptide fragment as claimed in claim 1, wherein the polypeptide fragment is in a form of a trimer or higher order aggregates.

7. A nucleic acid fragment encoding the polypeptide fragment as claimed in claim 1, wherein the nucleic acid fragment comprises:
(a) a nucleic acid sequence as set forth in SEQ ID NO: 1 encoding the polypeptide fragment having an amino acid sequence as set forth in SEQ ID NO: 2;
(b) a nucleic acid sequence as set forth in SEQ ID NO: 3 encoding the polypeptide fragment having an amino acid sequence as set forth in SEQ ID NO: 4;
(c) a nucleic acid sequence as set forth in SEQ ID NO: 5 encoding the polypeptide fragment having an amino acid sequence as set forth in SEQ ID NO: 6;
(d) a nucleic acid sequence as set forth in SEQ ID NO: 7 encoding the polypeptide fragment having an amino acid sequence as set forth in SEQ ID NO: 8;
(e) a nucleic acid sequence as set forth in SEQ ID NO: 21 encoding the polypeptide fragment having an amino acid sequence as set forth in SEQ ID NO: 22;
(f) a nucleic acid sequence as set forth in SEQ ID NO: 23 encoding the polypeptide fragment having an amino acid sequence as set forth in SEQ ID NO: 24;
(g) a nucleic acid sequence as set forth in SEQ ID NO: 27 encoding the polypeptide fragment having an amino acid sequence as set forth in SEQ ID NO: 28;
(h) a nucleic acid sequence as set forth in SEQ ID NO: 9 encoding the polypeptide fragment having an amino acid sequence as set forth in SEQ ID NO: 10;
(i) a nucleic acid sequence as set forth in SEQ ID NO: 11 encoding the polypeptide fragment having an amino acid sequence as set forth in SEQ ID NO: 12;
(i) a nucleic acid sequence as set forth in SEQ ID NO: 13 encoding the polypeptide fragment having an amino acid sequence as set forth in SEQ ID NO: 14;
(k) a nucleic acid sequence as set forth in SEQ ID NO: 15 encoding the polypeptide fragment having an amino acid sequence as set forth in SEQ ID NO: 16;
(l) a nucleic acid sequence as set forth in SEQ ID NO: 17 encoding the polypeptide fragment having an amino acid sequence as set forth in SEQ ID NO: 18;
(m) a nucleic acid sequence as set forth in SEQ ID NO: 19 encoding the polypeptide fragment having an amino acid sequence as set forth in SEQ ID NO: 20;
(n) a nucleic acid sequence as set forth in SEQ ID NO: 25 encoding the polypeptide fragment having an amino acid sequence as set forth in SEQ ID NO: 26: or
(o) a nucleic acid sequence as set forth in SEQ ID NO: 29 encoding the polypeptide fragment having an amino acid sequence as set forth in SEQ ID NO: 30.

8-9. (canceled)

10. A recombinant construct comprising a nucleic acid fragment encoding a polypeptide fragment as claimed in claim 1, operably linked to a promoter and a nucleic acid sequence encoding a signal peptide.

11. The recombinant construct as claimed in claim 10, wherein the nucleic acid fragment comprises at least one nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, and SEQ ID NO: 29, and/or wherein the signal peptide is encoded by a nucleic acid sequence as set forth in SEQ ID NO: 32.

12. (canceled)

13. A recombinant vector comprising the recombinant construct as claimed in a claim 10.

14. A recombinant host cell comprising the recombinant vector as claimed in claim 13, wherein the recombinant host cell is selected from the group consisting of insect cell, bacterial cell, yeast cell, and mammalian cell, wherein the insect cell is selected from the group consisting of Expi-Sf9®, Sf9, High Five®, Sf21, and S2, wherein the bacterial cell is Escherichia coli, wherein the yeast cell is selected from the group consisting of Pichia X33, Pichia GlycoSwitch®, Pichia SuperMan5®, DSMZ 70382, GS115, KM71, KM71H, BG09, GS190, GS200, JC220, JC254, JC227, JC300-JC308, YJN165, and CBS7435, and wherein the mammalian cell is selected from the group consisting of Expi293F® Expi-CHO-S®, CHO-K1, CHO-S, HEK293F®, CHO^{BC}™, SLIM™, SPOT™, SP2/0, Sp2/0-Ag14, CHO DG44, HEK 293S, HEK 293 Gnt1^{-/-}, HEK293-EBNA1, CHOL-NSO, and NSO.

15-16. (canceled)

17. An immunogenic composition comprising a plurality of polypeptide fragments as claimed in claim 1, and a pharmaceutically acceptable carrier, wherein the pharmaceutically acceptable carrier comprises an adjuvant, an excipient, or a combination thereof.

18-20. (canceled)

21. The immunogenic composition as claimed in claim 17, wherein the adjuvant is selected from the group consisting of squalene-in-water emulsion (SWE) adjuvant and chemically equivalent adjuvant.

22. (canceled)

23. The immunogenic composition as claimed in claim 17, wherein immunogenic composition is in form of a vaccine, wherein the vaccine is a multivalent vaccine or a quadrivalent vaccine.

24. (canceled)

25. A method for producing the immunogenic composition as claimed in claim 17, wherein the method comprises: (a) culturing a recombinant host cell, wherein the recombinant host cell is selected from the group consisting of insect cell, bacterial cell, yeast cell, and mammalian cell, wherein the insect cell is selected from the group consisting of Expi-Sf9®, Sf9, High Five®, Sf21, and S2, wherein the bacterial cell is Escherichia coli, wherein the yeast cell is selected from the group consisting of Pichia X33, Pichia GlycoSwitch®, Pichia SuperMan5®, DSMZ 70382, GS115, KM71, KM71H, BG09, GS190, GS200, JC220, JC254, JC227, JC300-JC308, YJN165, and CBS7435, and wherein the mammalian cell is selected from the group consisting of Expi293F® Expi-CHO-S®, CHO-K1, CHO-S, HEK293F®, CHO^{BC}™, SLIM™, SPOT™, SP2/0, Sp2/0-Ag14, CHO DG44, HEK 293S, HEK 293 Gnt1^{-/-}, HEK293-EBNA1, CHOL-NSO, and NSO, wherein the recombinant host cell comprises the recombinant construct or the recombinant vector comprising a nucleic acid fragment encoding a polypeptide fragment comprising an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30, under suitable conditions to obtain a polypeptide fragment comprising a polypeptide of a modified hemagglutinin protein, wherein the modified hemagglutinin protein comprises a linker peptide replacing a polypeptide in a cytoplasmic domain of an unmodified hemagglutinin protein, wherein the unmodified hemagglutinin protein is obtained from at least one strain of influenza virus, wherein the polypeptide of the modified hemagglutinin protein has at least 80%, or at least 90%, or at least 95%, or at least 97% sequence identity to at least one sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, and SEQ ID NO: 30; (b) subjecting the polypeptide to purification; and (c) contacting the polypeptide of step (b) with a pharmaceutically acceptable carrier for obtaining the immunogenic composition.

26. (canceled)

27. A method for eliciting an immune response to protect against influenza disease in a subject, the method comprising administering the subject with a pharmaceutically effective amount of the immunogenic composition as claimed in claim 17.

28. The method as claimed in claim 27, wherein the immunogenic composition is administered by a mode selected from the group consisting of intranasal, parenteral, subcutaneous, intramuscular, and intradermal.

29. A kit comprising the polypeptide as claimed in claim 1.

30-31. (canceled)

32. The recombinant construct as claimed in claim 11, wherein the recombinant construct further comprises an HRV3C recognition sequence.

33. The recombinant construct as claimed in claim 11, wherein the recombinant construct further comprises a histidine tag sequence.

34. A kit comprising the immunogenic composition as claimed in claim 17, and an instruction leaflet.
